SEQUENCE LISTING (173 sequences)

SEQ ID NO: 1 - protein, 237 aa, Artificial Sequence
  Control/VB1026
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 2 - protein, 19 aa, Artificial Sequence
  Signal peptide
MNFGLRLIFLVLTLKGVQC

SEQ ID NO: 3 - protein, 22 aa, Artificial Sequence
  Signal peptide
MDAMKRGLCCVLLLCGAVFVSP

SEQ ID NO: 4 - protein, 26 aa, Homo sapiens
MTVLAPAWSPTTYLLLLLLLSSGLSG

SEQ ID NO: 5 - protein, 528 aa, Artificial Sequence
  VB4194
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKSWIHCWKYLSVQSQLFRGSSLLFRRVGGGGSGGGGSNNLQKYIEIYVQKINPSRLPVVIGGLLGGGGSGGGGSEVIQTSKYYMRDVIAIESAWLLELAPHGGGGSGGGGSVILPQAPSGPSYATYLQPAQAQMLTPPGGGGSGGGGSFVSPMAHYVPGIMAIESVVARFQFIVPGGGGSGGGGSGDVKIHAHKVVLANISPYFKAMFTGNLGGGGSGGGGSTPLRKHTVHAIRKFYLEFKGSSPPPRLGGGGSGGGGSKIYEFDYHLYGQNITMIMTSVSGHLLA

SEQ ID NO: 6 - protein, 733 aa, Artificial Sequence
  VB4168
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKSWIHCWKYLSVQSQLFRGSSLLFRRVGGGGSGGGGSNNLQKYIEIYVQKINPSRLPVVIGGLLGGGGSGGGGSEVIQTSKYYMRDVIAIESAWLLELAPHGGGGSGGGGSVILPQAPSGPSYATYLQPAQAQMLTPPGGGGSGGGGSFVSPMAHYVPGIMAIESVVARFQFIVPGGGGSGGGGSGDVKIHAHKVVLANISPYFKAMFTGNLGGGGSGGGGSTPLRKHTVHAIRKFYLEFKGSSPPPRLGGGGSGGGGSKIYEFDYHLYGQNITMIMTSVSGHLLAGSGEGRGSLLTCGDVEENPGPMTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 7 - protein, 896 aa, Artificial Sequence
  VB4169
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKSWIHCWKYLSVQSQLFRGSSLLFRRVGGGGSGGGGSNNLQKYIEIYVQKINPSRLPVVIGGLLGGGGSGGGGSEVIQTSKYYMRDVIAIESAWLLELAPHGGGGSGGGGSVILPQAPSGPSYATYLQPAQAQMLTPPGGGGSGGGGSFVSPMAHYVPGIMAIESVVARFQFIVPGGGGSGGGGSGDVKIHAHKVVLANISPYFKAMFTGNLGGGGSGGGGSTPLRKHTVHAIRKFYLEFKGSSPPPRLGGGGSGGGGSKIYEFDYHLYGQNITMIMTSVSGHLLAGSGEGRGSLLTCGDVEENPGPMTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPGSGATNFSLLKQAGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 8 - protein, 1010 aa, Artificial Sequence
  VB4170
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKSWIHCWKYLSVQSQLFRGSSLLFRRVGGGGSGGGGSNNLQKYIEIYVQKINPSRLPVVIGGLLGGGGSGGGGSEVIQTSKYYMRDVIAIESAWLLELAPHGGGGSGGGGSVILPQAPSGPSYATYLQPAQAQMLTPPGGGGSGGGGSFVSPMAHYVPGIMAIESVVARFQFIVPGGGGSGGGGSGDVKIHAHKVVLANISPYFKAMFTGNLGGGGSGGGGSTPLRKHTVHAIRKFYLEFKGSSPPPRLGGGGSGGGGSKIYEFDYHLYGQNITMIMTSVSGHLLAGSGEGRGSLLTCGDVEENPGPMTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPGSGATNFSLLKQAGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGSGQCTNYALLKLAGDVESNPGPMKISAAALTIILTAAALCTPAPASPYGSDTTPCCFAYLSLALPRAHVKEYFYTSSKCSNLAVVFVTRRNRQVCANPEKKWVQEYINYLEMS

SEQ ID NO: 9 - protein, 18 aa, Artificial Sequence
  Self-cleaving peptide
EGRGSLLTCGDVEENPGP

SEQ ID NO: 10 - protein, 158 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 11 - protein, 19 aa, Artificial Sequence
  Self-cleaving peptide
ATNFSLLKQAGDVEENPGP

SEQ ID NO: 12 - protein, 17 aa, Mus Musculus
MWLQNLLFLGIVVYSLS

SEQ ID NO: 13 - protein, 124 aa, Mus Musculus
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 14 - protein, 20 aa, Artificial Sequence
  Self-cleaving peptide
QCTNYALLKLAGDVESNPGP

SEQ ID NO: 15 - protein, 23 aa, Mus Musculus
MKISAAALTIILTAAALCTPAPA

SEQ ID NO: 16 - protein, 68 aa, Mus Musculus
SPYGSDTTPCCFAYLSLALPRAHVKEYFYTSSKCSNLAVVFVTRRNRQVCANPEKKWVQEYINYLEMS

SEQ ID NO: 17 - protein, 10 aa, Artificial Sequence
  Linker
GGGGSGGGGS

SEQ ID NO: 18 - protein, 690 aa, Artificial Sequence
  VB4202
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKSWIHCWKYLSVQSQLFRGSSLLFRRVGGGGSGGGGSNNLQKYIEIYVQKINPSRLPVVIGGLLGGGGSGGGGSEVIQTSKYYMRDVIAIESAWLLELAPHGGGGSGGGGSVILPQAPSGPSYATYLQPAQAQMLTPPGGGGSGGGGSFVSPMAHYVPGIMAIESVVARFQFIVPGGGGSGGGGSGDVKIHAHKVVLANISPYFKAMFTGNLGGGGSGGGGSTPLRKHTVHAIRKFYLEFKGSSPPPRLGGGGSGGGGSKIYEFDYHLYGQNITMIMTSVSGHLLAGSGEGRGSLLTCGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 19 - protein, 501 aa, Artificial Sequence [sequence omitted]

SEQ ID NO: 20 - protein, 706 aa, Artificial Sequence
  VB4195
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQLGSGEGRGSLLTCGDVEENPGPMTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 21 - protein, 869 aa, Artificial Sequence
  VB4196
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQLGSGEGRGSLLTCGDVEENPGPMTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPGSGATNFSLLKQAGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 22 - protein, 663 aa, Artificial Sequence
  VB4204
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQLGSGEGRGSLLTCGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 23 - protein, 613 aa, Artificial Sequence
  VB4205
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQLGSGEGRGSLLTCGDVEENPGPMKISAAALTIILTAAALCTPAPASPYGSDTTPCCFAYLSLALPRAHVKEYFYTSSKCSNLAVVFVTRRNRQVCANPEKKWVQEYINYLEMS

SEQ ID NO: 24 - protein, 404 aa, Artificial Sequence
  VB4208
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLGSGEGRGSLLTCGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 25 - DNA, 210 bp, Artificial Sequence
  Encoding part of SEQ ID NO 1
gcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgcc

SEQ ID NO: 26 - DNA, 81 bp, Artificial Sequence
  Encoding part of SEQ ID NO 1
gagctcaaaaccccacttggtgacacaactcacacagagcccaaatcttgtgacacacctcccccgtgcccaaggtgccca

SEQ ID NO: 27 - DNA, 321 bp, Artificial Sequence
  Encoding part of SEQ ID NO 1
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 28 - DNA, 432 bp, Artificial Sequence
  Encoding part of SEQ ID NO 1
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 29 - DNA, 69 bp, Artificial Sequence
  Encoding part of SEQ ID NO 1
atgcaggtctccactgctgcccttgccgtcctcctctgcaccatggctctctgcaaccaggtcctctct

SEQ ID NO: 30 - protein, 223 aa, Artificial Sequence
  SARS-COV-2 RBD (amino acids 319-542)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 31 - protein, 627 aa, Artificial Sequence
  TECH001-CV021
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGEGRGSLLTCGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 32 - protein, 1054 aa, Artificial Sequence
  TECH001-CV022
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGEGRGSLLTCGDVEENPGPMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAEGRGSLLTCGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 33 - protein, 632 aa, Artificial Sequence
  TECH001-CV023
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGEGRGSLLTCGDVEENPGPMERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 34 - protein, 22 aa, Mus Musculus
MCQSRYLLFLATLALLNHLSLA

SEQ ID NO: 35 - protein, 193 aa, Mus Musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 36 - protein, 22 aa, Mus Musculus
MCPQKLTISWFAIVLLVSPLMA

SEQ ID NO: 37 - protein, 313 aa, Mus Musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 38 - protein, 17 aa, Mus Musculus
MERTLVCLVVIFLGTVA

SEQ ID NO: 39 - protein, 129 aa, Mus Musculus
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 40 - protein, 17 aa, Homo sapiens
MWLQSLLLLGTVACSIS

SEQ ID NO: 41 - protein, 127 aa, Homo sapiens [sequence omitted]

SEQ ID NO: 42 - protein, 23 aa, Homo sapiens
MKVSAAALAVILIATALCAPASA

SEQ ID NO: 43 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 44 - protein, 22 aa, Homo sapiens
MCPARSLLLVATLVLLDHLSLA

SEQ ID NO: 45 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 46 - protein, 22 aa, Homo sapiens
MCHQQLVISWFSLVFLASPLVA

SEQ ID NO: 47 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 48 - protein, 24 aa, Homo sapiens
MRSSPGNMERIVICLMVIFLGTLV

SEQ ID NO: 49 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 50 - protein, 8 aa, Artificial Sequence
  self-cleaving peptide
  misc_feature (2)..(2): Xaa can be any naturally occurring amino acid
  misc_feature (4)..(4): Xaa can be any naturally occurring amino acid
DXEXNPGP

SEQ ID NO: 51 - protein, 22 aa, Artificial Sequence
  self-cleaving peptide
VKQTLNFDLLKLAGDVESNPGP

SEQ ID NO: 52 - protein, 21 aa, Artificial Sequence
  self-cleaving peptide
GSGEGRGSLLTCGDVEENPGP

SEQ ID NO: 53 - protein, 22 aa, Artificial Sequence
  self-cleaving peptide
GSGATNFSLLKQAGDVEENPGP

SEQ ID NO: 54 - protein, 23 aa, Artificial Sequence
  self-cleaving peptide
GSGQCTNYALLKLAGDVESNPGP

SEQ ID NO: 55 - protein, 25 aa, Artificial Sequence
  self-cleaving peptide
GSGVKQTLNFDLLKLAGDVESNPGP

SEQ ID NO: 56 - protein, 27 aa, Artificial Sequence
  trimerization unit
GYIPEAPRDGQAYVRKDGEWVLLSTFL

SEQ ID NO: 57 - DNA, 117 bp, Artificial Sequence
  tetramerization unit
aagcctctggacggagagtatttcactctccagatccggggccccgaaaggttcgaaatgttccgggagcttaacgaggccttggagctgaaagacgcacaggccggaaaggaaccg

SEQ ID NO: 58 - protein, 5 aa, Artificial Sequence
  Linker
GGGGS

SEQ ID NO: 59 - protein, 5 aa, Artificial Sequence
  Linker
GGGSS

SEQ ID NO: 60 - protein, 5 aa, Artificial Sequence
  Linker
GGGSG

SEQ ID NO: 61 - protein, 5 aa, Artificial Sequence
  Linker
GGSGG

SEQ ID NO: 62 - protein, 6 aa, Artificial Sequence
  Linker
SGSSGS

SEQ ID NO: 63 - protein, 6 aa, Artificial Sequence
  Linker
  SITE (1)..(4): can be repeated m times, where m is an integer from 1 to 5
EAAKGS

SEQ ID NO: 64 - protein, 5 aa, Artificial Sequence
  Linker
  SITE (1)..(5): can be repeated m times, where m is an integer from 1 to 5
GGGGS

SEQ ID NO: 65 - protein, 5 aa, Artificial Sequence
  Linker
  SITE (1)..(5): can be repeated m times, where m is an integer from 1 to 5
GGGSS

SEQ ID NO: 66 - protein, 5 aa, Artificial Sequence
  Linker
  SITE (1)..(5): can be repeated m times, where m is an integer from 1 to 5
GGSGG

SEQ ID NO: 67 - protein, 5 aa, Artificial Sequence
  Linker
  SITE (1)..(5): can be repeated m times, where m is an integer from 1 to 5
GGGSG

SEQ ID NO: 68 - protein, 6 aa, Artificial Sequence
  Linker
SGSSGS

SEQ ID NO: 69 - protein, 5 aa, Artificial Sequence
  Linker
LGGGS

SEQ ID NO: 70 - protein, 5 aa, Artificial Sequence
  Linker
GLGGS

SEQ ID NO: 71 - protein, 5 aa, Artificial Sequence
  Linker
GGLGS

SEQ ID NO: 72 - protein, 5 aa, Artificial Sequence
  Linker
GGGLS

SEQ ID NO: 73 - protein, 5 aa, Artificial Sequence
  Linker
GGGGL

SEQ ID NO: 74 - protein, 5 aa, Artificial Sequence
  Linker
LGGSG

SEQ ID NO: 75 - protein, 5 aa, Artificial Sequence
  Linker
GLGSG

SEQ ID NO: 76 - protein, 5 aa, Artificial Sequence
  Linker
GGLSG

SEQ ID NO: 77 - protein, 5 aa, Artificial Sequence
  Linker
GGGLG

SEQ ID NO: 78 - protein, 5 aa, Artificial Sequence
  Linker
GGGSL

SEQ ID NO: 79 - protein, 5 aa, Artificial Sequence
  Linker
LGGSS

SEQ ID NO: 80 - protein, 5 aa, Artificial Sequence
  Linker
GLGSS

SEQ ID NO: 81 - protein, 5 aa, Artificial Sequence
  Linker
GGLSS

SEQ ID NO: 82 - protein, 9 aa, Artificial Sequence
  T cell epitope
CTELKLSDY

SEQ ID NO: 83 - protein, 9 aa, Artificial Sequence
  T cell epitope
NLVPMVATV

SEQ ID NO: 84 - protein, 9 aa, Artificial Sequence
  T cell epitope
KLVANNTRL

SEQ ID NO: 85 - protein, 5 aa, Artificial Sequence
  Linker
LGLGS

SEQ ID NO: 86 - protein, 5 aa, Artificial Sequence
  Linker
GLGLS

SEQ ID NO: 87 - protein, 5 aa, Artificial Sequence
  Linker
GLLGS

SEQ ID NO: 88 - protein, 5 aa, Artificial Sequence
  Linker
LGGLS

SEQ ID NO: 89 - protein, 5 aa, Artificial Sequence
  Linker
GLGGL

SEQ ID NO: 90 - protein, 5 aa, Artificial Sequence
  Linker
LGLSG

SEQ ID NO: 91 - protein, 5 aa, Artificial Sequence
  Linker
GLLSG

SEQ ID NO: 92 - protein, 5 aa, Artificial Sequence
  Linker
GGLSL

SEQ ID NO: 93 - protein, 5 aa, Artificial Sequence
  Linker
GGLLG

SEQ ID NO: 94 - protein, 5 aa, Artificial Sequence
  Linker
GLGSL

SEQ ID NO: 95 - protein, 5 aa, Artificial Sequence
  Linker
LGLSS

SEQ ID NO: 96 - protein, 5 aa, Artificial Sequence
  Linker
GGLLS

SEQ ID NO: 97 - protein, 10 aa, Artificial Sequence
  Linker
LGGGSGGGGS

SEQ ID NO: 98 - protein, 10 aa, Artificial Sequence
  Linker
GLGGSGGGGS

SEQ ID NO: 99 - protein, 10 aa, Artificial Sequence
  Linker
GGLGSGGGGS

SEQ ID NO: 100 - protein, 10 aa, Artificial Sequence
  Linker
GGGLSGGGGS

SEQ ID NO: 101 - protein, 10 aa, Artificial Sequence
  Linker
GGGGLGGGGS

SEQ ID NO: 102 - protein, 10 aa, Artificial Sequence
  Linker
LGGSGGGGSG

SEQ ID NO: 103 - protein, 10 aa, Artificial Sequence
  Linker
GLGSGGGGSG

SEQ ID NO: 104 - protein, 10 aa, Artificial Sequence
  Linker
GGLSGGGGSG

SEQ ID NO: 105 - protein, 10 aa, Artificial Sequence
  Linker
GGGLGGGGSG

SEQ ID NO: 106 - protein, 10 aa, Artificial Sequence
  Linker
GGGSLGGGSG

SEQ ID NO: 107 - protein, 10 aa, Artificial Sequence
  Linker
LGGSSGGGSS

SEQ ID NO: 108 - protein, 10 aa, Artificial Sequence
  Linker
GLGSSGGGSS

SEQ ID NO: 109 - protein, 10 aa, Artificial Sequence
  Linker
GGLSSGGGSS

SEQ ID NO: 110 - protein, 10 aa, Artificial Sequence
  Linker
GGGLSGGGSS

SEQ ID NO: 111 - protein, 10 aa, Artificial Sequence
  Linker
GGGSLGGGSS

SEQ ID NO: 112 - protein, 10 aa, Artificial Sequence
  Linker
LGGGSLGGGS

SEQ ID NO: 113 - protein, 10 aa, Artificial Sequence
  Linker
GLGGSGLGGS

SEQ ID NO: 114 - protein, 10 aa, Artificial Sequence
  Linker
GGLGSGGLGS

SEQ ID NO: 115 - protein, 10 aa, Artificial Sequence
  Linker
GGGLSGGGLS

SEQ ID NO: 116 - protein, 10 aa, Artificial Sequence
  Linker
GGGGLGGGGL

SEQ ID NO: 117 - protein, 10 aa, Artificial Sequence
  Linker
LGGSGLGGSG

SEQ ID NO: 118 - protein, 10 aa, Artificial Sequence
  Linker
GLGSGGLGSG

SEQ ID NO: 119 - protein, 10 aa, Artificial Sequence
  Linker
GGLSGGGLSG

SEQ ID NO: 120 - protein, 10 aa, Artificial Sequence
  Linker
GGGLGGGGLG

SEQ ID NO: 121 - protein, 10 aa, Artificial Sequence
  Linker
GGGSLGGGSL

SEQ ID NO: 122 - protein, 10 aa, Artificial Sequence
  Linker
LGGSSLGGSS

SEQ ID NO: 123 - protein, 10 aa, Artificial Sequence
  Linker
GLGSSGLGSS

SEQ ID NO: 124 - protein, 10 aa, Artificial Sequence
  Linker
GGLSSGGLSS

SEQ ID NO: 125 - protein, 6 aa, Artificial Sequence
  Linker
GSGGGA

SEQ ID NO: 126 - protein, 12 aa, Artificial Sequence
  Linker
GSGGGAGSGGGA

SEQ ID NO: 127 - protein, 18 aa, Artificial Sequence
  Linker
GSGGGAGSGGGAGSGGGA

SEQ ID NO: 128 - protein, 24 aa, Artificial Sequence
  Linker
GSGGGAGSGGGAGSGGGAGSGGGA

SEQ ID NO: 129 - protein, 10 aa, Artificial Sequence
  Linker
GENLYFQSGG

SEQ ID NO: 130 - protein, 10 aa, Artificial Sequence
  Linker
SGGGSSGGGS

SEQ ID NO: 131 - protein, 10 aa, Artificial Sequence
  Linker
SSGGGSSGGG

SEQ ID NO: 132 - protein, 10 aa, Artificial Sequence
  Linker
GGSGGGGSGG

SEQ ID NO: 133 - protein, 10 aa, Artificial Sequence
  Linker
GSGSGSGSGS

SEQ ID NO: 134 - protein, 10 aa, Artificial Sequence
  Linker
GGGSSGGGSG

SEQ ID NO: 135 - protein, 6 aa, Artificial Sequence
  Linker
GGGSSS

SEQ ID NO: 136 - protein, 15 aa, Artificial Sequence
  Linker
GGGSSGGGSSGGGSS

SEQ ID NO: 137 - protein, 8 aa, Artificial Sequence
  Linker
GLGGLAAA

SEQ ID NO: 138 - protein, 12 aa, Artificial Sequence
  Linker
KPEPKPAPAPKP

SEQ ID NO: 139 - protein, 12 aa, Artificial Sequence
  Linker
AEAAAKEAAAKA

SEQ ID NO: 140 - protein, 5 aa, Artificial Sequence
  Linker
  SITE (1)..(5): can be repeated m times, where m is an integer from 1 to 5
EAAAK

SEQ ID NO: 141 - protein, 15 aa, Artificial Sequence
  Linker
PSRLEEELRRRLTEP

SEQ ID NO: 142 - protein, 8 aa, Artificial Sequence
  Linker
SACYCELS

SEQ ID NO: 143 - protein, 16 aa, Artificial Sequence
  Linker
TQKSLSLSPGKGLGGL

SEQ ID NO: 144 - protein, 13 aa, Artificial Sequence
  Linker
SLSLSPGKGLGGL

SEQ ID NO: 145 - protein, 36 aa, Artificial Sequence
  Linker
GGSAGGSGSGSSGGSSGASGTGTAGGTGSGSGTGSG

SEQ ID NO: 146 - protein, 36 aa, Artificial Sequence
  Linker
GGSGGGSEGGGSEGGGSEGGGSEGGGSEGGGSGGGS

SEQ ID NO: 147 - protein, 12 aa, Artificial Sequence
  Linker
ELKTPLGDTTHT

SEQ ID NO: 148 - protein, 15 aa, Artificial Sequence
  Linker
EPKSCDTPPPCPRCP

SEQ ID NO: 149 - protein, 5 aa, Artificial Sequence
  Linker
GLSGL

SEQ ID NO: 150 - protein, 5 aa, Artificial Sequence
  Linker
EAAAK

SEQ ID NO: 151 - protein, 7 aa, Artificial Sequence
  Linker
  SITE (1)..(5): can be repeated m times, where m is an integer from 1 to 5
EAAAKGS

SEQ ID NO: 152 - protein, 17 aa, Artificial Sequence
  Linker
GPSRLEEELRRRLTEPG

SEQ ID NO: 153 - protein, 12 aa, Artificial Sequence
  Linker
HEYGAEALERAG

SEQ ID NO: 154 - protein, 27 aa, Artificial Sequence
  CT26 epitope
VILPQAPSGPSYATYLQPAQAQMLTPP

SEQ ID NO: 155 - protein, 27 aa, Artificial Sequence
  CT26 epitope
EVIQTSKYYMRDVIAIESAWLLELAPH

SEQ ID NO: 156 - protein, 27 aa, Artificial Sequence
  CT26 epitope
KSWIHCWKYLSVQSQLFRGSSLLFRRV

SEQ ID NO: 157 - protein, 27 aa, Artificial Sequence
  CT26 epitope
GDVKIHAHKVVLANISPYFKAMFTGNL

SEQ ID NO: 158 - protein, 27 aa, Artificial Sequence
  CT26 epitope
FVSPMAHYVPGIMAIESVVARFQFIVP

SEQ ID NO: 159 - protein, 27 aa, Artificial Sequence
  CT26 epitope
KIYEFDYHLYGQNITMIMTSVSGHLLA

SEQ ID NO: 160 - protein, 27 aa, Artificial Sequence
  CT26 epitope
NNLQKYIEIYVQKINPSRLPVVIGGLL

SEQ ID NO: 161 - protein, 27 aa, Artificial Sequence
  CT26 epitope
TPLRKHTVHAIRKFYLEFKGSSPPPRL

SEQ ID NO: 162 - protein, 9 aa, Artificial Sequence
  HPV epitope
RAHYNIVTF

SEQ ID NO: 163 - protein, 15 aa, Artificial Sequence
  HPV epitope
MFQDPQERPRKLPQL

SEQ ID NO: 164 - protein, 15 aa, Artificial Sequence
  HPV epitope
RPRKLPQLCTELQTT

SEQ ID NO: 165 - protein, 15 aa, Artificial Sequence
  HPV epitope
LCTELQTTIHDIILE

SEQ ID NO: 166 - protein, 15 aa, Artificial Sequence
  HPV epitope
TIHDIILECVYCKQQ

SEQ ID NO: 167 - protein, 15 aa, Artificial Sequence
  HPV epitope
ECVYCKQQLLRREVY

SEQ ID NO: 168 - protein, 15 aa, Artificial Sequence
  HPV epitope
QLLRREVYDFARRDL

SEQ ID NO: 169 - protein, 15 aa, Artificial Sequence
  HPV epitope
YDFARRDLCIVYRDG

SEQ ID NO: 170 - protein, 15 aa, Artificial Sequence
  HPV epitope
LCIVYRDGNPYAVRD

SEQ ID NO: 171 - protein, 15 aa, Artificial Sequence
  HPV epitope
GNPYAVRDKCLKFYS

SEQ ID NO: 172 - protein, 15 aa, Artificial Sequence
  HPV epitope
DKCLKFYSKISEYRH

SEQ ID NO: 173 - protein, 8 aa, Artificial Sequence
  self-cleaving peptide
DVESNPGP

(19) United States
(12) Patent Application Publication
(10) Pub. No.: US 2025/0222085 A1
(43) Pub. Date: Jul. 10, 2025

(54) CO-EXPRESSION OF CONSTRUCTS AND IMMUNOSTIMULATORY COMPOUNDS

(71) Applicant: Nykode Therapeutics ASA, Oslo (NO)

(72) Inventors: Agnete Brunsvik Fredriksen, Rælingen (NO); Audun Trygge Haugen Bersaas, Oslo (NO); Stine Granum, Oslo (NO); Pierre Dillard, Oslo (NO)

(73) Assignee: Nykode Therapeutics ASA, Oslo (NO)

(21) Appl. No.: 18/560,279

(22) PCT Filed: May 10, 2022
(86) PCT No.: PCT/EP2022/062665

§ 371 Date: Nov. 10, 2023

(30) Foreign Application Priority Data
May 10, 2021 (DK) PA 2021 70221
Jul. 8, 2021 (DK) PA 2021 70364
Mar. 18, 2022 (DK) PA 2022 70113
Apr. 7, 2022 (DK) PA 2022 70186

(51) Int. Cl. A61K 39/00; A61K 39/12; A61P 35/00; C07K 14/47; C07K 14/52; C07K 14/535; C12N 7/00; C12N 15/86

(52) U.S. Cl. CPC A61K 39/001142; A61K 39/001139; A61K 39/12; A61P 35/00; C07K 14/4705; C07K 14/521; C07K 14/535; C12N 7/00; C12N 15/86; A61K 2039/53; A61K 2039/542; A61K 2039/543; A61K 2039/585; C12N 2710/20034; C12N 2840/203

(57) ABSTRACT

The present invention relates to vectors, such as DNA plasmids, comprising multiple nucleic acid sequences engineered to be co-expressed as separate molecules. Such separate molecules include a first polypeptide, wherein the first polypeptide comprises a targeting unit that targets antigen-presenting cells, a multimerization unit, such as dimerization unit, and an antigenic unit comprising one or more antigens or parts thereof, and one or more immunostimulatory compounds.

### TECHNICAL FIELD

The present invention relates to vectors, such as DNA plasmids, comprising multiple nucleic acid sequences of interest engineered to be co-expressed as separate molecules, to pharmaceutical compositions comprising such vectors and to the use of such vectors and such pharmaceutical compositions in the treatment or prevention of diseases.

### BACKGROUND

Both B cell (humoral/antibody mediated) and T cell responses are important components of protective responses against infections caused by pathogens. Specific antibodies against pathogen antigens can mediate a broad range of effector functions, such as e.g. a) direct neutralization of toxins or pathogens, b) neutralization of pathogen virulence factors, c) binding to and trapping of pathogens in mucins, d) activating complement to mediate anti-pathogen phagocytic clearance, degradation or lysis, e) activating neutrophil opsonophagocytosis, f) inducing macrophage opsonophagocytosis g) activating natural killer (NK) cell degranulation to kill infected cells, h) enhancing antigen update, processing and presentation by dendritic cells to T and B cells, i) inducing degranulation of mast cells, basophils and eosinophils in the setting of parasitic infections (L. Lu et al., Nat Rev Immunol 18(1), 2018, 46).

Complementing these activities, T cell responses are critical for limiting viral replication and infection by killing the infected cells, inducing apoptosis, releasing antiviral substances, and/or inducing increased intracellular lysis in already infected cells and thus help to prevent, reduce severity of or cure the disease. In addition, effective and long-lasting response in both arms of immunity usually requires additional support from T-helper (Th1 and Th2) lymphocytes.

Cytotoxic T lymphocytes (CTL) also play a significant role (F. Sheperd et al., Int J Mol Sci 21, 2020, 6144) with e.g. intracellular pathogens where MHC class I-restricted CD8+ T cells are critical for clearing bacterial infections and are known to provide protective immunity against a range of bacterial species. MHC class II restricted CD4+ T cells support memory CD8+ T cell responses and are important for protective immunity against bacterial infections. Naïve CD4+ T cells differentiate subsets of cells with effector capacity, such as T helper 1 (Th1) and Th2 cells. After binding specific T cell epitopes on the surface of antigen-presenting cells (APCs), Th1 and Th2 cells supply specific soluble cytokine signals that regulate the balance between antibody and CTL immunity. Thus, effective immunity involves multiple antigen recognition events of specific pathogen immunogenic determinants (epitopes) by T-helper cells followed by molecular recognition by B cells, CTL, or both.

Different types of lymphocytes (B cells, CTLs and Th cells) specifically recognize different types of epitopes of the pathogen. B cell epitopes can be categorized as linear or conformational epitopes, with linear epitopes often being parts of conformational B-cell epitopes in native proteins. Conformational epitopes are exposed structural features on the surface of pathogens such as a viral envelope, bacterial outer membrane or secreted bacterial toxins. T cell epitopes are short peptides from any protein of a pathogen, which only have to conform to the host antigen-processing and MHC binding mechanisms, most notably class I or class II MHC haplotype restriction mechanisms. Suitable T cell epitopes occur with an estimated frequency of about one per 200-500 amino acid sequence, depending on host population and pathogen.

Vaccines against pathogens comprise the pathogen or parts thereof, modified in such a manner that no harm or disease is caused, but ensuring that when the host is confronted with that infectious agent, the immune system can adequately neutralize it before it causes any ill effect. For more than a hundred years, vaccination has been performed by one of two approaches: either introducing specific antigens against which the immune system reacts directly; or introducing live attenuated infectious agents that replicate within the host without causing disease and synthesize the antigens that subsequently prime the immune system.

Recently, a radically new approach to vaccination has been developed. Polynucleotide sequences (DNA or RNA), encoding antigen(s) capable of stimulating an immune response, are directly introduced into appropriate tissues for in situ production of the target antigen. This approach offers a number of potential advantages over traditional approaches, including the stimulation of both B- and T-cell responses, improved vaccine stability, the absence of any infectious agent and the relative ease of large-scale manufacture.

Although treatment of cancer has been improved over the past few decades, particularly due to early detection and diagnosis, which has significantly increased the survival of cancer patients, only about 60% of such patients are alive 5 years after the diagnosis. Most of the cancer treatments in use are surgical procedures, radiation and cytotoxic chemotherapeutics, however they all have serious side effects. Recently, also treatments using antibodies directed towards known cancer associated antigens are used.

Within the last few years, cancer immunotherapies targeting cancer cells with the help of the patient's own immune system, i.e. cancer vaccines, have attracted interest. Such therapies may reduce or even eliminate some of the side-effects associated with traditional cancer treatments.

There is thus a need for efficient agents and medicaments which can be used both for treating or preventing an infectious disease, and for treating or preventing cancer.

The Vaccibody construct is a dimeric fusion protein consisting of two polypeptides, each comprising a targeting unit, which targets antigen-presenting cells, a dimerization unit and an antigenic unit, which comprises one or more antigens or parts thereof, and which is, after administration to a subject (e.g. an animal or human), efficient in generating an immune response against the antigens or parts thereof, e.g. epitopes, comprised in the antigenic unit. In another embodiment, the Vaccibody construct is a multimeric fusion protein consisting of multiple polypeptides, each comprising a targeting unit that targets antigen-presenting cells, a multimerization unit and an antigenic unit which comprises one or more antigens or parts thereof, and which, after administration to a subject, has shown to be efficient in generating an immune response against the antigens or parts thereof, e.g. epitopes, comprised in the antigenic unit.

The Vaccibody construct may be administered to a subject in the form of a polynucleotide encoding the polypeptide, e.g. a polynucleotide comprised in a vector, such as a DNA plasmid. After administration to host cells, e.g. administration to muscle cells of a subject, a polypeptide is expressed which, due to the multimerization unit, such as dimerization unit, forms a multimeric fusion protein, such as a dimer.

### SUMMARY

The present inventors have made the surprising observation that it is possible to enhance the overall immune response of a Vaccibody by co-expressing one or more immunostimulatory compounds from the same vector from which the Vaccibody is expressed.

In a first aspect, the present invention relates to a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit which comprises one or more antigens or parts thereof; and
(b) one or more further nucleic acid sequences encoding one or more immunostimulatory compounds,
   wherein the vector allows for the co-expression of the first polypeptide and the one or more immunostimulatory compounds as separate molecules.

In one embodiment, the vectors of the invention comprise a first nucleic acid sequence encoding a first polypeptide, wherein the first polypeptide comprises a targeting unit that targets antigen-presenting cells, a multimerization unit, such as a dimerization unit, and an antigenic unit comprising one or more disease-relevant antigens or parts thereof. Such vectors may be used, e.g. in the form of pharmaceutical compositions comprising such vector and a pharmaceutically acceptable carrier or diluent, for the prophylactic or therapeutic treatment of a disease, e.g. for the therapeutic treatment of cancer or for the prophylactic or therapeutic treatment of an infectious disease, by administering the composition to a subject in need of such prophylactic or therapeutic treatment.

### DESCRIPTION OF DRAWINGS

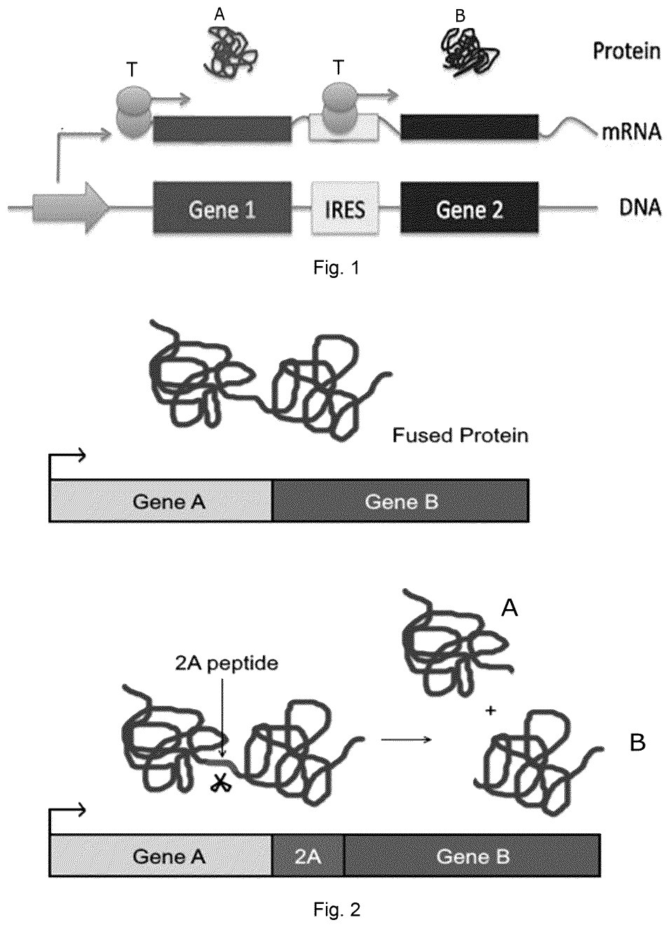

### FIG. 1 Co-Expression Elements for Use in the Vector of the Invention

Shows an IRES co-expression element for use in the vector of the invention, which is inserted in between two coding regions. When the mRNA has been produced, two ribosomes (T) are able to start translation at two separate sites on the mRNA and two proteins (A and B) are formed. A and B can for example be a first polypeptide and an immunostimulatory compound.

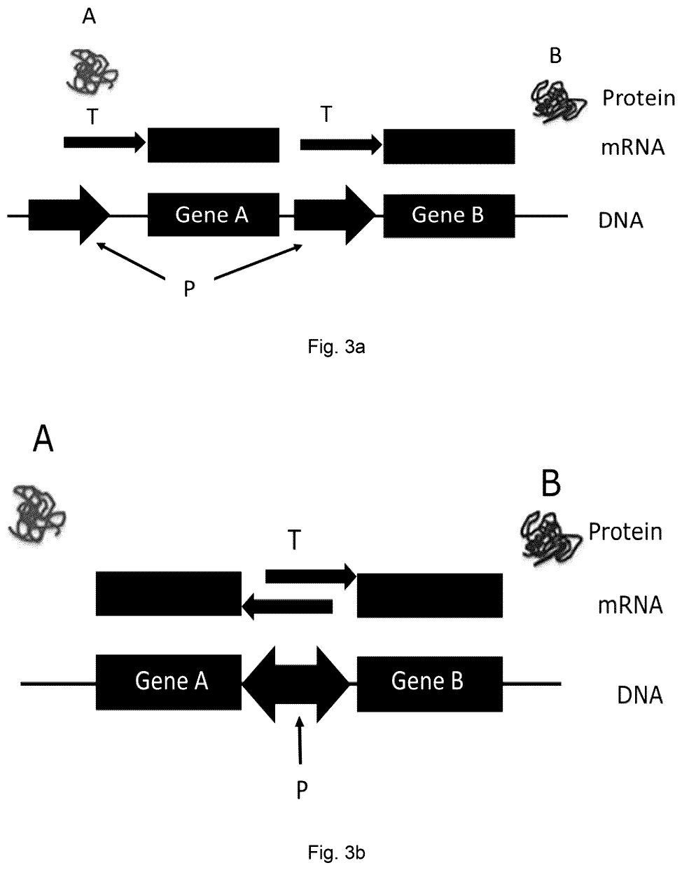

### FIG. 2 Co-Expression Element for Use in the Vector of the Invention

Shows a 2A self-cleaving peptide co-expression element for use in the vector of the invention, which is inserted between two genes. After transcription, one ribosome translates the mRNA and two proteins (A and B) are formed. Top of the figure shows how a fusion protein is formed if a 2A peptide sequence is not part of the coding sequence. A and B can for example be a first polypeptide and an immunostimulatory compound.

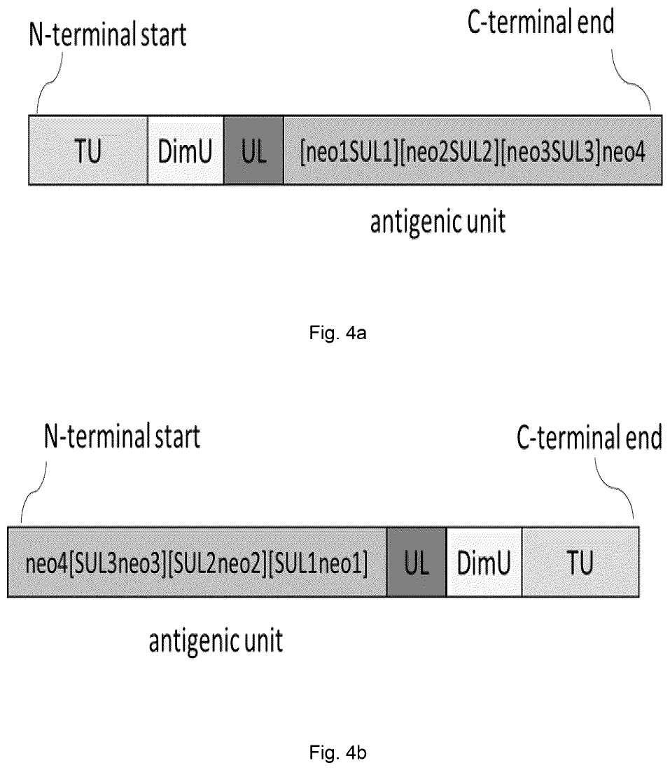

### FIGS. 3 Co-Expression Elements for Use in the Vector of the Invention

FIG. 3a shows a bidirectional promoter (P) co-expression element for use in the vector of the invention, which is located between two coding regions. One mRNA is produced and two ribosomes (T) are able to start translation in separate directions and two proteins (A and B) are formed. A and B can for example be a first polypeptide and an immunostimulatory compound. FIG. 3a shows two promoters (P), i.e. co-expression elements for use in the vector of the invention, which are located before two coding regions. Two mRNAs are produced and two ribosomes (T) are able to start translation at two different mRNAs and two proteins (A and B) are formed. A and B can for example be a first polypeptide and an immunostimulatory compound of the invention.

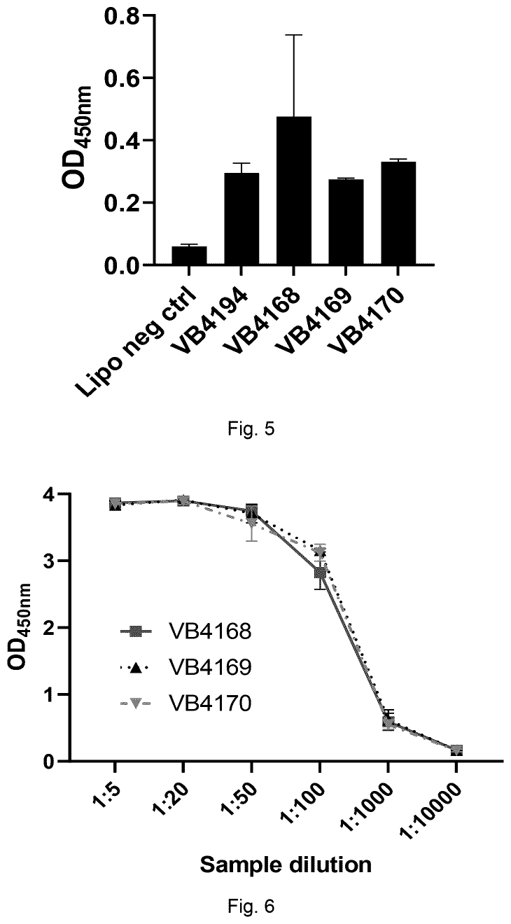

### FIG. 4 Embodiment of a First Polypeptide

Illustrates an embodiment of a first polypeptide encoded by the first nucleic acid sequence comprised in the vector of the invention.

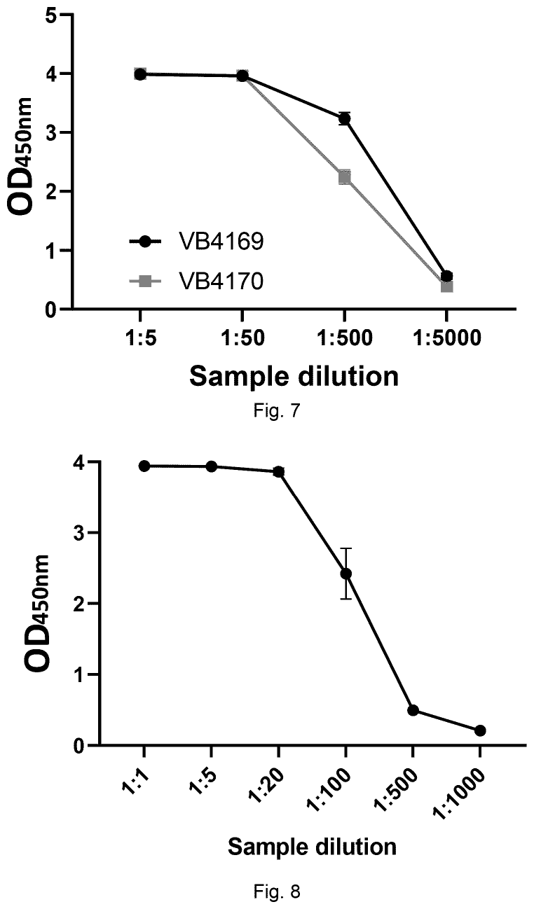

### FIG. 5 Expression and Secretion Levels of Proteins Encoded by a DNA Plasmid

Shows the protein expression and secretion levels of the first polypeptides encoded by DNA plasmids VB4194, VB4168, VB4169 and VB4170 detected in the supernatant of HEK293 cells transfected with said DNA plasmids by the enzyme-linked immunosorbent assay (ELISA) using mouse a-human IgG CH3 domain capture Ab (MCA878G) and a-human MIP-1α biotinylated capture Ab (BAF270). Lipo neg ctrl (=cells only treated with transfection agent Lipofectamine which serve as a negative control).

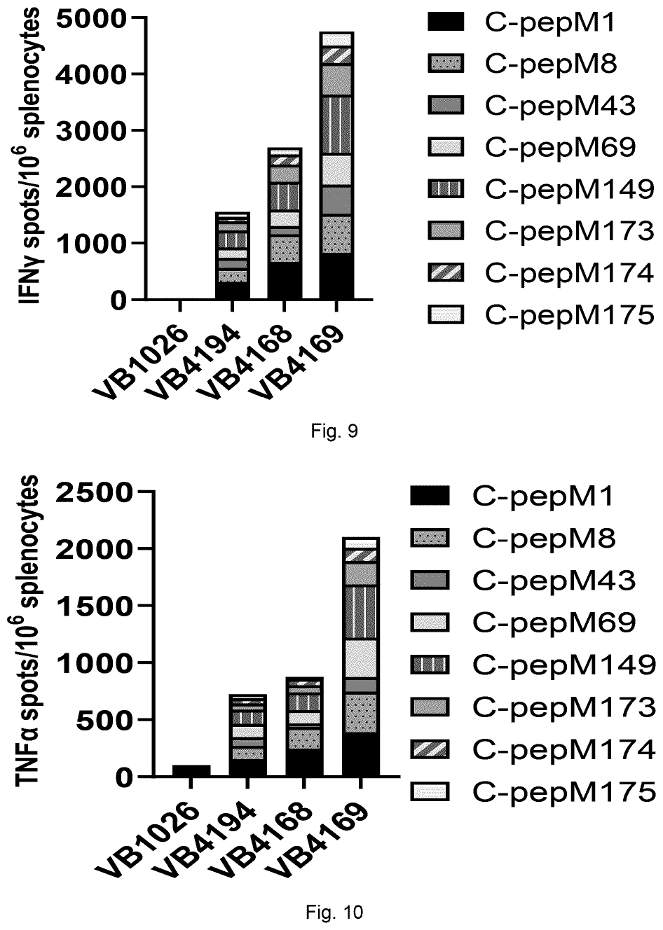

### FIG. 6 Expression and Secretion Levels of Proteins Encoded by a DNA Plasmid

Shows the protein expression and secretion levels of the immunostimulatory compound FLTL3 encoded by the DNA plasmids VB4168, VB4169 and VB4170 detected in the supernatant of HEK293 cells transfected with said DNA plasmids by ELISA using mouse a-human FLT3L capture Ab (MAB608) and mouse a-human FLT3L biotinylated detection Ab (BAF308).

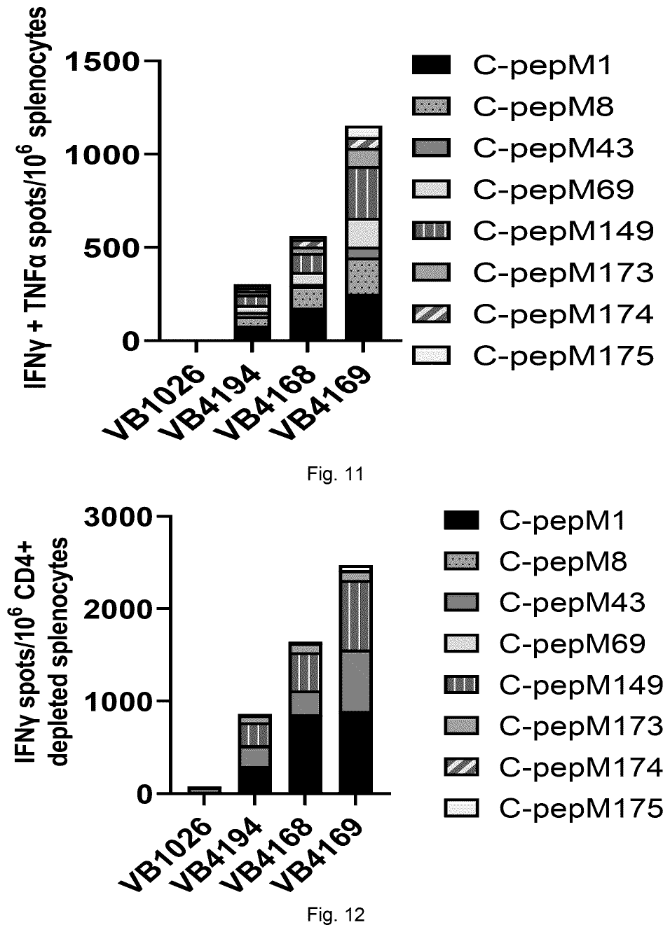

### FIG. 7 Expression and Secretion Levels of Proteins Encoded by a DNA Plasmid

Shows the protein expression and secretion levels of the immunostimulatory compound GM-CSF encoded by the DNA plasmids VB4169 and VB4170 detected in the supernatant of HEK293 cells transfected with said DNA plasmids by ELISA using rat a-mouse GM-CSF capture Ab (MAB415) and goat a-mouse GM-CSF biotinylated detection Ab (BAM215).

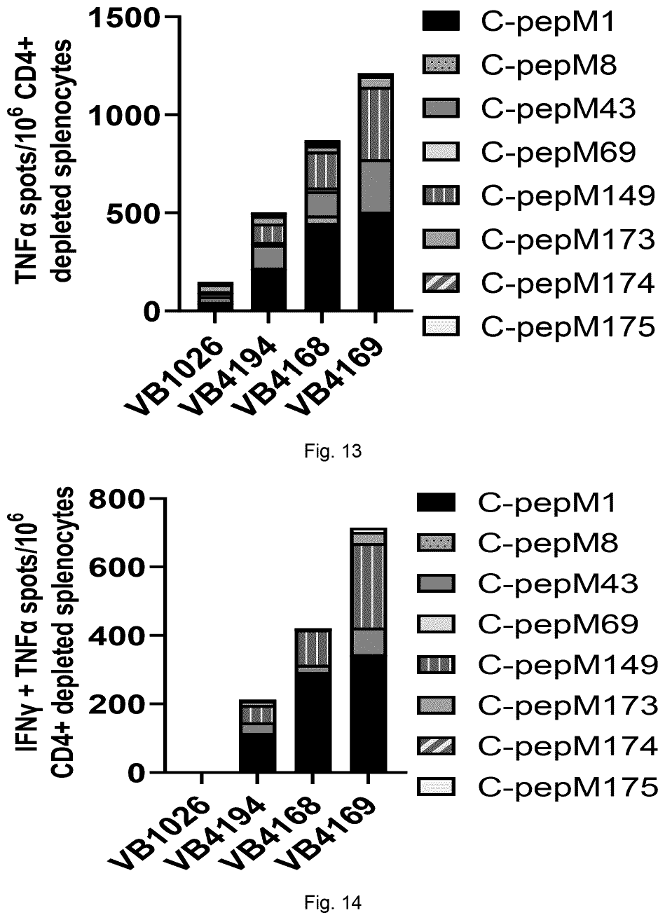

### FIG. 8 Expression and Secretion Levels of Proteins Encoded by a DNA Plasmid

Shows the protein expression and secretion levels of the immunostimulatory compound CCL5 encoded by the DNA plasmid VB4170 detected in the supernatant of HEK293 cells transfected with said DNA plasmid by ELISA using rat a-mouse CCL5 capture Ab (MAB4781) and goat a-mouse CCL5 biotinylated detection Ab (BAF478).

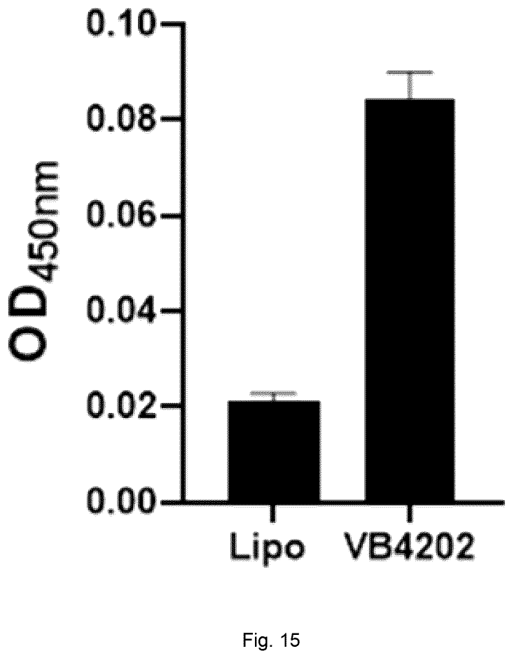

### FIG. 9 Immunogenicity of DNA Plasmids

Shows the immunogenicity of DNA plasmids VB4194, VB4168 and VB4169 in mice administered with these plasmids by way of measuring the IFN-γ secretion from T cells (total T cell response), compared to the negative control VB1026.

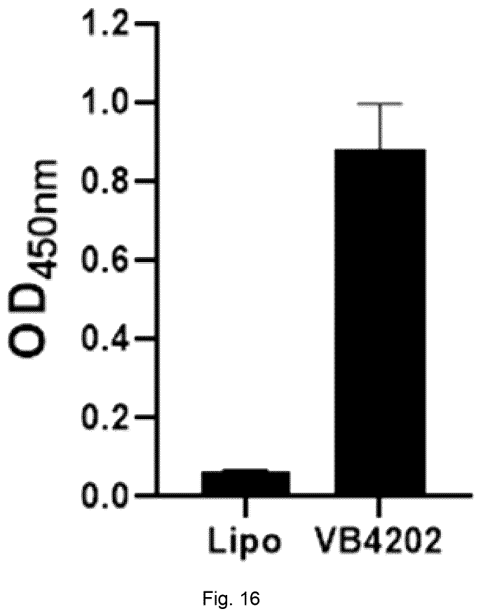

### FIG. 10 Immunogenicity of DNA Plasmids

Shows the immunogenicity of DNA plasmids VB4194, VB4168 and VB4169 in mice administered with these plasmids by way of measuring the TNF-α secretion from T cells (total T cell response), compared to the negative control VB1026.

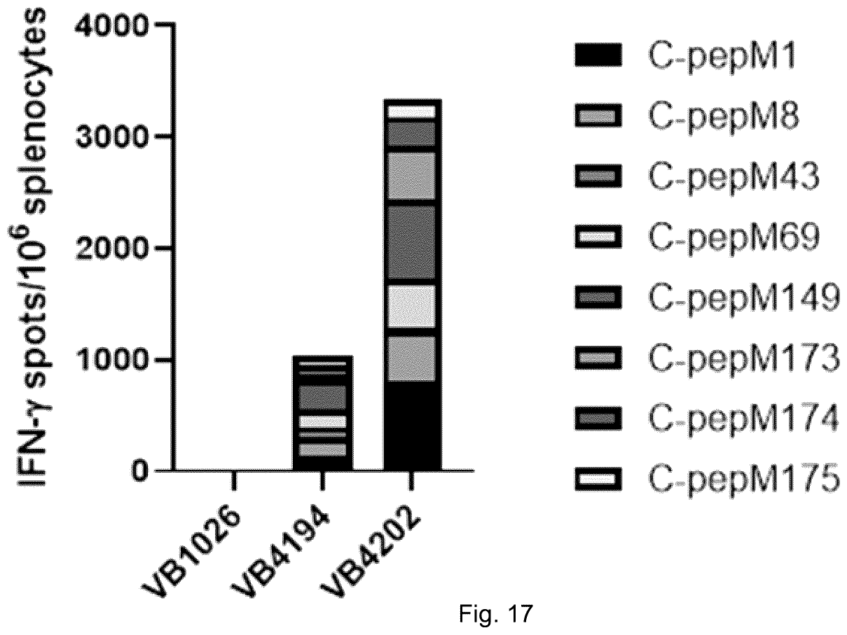

### FIG. 11 Immunogenicity of DNA Plasmids

Shows the immunogenicity of DNA plasmids VB4194, VB4168 and VB4169 in mice administered with these plasmids by way of measuring the IFN-γ+TNF-α co-secretion from T cells (total T cell response), compared to the negative control VB1026.

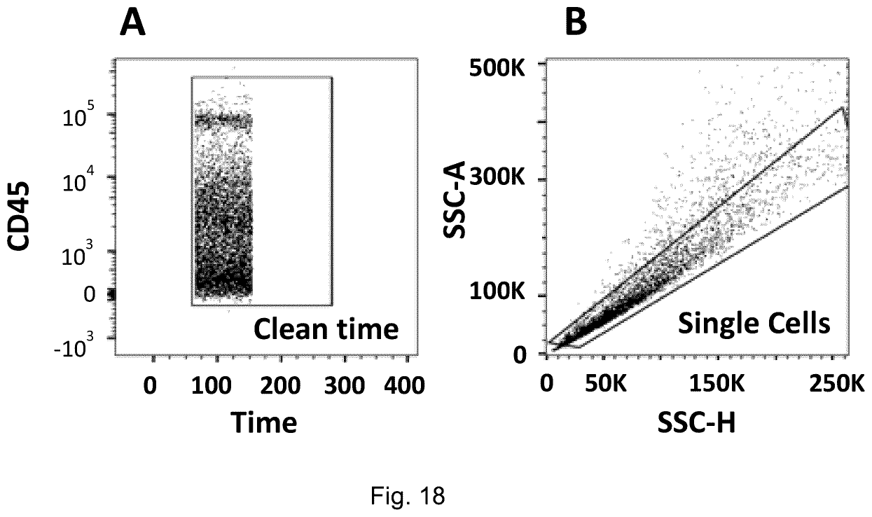

### FIG. 12 Immunogenicity of DNA Plasmids

Shows the immunogenicity of DNA plasmids VB4194, VB4168 and VB4169 in mice administered with these plasmids by way of measuring the IFN-γ secretion from CD8+ T cells (CD4+ T cells depleted samples), compared to the negative control VB1026.

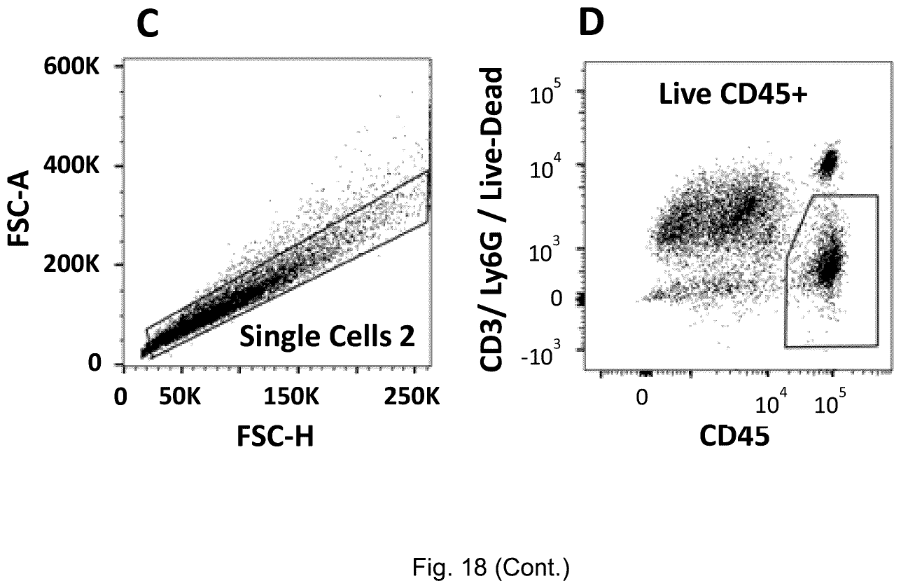

### FIG. 13 Immunogenicity of DNA Plasmids

Shows the immunogenicity of DNA plasmids VB4194, VB4168 and VB4169 in mice administered with these plasmids by way of measuring the TNF-α secretion from CD8+ T cells (CD4+ T cells depleted samples), compared to the negative control VB1026.

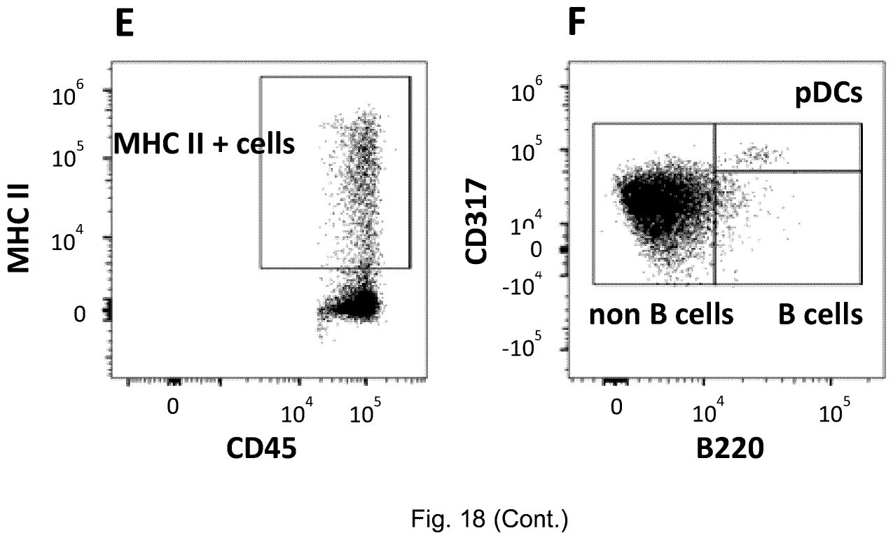

### FIG. 14 Immunogenicity of DNA Plasmids

Shows the immunogenicity of DNA plasmids VB4194, VB4168 and VB4169 in mice administered with these plasmids by way of measuring the IFN-γ+TNF-α secretion from CD8+ T cells (CD4+ T cells depleted samples), compared to the negative control VB1026.

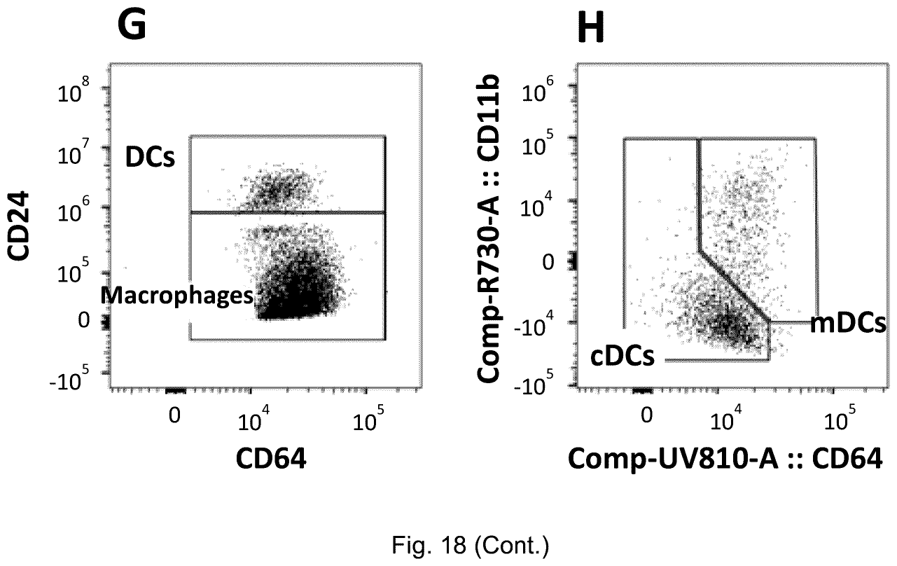

### FIG. 15 Expression and Secretion Levels of Proteins Encoded by a DNA Plasmid

Shows the protein expression and secretion level of the first polypeptide encoded by DNA plasmid VB4202 detected in the supernatant of HEK293 cells transfected with said DNA plasmid by ELISA using mouse a-human IgG CH3 domain capture Ab (MCA878G) and a-human MIP-1α biotinylated capture Ab (BAF270). Lipo (=cells only treated with transfection agent Lipofectamine which serve as a negative control).

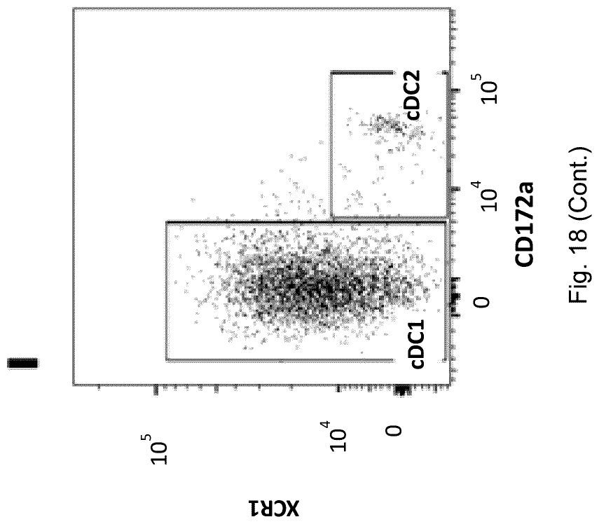

### FIG. 16 Expression and Secretion Levels of Proteins Encoded by a DNA Plasmid

Shows the protein expression and secretion level of the immunostimulatory compound GM-CSF encoded by the DNA plasmid VB4202 detected in the supernatant (diluted 1:1000) of HEK293 cells transfected with said DNA plasmid by ELISA using rat a-mouse GM-CSF capture Ab (MAB415) and goat a-mouse GM-CSF biotinylated detection Ab (BAM215).

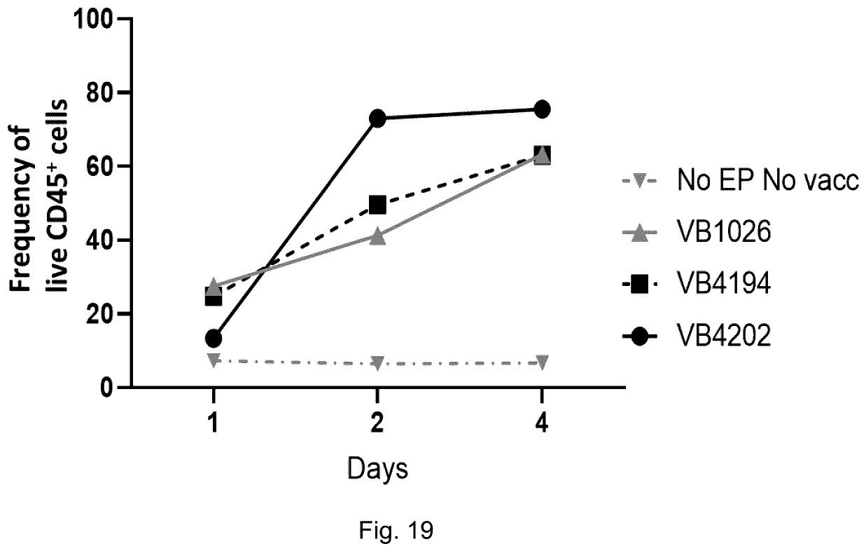

### FIG. 17 Immunogenicity of DNA Plasmids

Shows the immunogenicity of DNA plasmids VB4194 and VB4202 in mice administered with these plasmids by way of measuring the IFN-γ secretion from T cells (total T cell response), compared to the negative control VB1026.

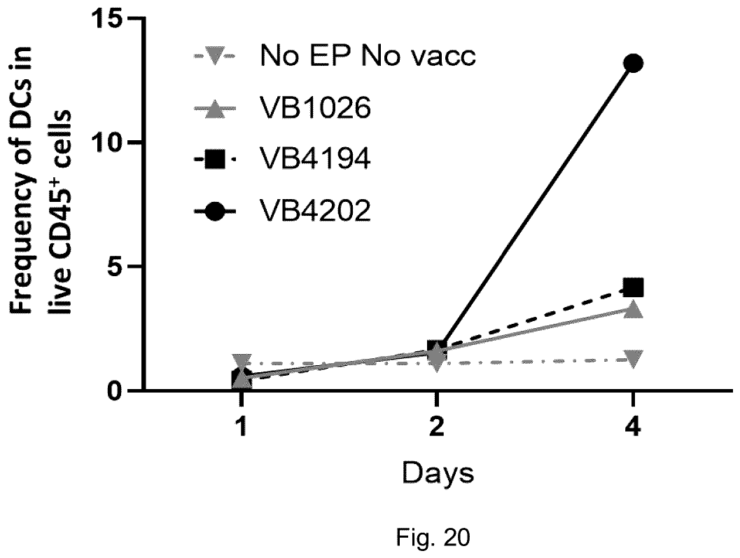

### FIG. 18 Immunogenicity of DNA Plasmids

Shows a flow cytometry assessment used to evaluate dendritic cell (DC) responses on a single cell level in mice administered with DNA plasmids VB1026, VB4194 and VB4202. The Figure shows the gating strategy used to define DCs. A. All events were examined using a time parameter to exclude any fluidics inconsistencies. B. Side scatter (SSC) height and area parameters were used for further exclusion of doublets. C. Forward scatter (FSC) height and area parameters were used for exclusion of doublets. D. Dead cells, neutrophils and T cells were excluded and CD45+ immune cells were used in further analysis. E. MHCII expressing cells were gated and used for further analysis. F. B cells and plasmacytoid (p) DCs were excluded from the analysis. G. DCs were defined as CD24+. H. All DCs were divided in monocyte derived (mo)DC and classical (c) DCs based on CD11b and CD64 expression. I. Classical DCs were divided into cDC1 and cDC2 based on the expression of XCR1 and CD172a markers.

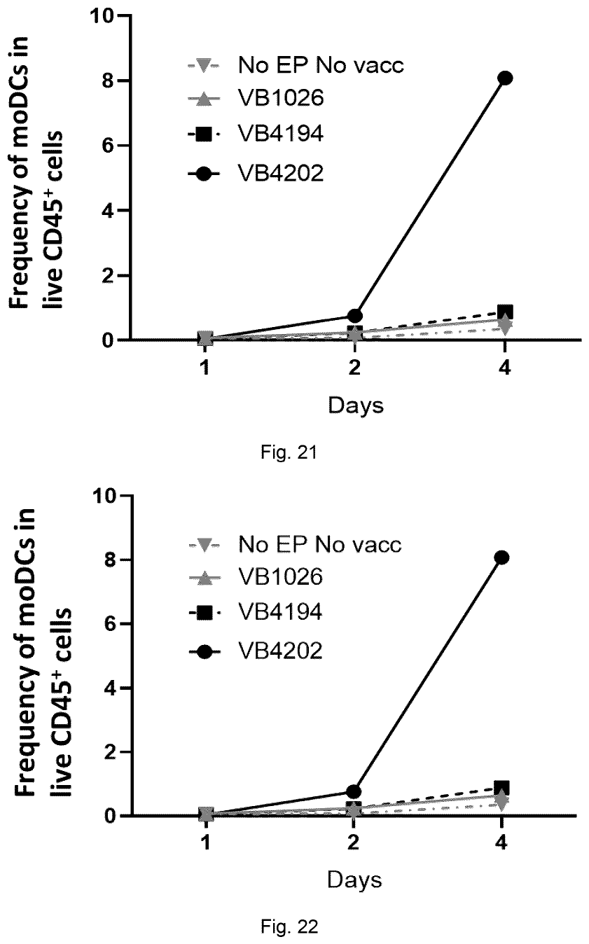

### FIG. 19 Immunogenicity of DNA Plasmids

Shows the proportion of live CD45+ cells at the administration site 1, 2, and 4 days after intramuscular administration with the DNA plasmid VB4202 in comparison to the DNA plasmids VB4194 (comparison) VB1026 (negative control). No EP No vacc refers to results from a group of mice to which no plasmid was administered and no electroporation treatment was carried out.

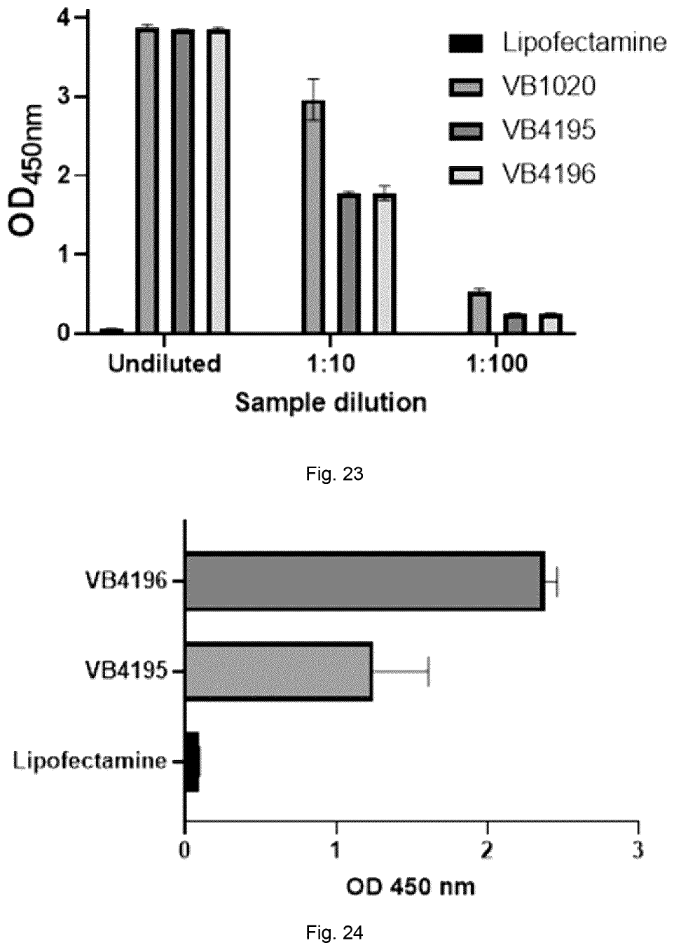

### FIG. 20 Immunogenicity of DNA Plasmids

Shows the proportion of DCs in live CD45+ cells at the administration site 1, 2, and 4 days after intramuscular administration with the DNA plasmid VB4202 in comparison to the DNA plasmids VB4194 (comparison) VB1026 (negative control). No EP No vacc refers to results from a group of mice to which no plasmid was administered and no electroporation treatment was carried out.

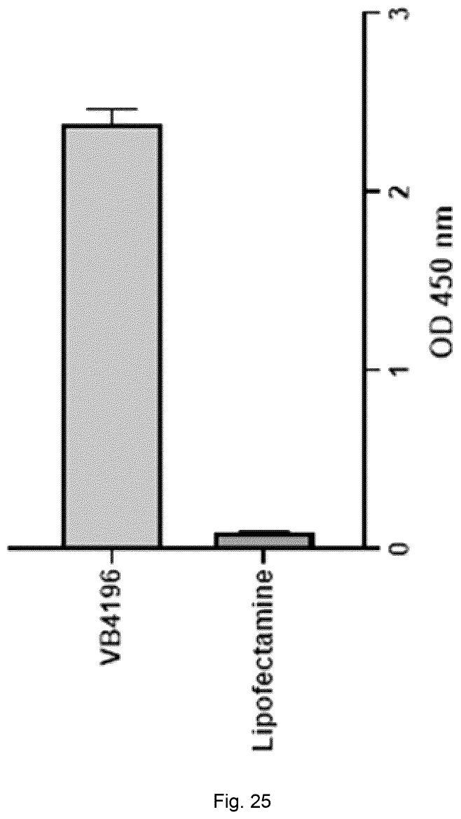

### FIG. 21 Immunogenicity of DNA Plasmids

Shows the proportion of cDC1 cells in live CD45+ cells at the administration site 1, 2, and 4 days after intramuscular administration with the DNA plasmid VB4202 in comparison to the DNA plasmids VB4194 (comparison) VB1026 (negative control). No EP No vacc refers to results from a group of mice to which no plasmid was administered and no electroporation treatment was carried out.

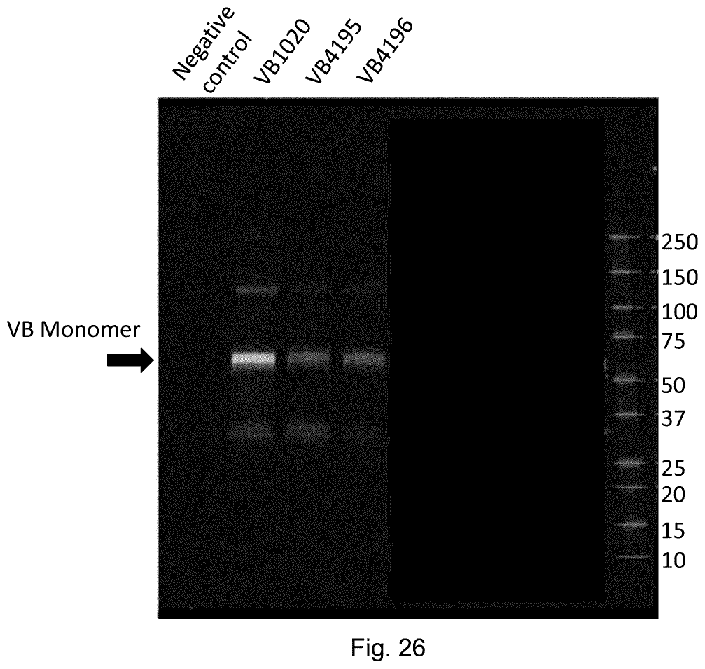

### FIG. 22 Immunogenicity of DNA Plasmids

Shows the proportion of moDC cells in live CD45+ cells at the administration site 1, 2, and 4 days after intramuscular administration with the DNA plasmid VB4202 in comparison to the DNA plasmids VB4194 (comparison) VB1026 (negative control). No EP No vacc refers to results from a group of mice to which no plasmid was administered and no electroporation treatment was carried out.

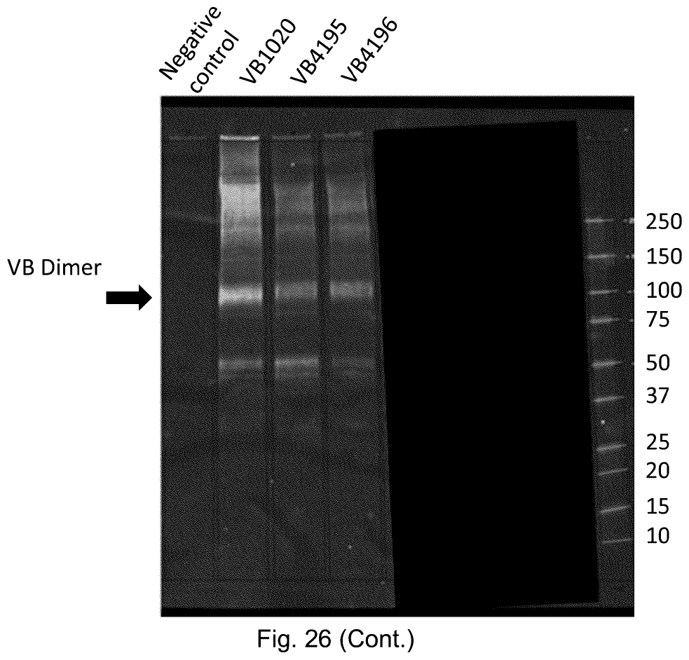

### FIG. 23 Expression and Secretion Levels of Proteins Encoded by a DNA Plasmid

Shows the protein expression and secretion levels of the first polypeptide encoded by DNA plasmids VB1020, VB4195 and VB4196 detected in the supernatant of HEK293 cells transfected with said DNA plasmids by ELISA using mouse a-human IgG CH3 domain capture Ab (MCA878G) and a-human MIP-1α biotinylated capture Ab (BAF270). Lipofectamine (=cells only treated with transfection agent Lipofectamine which serve as a negative control).

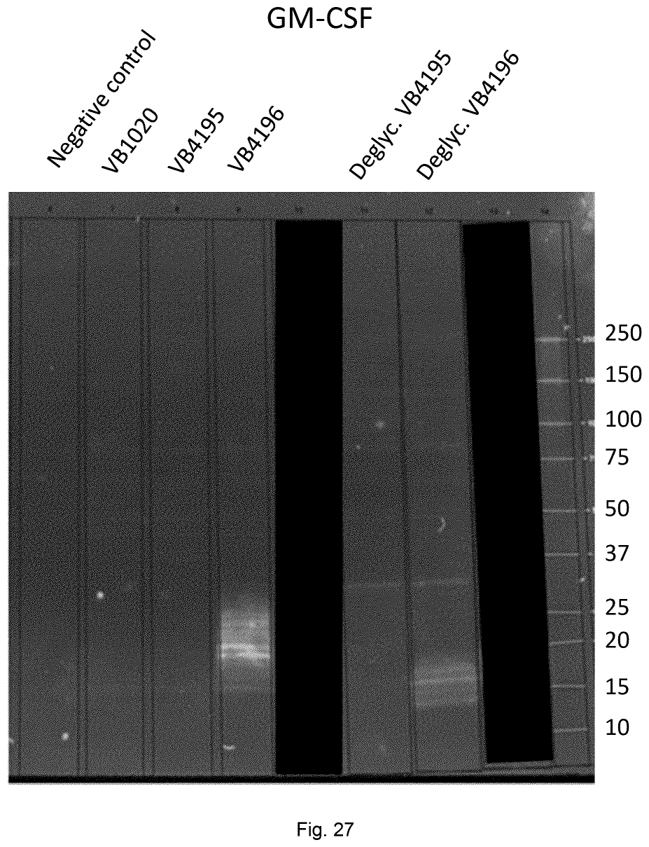

### FIG. 24 Expression and Secretion Levels of Proteins Encoded by a DNA Plasmid

Shows the protein expression and secretion level of the immunostimulatory compound FLT3L encoded by DNA plasmids VB4195 and VB4196 detected in the supernatant (diluted 1:500) of HEK293 cells transfected with said DNA plasmids by ELISA using mouse a-human FLT3L capture Ab (MAB608) and mouse a-human FLT3L biotinylated detection Ab (BAF308). Lipofectamine (=cells only treated with transfection agent Lipofectamine which serve as a negative control), supernatant from cells treated with Lipofectamine only was not diluted for the ELISA.

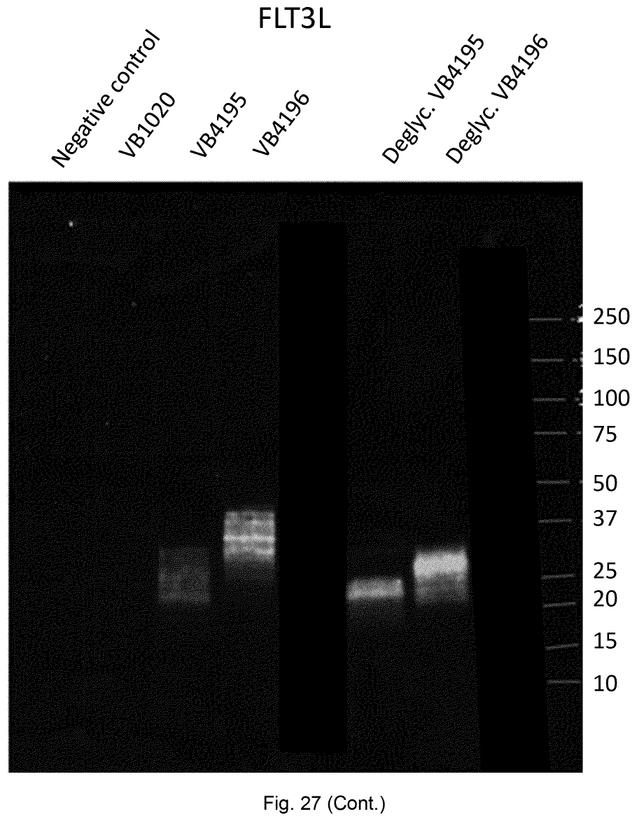

### FIG. 25 Expression and Secretion Levels of Proteins Encoded by a DNA Plasmid

Shows the protein expression and secretion level of the immunostimulatory compound GM-CSF encoded by the DNA plasmid VB4196 detected in the supernatant (diluted 1:500) of HEK293 cells transfected with said DNA plasmid by ELISA using rat a-mouse GM-CSF capture Ab (MAB415) and goat a-mouse GM-CSF biotinylated detection Ab (BAM215). Lipofectamine (=cells only treated with transfection agent Lipofectamine which serve as a negative control), supernatant from cells treated with Lipofectamine only was not diluted for the ELISA.

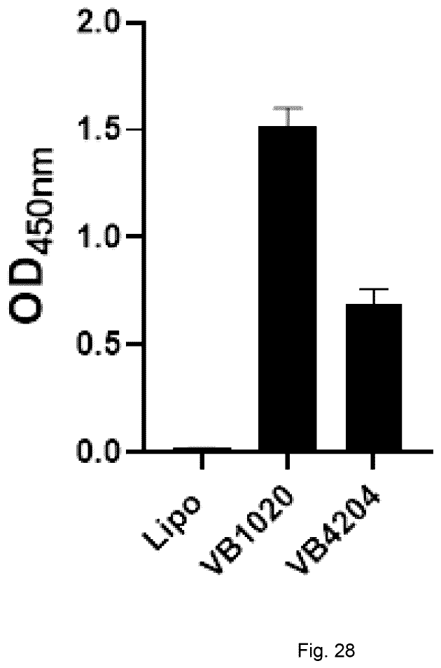

### FIG. 26 Expression and Secretion of Intact Proteins Encoded by a DNA Plasmid

Western blots of non-reduced (left) and reduced (right) supernatant samples of Expi293F cells transfected with DNA plasmids VB1020, VB4195 and VB4196. Primary antibody: goat a-human MIP-1α (BAF270). Secondary antibody: donkey anti-goat, Dylight 550 (SA5-10087). Chemidoc channels Dylight 550 and 650 (for protein standard). Blackened lanes contain samples not relevant for this application.

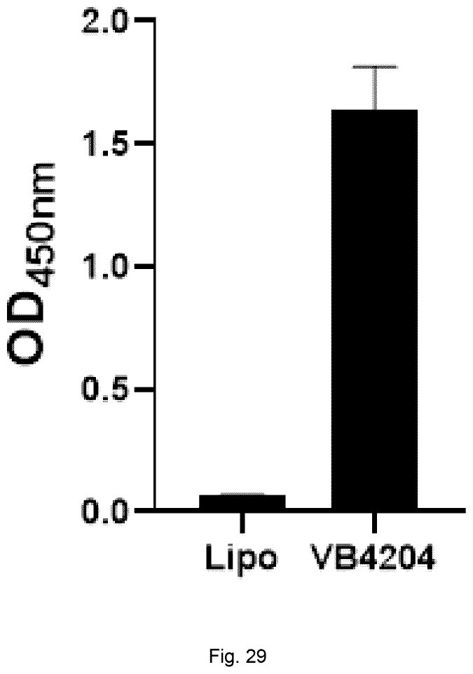

### FIG. 27 Expression and Secretion of Intact Proteins Encoded by a DNA Plasmid

Western blots of reduced supernatant samples (lanes 1-4) and deglycosylated supernatant samples (lanes 5-6) of Expi293F cells transfected with DNA plasmids VB1020, VB4195 and VB4196. Left: Primary antibody goat a-human FLT3L (BAF308). Secondary antibody: donkey anti-goat, Dylight 550 (SA5-10087). Right: Primary antibody goat a-mouse GM-CSF (BAF415). Secondary antibody: donkey anti-goat, Dylight 550 (SA5-10087). Chemidoc channels Dylight 550 and 650 (for protein standard). Blackened lanes contain samples not relevant for this application.

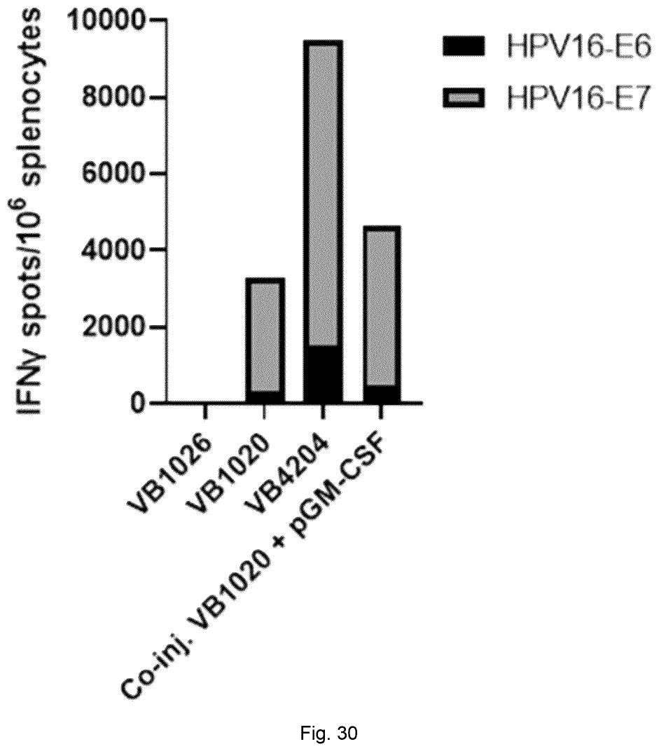

### FIG. 28 Expression and Secretion Levels of Proteins Encoded by a DNA Plasmid

Shows the protein expression and secretion levels of the first polypeptide encoded by DNA plasmids VB1020 and VB4204 detected in the supernatant (diluted 1:10) of HEK293 cells transfected with said DNA plasmids by ELISA using mouse a-human IgG CH3 domain capture Ab (MCA878G) and a-human MIP-1α biotinylated capture Ab (BAF270). Lipo (=cells only treated with transfection agent Lipofectamine which serve as a negative control).

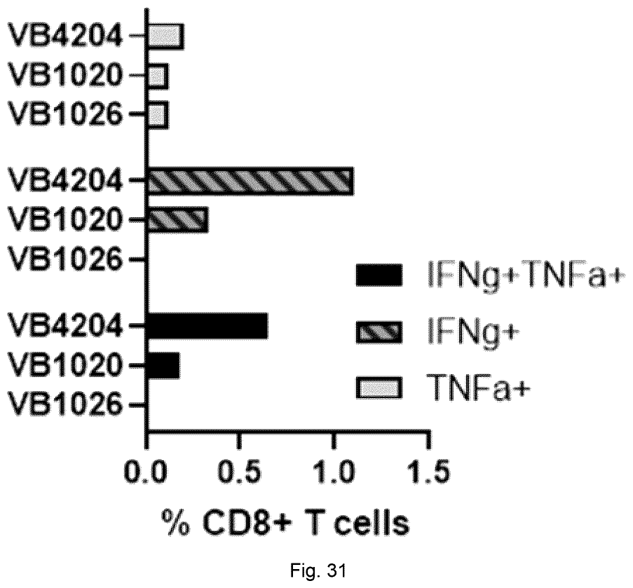

### FIG. 29 Expression and Secretion Levels of Proteins Encoded by a DNA Plasmid

Shows the protein expression and secretion level of the immunostimulatory compound GM-CSF encoded by the DNA plasmid VB4204 detected in the supernatant (diluted 1:1000) of HEK293 cells transfected with said DNA plasmid by ELISA using rat a-mouse GM-CSF capture Ab (MAB415) and goat a-mouse GM-CSF biotinylated detection Ab (BAM215). Lipo (=cells only treated with transfection agent Lipofectamine which serve as a negative control).

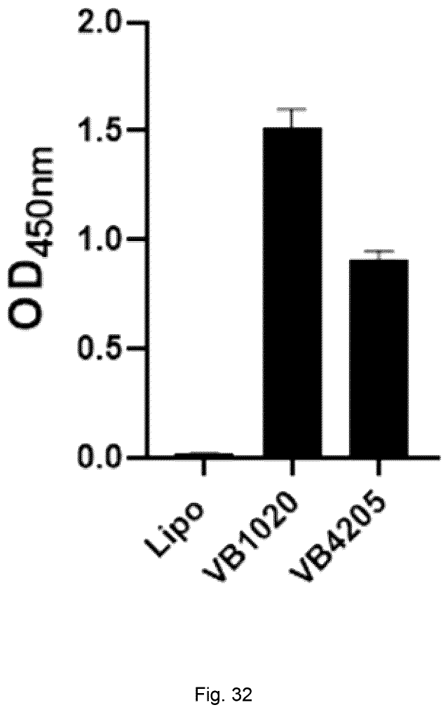

### FIG. 30 Immunogenicity of DNA Plasmids

Shows the immunogenicity of DNA plasmids VB1020 and VB4204 in mice administered with these plasmids and the immunogenicity of co-administered DNA plasmids VB1020 plus pGM-CSF by way of measuring the IFN-γ secretion from T cells (total T cell response), compared to the negative control VB1026.

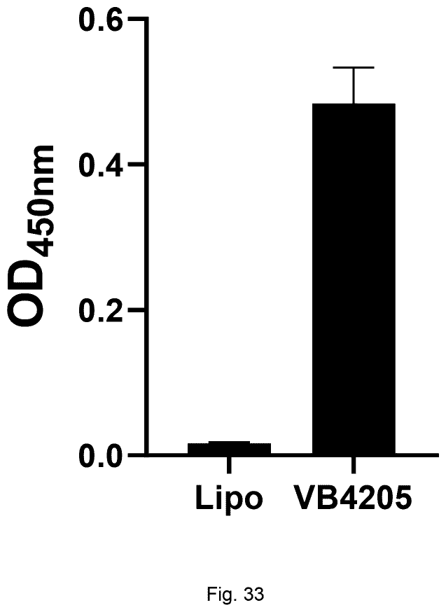

### FIG. 31 Immunogenicity of DNA Plasmids

Shows the percentage of CD8+ T cells secreting IFN-γ, TNF-α, or co-secreting TNF-α and IFN-γ in splenocytes from mice administered with DNA plasmids VB1020 and VB4204, compared to the negative control VB1026

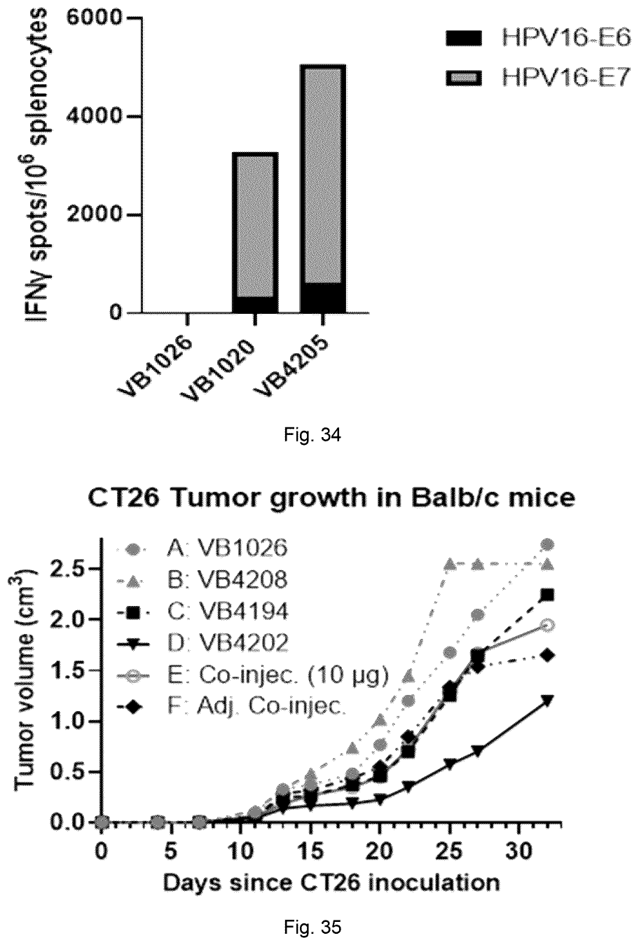

### FIG. 32 Expression and Secretion Levels of Proteins Encoded by a DNA Plasmid

Shows the protein expression and secretion levels of the first polypeptide encoded by DNA plasmids VB1020 and VB4205 detected in the supernatant (diluted 1:10) of HEK293 cells transfected with said DNA plasmids by ELISA using mouse a-human IgG CH3 domain capture Ab (MCA878G) and a-human MIP-1α biotinylated capture Ab (BAF270). Lipo (=cells only treated with transfection agent Lipofectamine which serve as a negative control).

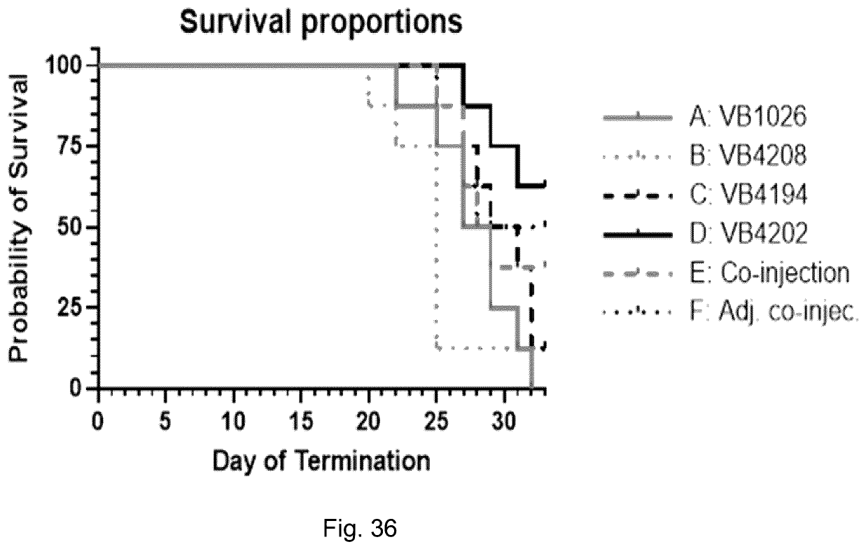

### FIG. 33 Expression and Secretion Levels of Proteins Encoded by a DNA Plasmid

Shows the protein expression and secretion level of the immunostimulatory compound CCL5 encoded by the DNA plasmid VB4205 detected in the supernatant (diluted 1:500) of HEK293 cells transfected with said DNA plasmid ELISA using rat a-mouse CCL5 capture Ab (MAB4781) and goat a-mouse CCL5 biotinylated detection Ab (BAF478). Lipo (=cells only treated with transfection agent Lipofectamine which serve as a negative control).

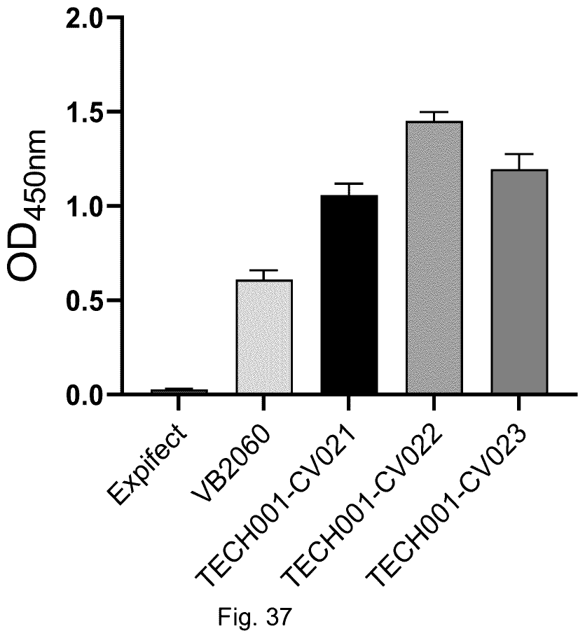

### FIG. 34 Immunogenicity of DNA Plasmids

Shows the immunogenicity of DNA plasmids VB1020 and VB4205 in mice administered with these plasmids by way of measuring the IFN-γ secretion from T cells (total T cell response), compared to the negative control VB1026.

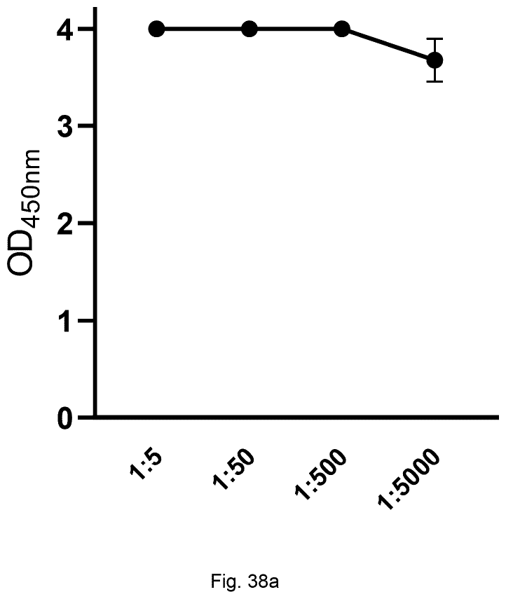

### FIG. 35 Immunogenicity of DNA Plasmids

Shows the CT26 tumor growth in in BALB/c mice (n=8/group) inoculated with 1×10⁵ CT26 tumor cells on DO, followed by administration with DNA plasmids VB4194, VB4208, VB4202, and pGM-CSF+VB4194 (co-injection) on D4 and D11 compared to the negative control VB1026.

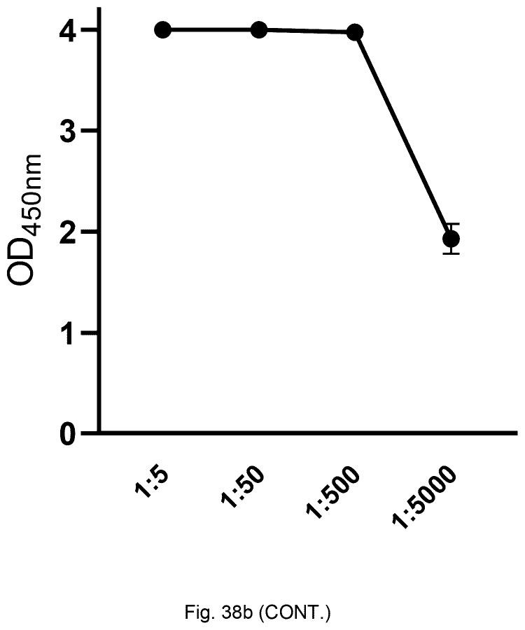

### FIG. 36 Immunogenicity of DNA Plasmids

Shows the probability of survival of BALB/c mice (n=8/group) inoculated with 1×10⁵ CT26 tumor cells on DO, followed by administration with DNA plasmids VB4194, VB4208, VB4202, and pGM-CSF+VB4194 (co-injection) on D4 and D11 compared to the negative control VB1026.

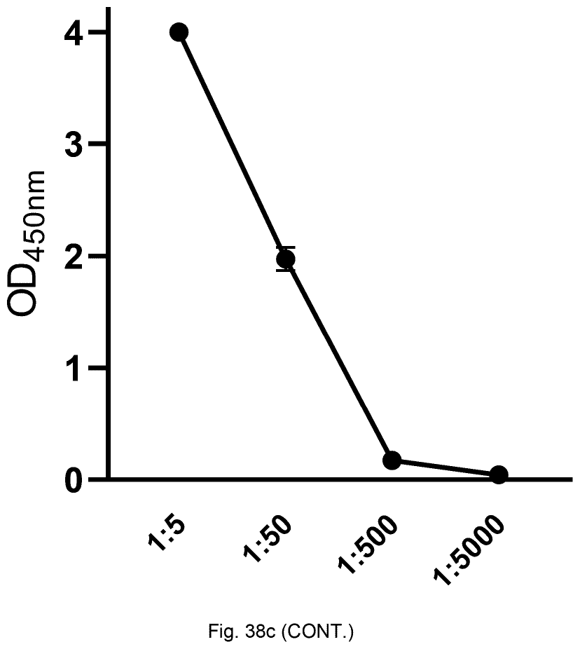

### FIG. 37 Expression and Secretion Levels of Proteins Encoded by a DNA Plasmid

Shows the secretion of the first polypeptide encoded by DNA plasmids VB2060, TECH001-CV021, TECH001-CV022 and TECH001-CV023 detected in the supernatants of Expi293F cells transfected with said DNA plasmids by ELISA. The supernatants were diluted 1:1500 and ELISA was performed using mouse a-human IgG CH3 domain capture Ab (MCA878G) and a-human MIP-1α biotinylated capture Ab (BAF270). Expifect (=cells only treated with transfection agent Expifectamine which serve as a negative control).

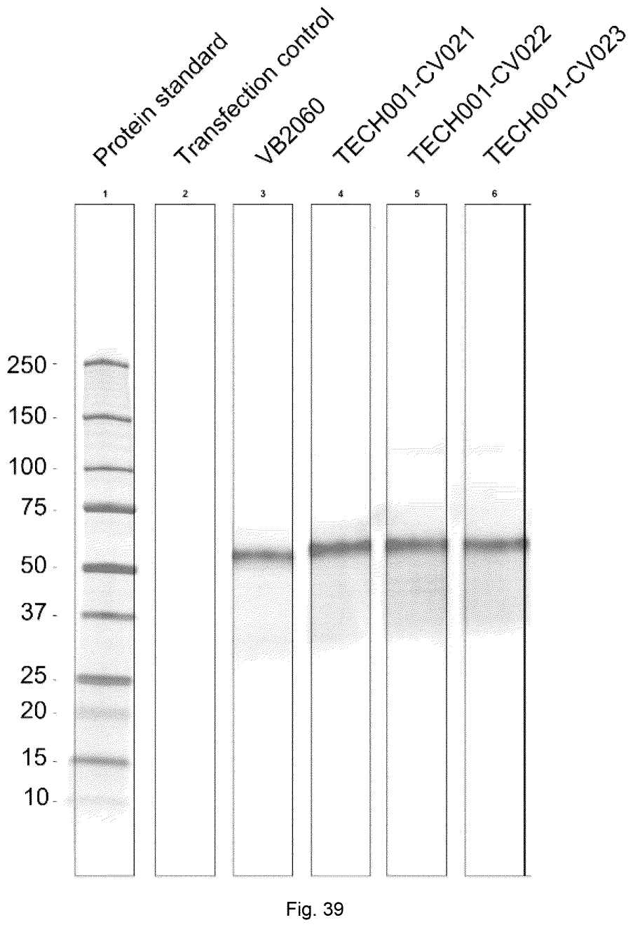

### FIG. 38 Expression and Secretion Levels of Proteins Encoded by a DNA Plasmid

Shows the protein expression and secretion level of the immunostimulatory compound GM-CSF (38a: capture Ab MAB608, detection Ab BAF308), IL-12 (38b: capture Ab MAB419, detection Ab BAF419), and IL-21 (38c: capture Ab AF594, detection Ab BAF594) encoded by the DNA plasmids TECH001-CV021, TECH001-CV022 and TECH001-CV023, respectively, detected in the supernatant of Expi293F cells transfected with said DNA plasmids by ELISA.

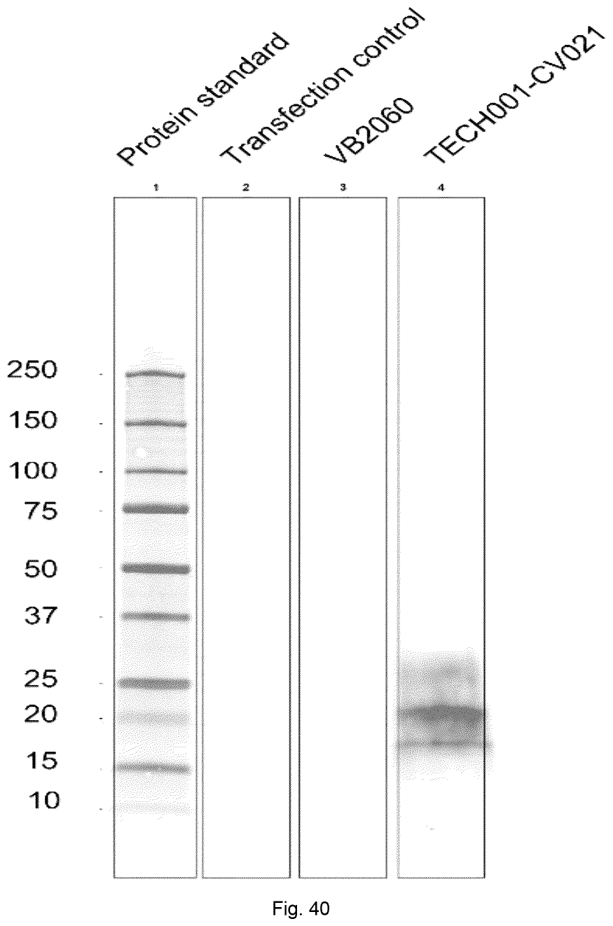

### FIG. 39 Expression and Secretion of Intact Proteins Encoded by a DNA Plasmid

Western blot shows secretion of the first polypeptide. Reduced supernatant samples from transfection control, VB2060, TECH001-CV021, TECH001-CV022 and TECH001-CV023. Primary antibody: goat anti-human MIP-1α (AF270). Secondary antibody: donkey anti-goat, Dylight 800 (SA5-10092). Chemidoc channels Dylight 800 and 650 (for protein standard).

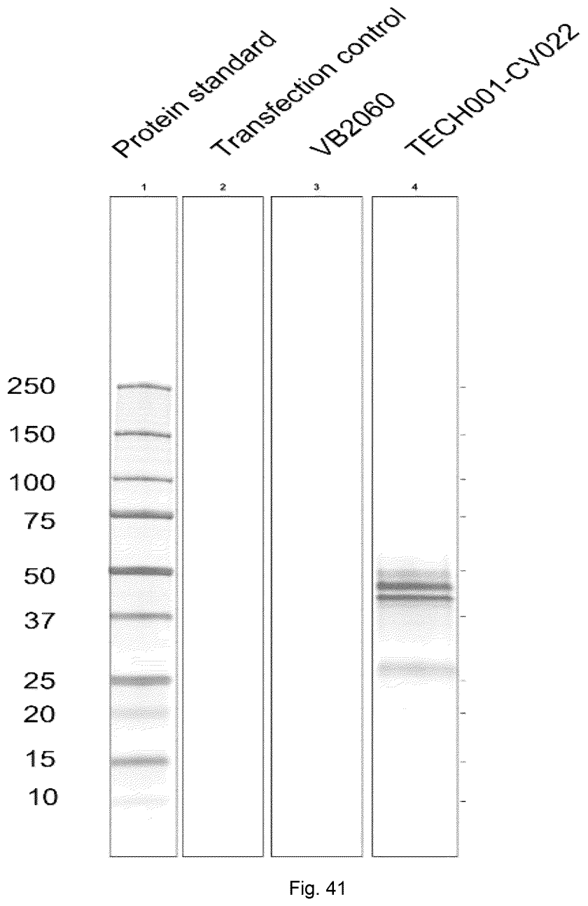

### FIG. 40 Expression and Secretion of Intact Proteins Encoded by a DNA Plasmid

Western blot shows the secretion of the immunostimulatory compound GM-CSF encoded by TECH001-CV021. Reduced supernatant samples from transfection control, VB2060 and TECH001-CV021. Primary antibody: goat anti-mouse GM-CSF (BAF415). Secondary antibody: donkey anti-goat, Dylight 800 (SA5-10092). Chemidoc channels Dylight 800 and 650 (for protein standard).

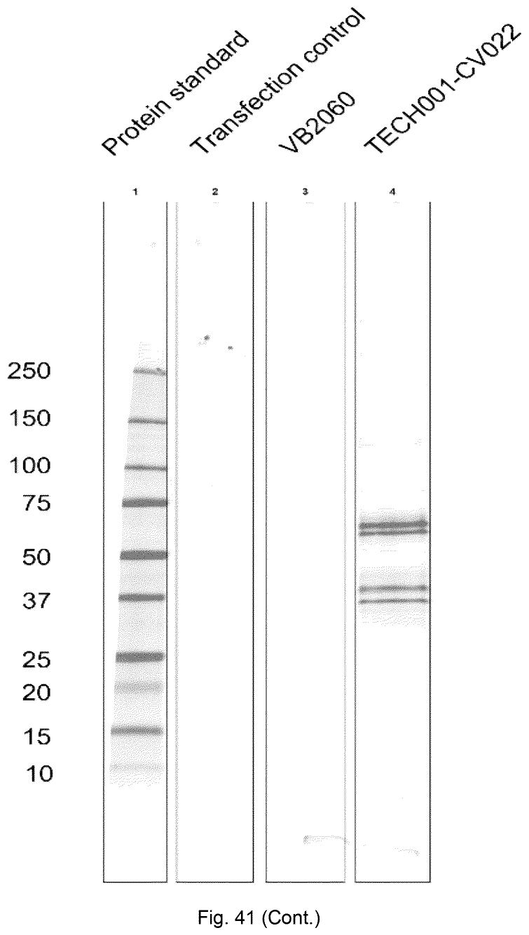

### FIG. 41 Expression and Secretion of Intact Proteins Encoded by a DNA Plasmid

Western blots show the secretion of the immunostimulatory compound IL-12 encoded by TECH001-CV022. Reduced supernatant samples (left panel) and non-reduced supernatant samples (right panel) from transfection control, VB2060 and TECH001-CV022. Primary antibody: goat anti-mouse IL-12 (BAF419). Secondary antibody: donkey anti-goat, Dylight 800 (SA5-10092). Chemidoc channels Dylight 800 and 650 (for protein standard).

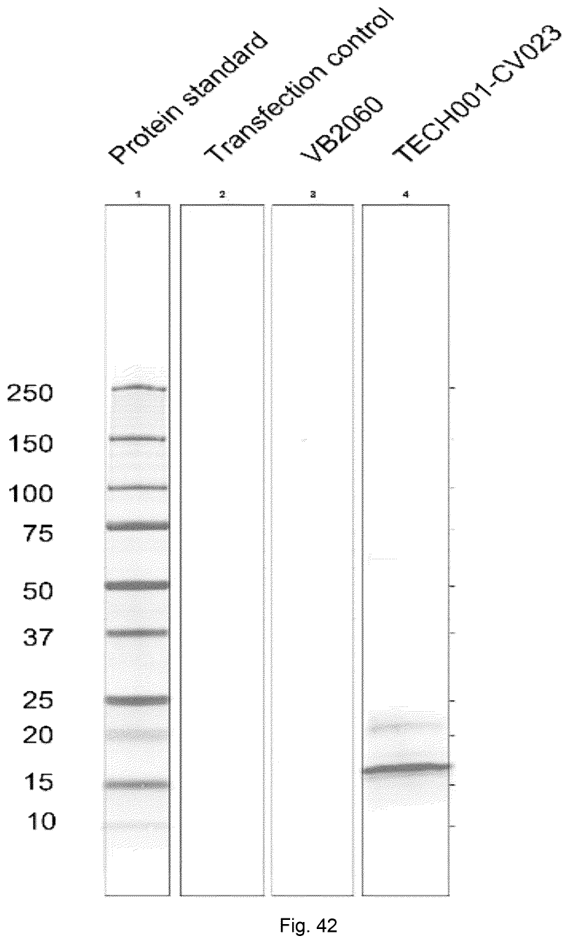

### FIG. 42 Expression and Secretion of Intact Proteins Encoded by a DNA Plasmid

Western blot shows the secretion of the immunostimulatory compound IL-21 encoded by TECH001-CV023. Reduced supernatant samples from transfection control, VB2060 and TECH001-CV023. Primary antibody: goat anti-mouse IL-12 (BAF594). Secondary antibody: donkey anti-goat, Dylight 800 (SA5-10092). Chemidoc channels Dylight 800 and 650 (for protein standard).

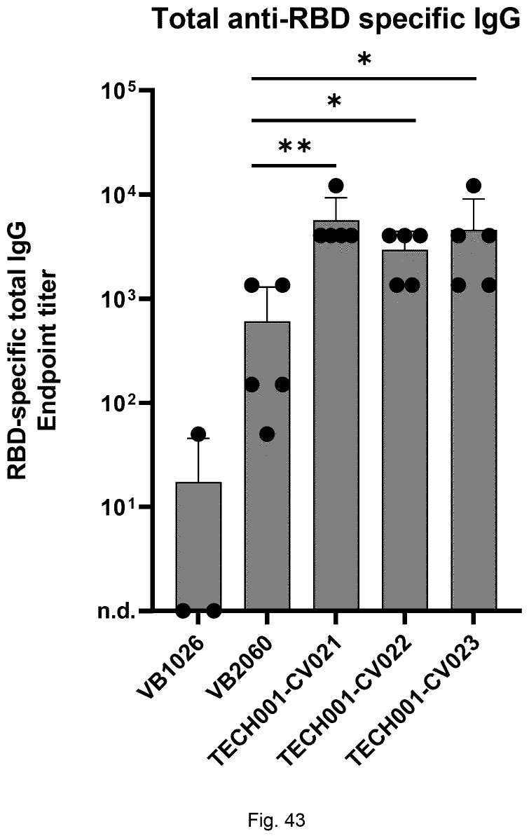

### FIG. 43 Immunogenicity of DNA Plasmids

Shows the immunogenicity of DNA plasmids VB2060, TECH001-CV021, TECH001-CV022 and TECH001-CV023 in mice administered with these plasmids by way of measuring total IgG antibodies binding the RBD protein compared to the negative control VB1026. Individual mice and mean±SEM are shown, 5 mice per group. *(p<0.05), **(p<0.01), two-tailed Mann-Whitney test.

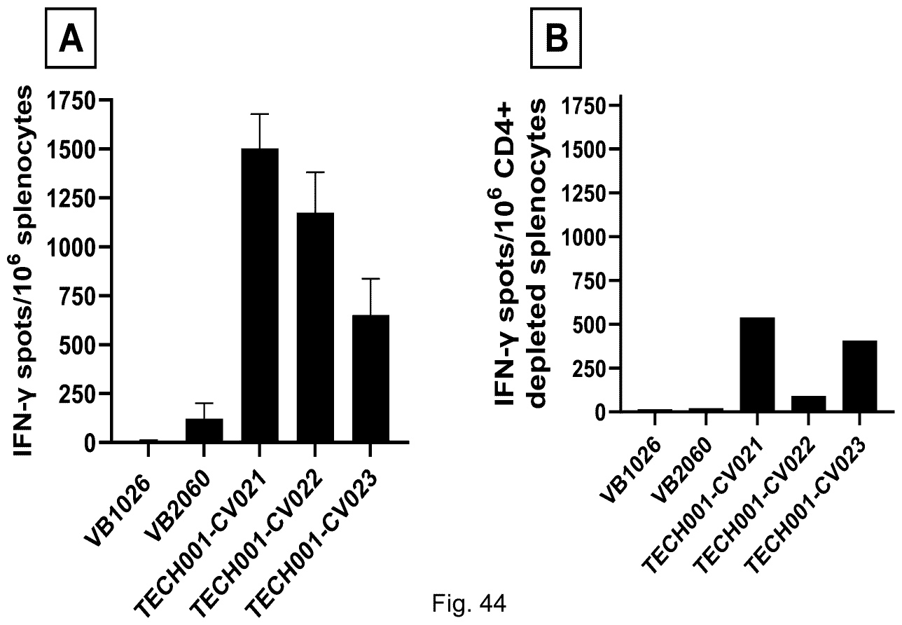

### FIG. 44 Immunogenicity of DNA Plasmids

A) Shows the immunogenicity of DNA plasmids VB2060, TECH001-CV021, TECH001-CV022 and TECH001-CV023 in mice administered with these plasmids by way of measuring the IFN-γ secretion from T cells (total T cell response) compared to the negative control VB1026. B) Shows the immunogenicity of DNA plasmids VB2060, TECH001-CV021, TECH001-CV022 and TECH001-CV023 in mice administered with these plasmids by way of measuring the IFN-γ secretion from CD8+ T cells (CD4+ T cells depleted samples), compared to the negative control VB1026.

### DETAILED DESCRIPTION

The first polypeptide and/or the multimeric protein will herein also be referred to as a “construct”. The first polypeptides/multimeric proteins described herein are generally immunogenic constructs.

An “immunogenic construct” is one that elicits an immune response, particularly when administered to a subject in a form suitable for administration and in an amount effective to elicit the immune response (i.e. an immunologically effective amount).

A “subject” is an animal, e.g. a mouse, or a human, preferably a human. The terms “mouse”, “murine” and “m” are used interchangeably herein to denote a mouse or refer to a mouse. The terms human and “h” are used interchangeably herein to denote a human or refer to a human. A subject may be a patient, i.e. a human suffering from a disease who is in need of a therapeutic treatment, or it may be a subject in need of prophylactic treatment, e.g., from being infected with an infectious disease, or it may be a subject suspected of suffering from a disease. The terms “subject” and “individual” are used interchangeably herein.

A “disease” is an abnormal medical condition that is typically associated with specific signs and symptoms in a subject being affected by the disease.

An “infectious disease” is a disease caused by one or more pathogens, including viruses, bacteria, fungi and parasites.

A “cancer” refers to a broad group of various diseases characterized by the uncontrolled growth of abnormal cells in the body. A “cancer” or “cancer tissue” includes a tumor, and as used herein, encompasses both a solid tumor as well as tumor cells found in a bodily fluid such as blood, and includes metastatic cancer. Unregulated cell division and growth results in the formation of malignant tumors that can invade neighboring tissues and can also metastasize to distant parts of the body through the lymphatic system or bloodstream. Following metastasis, the distal tumors can be said to be “derived from” a pre-metastasis tumor.

A “treatment” is a prophylactic treatment or a therapeutic treatment.

A “prophylactic treatment” is a treatment administered to a subject who does not (or not yet) display signs or symptoms of, or displays only early signs or symptoms of, a disease, such that treatment is administered for the purpose of preventing or decreasing the risk of developing the disease and/or symptoms associated with the disease. A prophylactic treatment functions as a preventative treatment against a disease, or as a treatment that inhibits or reduces further development or enhancement of the disease and/or its associated symptoms. The terms prophylactic treatment, prophylaxis and prevention are used interchangeably herein.

A “therapeutic treatment” is a treatment administered to a subject who displays symptoms or signs of a disease, in which treatment is administered to the subject for the purpose of diminishing or eliminating those signs or symptoms or for the purpose of delaying or stopping disease progression.

A “T cell epitope” as used herein refers to a discrete, single T cell epitope or a part or region of an antigen containing multiple T cell epitopes, e.g. multiple minimal T cell epitopes, such as a hotspot

A “nucleotide sequence” is a sequence consisting of nucleotides. The terms “nucleotide sequence” and “nucleic acid sequence” are used interchangeably herein.

The one or more immunostimulatory compounds enhance the effect of the first polypeptide/multimeric protein. The advantage of the present invention is that by co-expressing the first polypeptide and the one or more immunostimulatory compounds from a single vector, e.g. a DNA plasmid, only such single vector needs to be administered to a subject. Hence, for enhancing the effect of the first polypeptide/multimeric protein, it is not required to produce and administer additional vectors encoding immunostimulatory compounds or to co-administer such compounds in the form of proteins or peptides, thereby reducing the production costs and streamlining drug production. Administration of a single drug product may also contribute to increased patient acceptance of therapy and make handling of the drug product, e.g. reconstitution and administration to the patient, easier for health care professionals.

Further, without wishing to be bound by the theory, the co-expression may also have marked advantages on the cellular level. When transfecting with a vector, the vector will hit a range of cells. Successful uptake and functional initiation of transcription and translation is, to some extent, a random process. When transfecting with two different vectors, it cannot be controlled in which cells the proteins encoded by these vectors will be expressed. For transfections aiming at secretion of proteins to the blood stream, this spatial distribution is not a concern. In the present invention, the vector of the invention produces different proteins. When the vector encoding the construct is administered intramuscularly, the construct is secreted from muscle cells and it is delivered to neighbouring antigen-presenting cells. Since the immunostimulatory compound is expressed in and secreted from the same muscle cell, it can stimulate the same antigen-presenting cell and thereby directly affect said antigen-presenting cell. As an example, if the antigen-presenting cell is a dendritic cell, the immunostimulatory compound can promote the attraction, activation and maturation of the dendritic cell.

The section headings used herein are for organizational purposes only and are not to be construed as limiting the subject matter described.

### Vector

The vectors of the invention may be any molecules which are suitable to carry foreign nucleic acid sequences, such as DNA or RNA, into a cell, where they can be expressed, i.e. expression vectors.

In one embodiment, the vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In another embodiment, the vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In a preferred embodiment, the vector is a DNA vector, more preferably a DNA plasmid.

### DNA Plasmids

A plasmid is a small, extrachromosomal DNA molecule within a cell that is physically separated from chromosomal DNA and can replicate independently. Plasmids are mostly found as small circular, double-stranded DNA molecules in bacteria; however, plasmids are sometimes present in archaea and eukaryotic organisms. Artificial plasmids are widely used as vectors in molecular cloning, serving to deliver and ensure high expression of recombinant DNA sequences within host organisms. Plasmids comprise several important features, including a feature for selection of cells comprising the plasmid, such as for example a gene for antibiotic resistance, an origin of replication, a multiple cloning site (MCS) and promoters for driving the expression of the inserted gene(s) of interest.

Generally, promoters are sequences capable of attracting initiation factors and polymerases to the promoter, so that a gene is transcribed. Promoters are located near the transcription start sites of genes, upstream on the DNA. Promoters can be about 100-1000 base pairs long. The nature of the promoter is usually dependent on the gene and product of transcription and type or class of RNA polymerase recruited to the site. When the RNA polymerase reads the DNA of the plasmid, an RNA molecule is transcribed. After processing, the mRNA will be able to be translated numerous times, and thus result in many copies of the proteins encoded by the genes of interest, when the ribosome translates the mRNA into protein. Generally, the ribosome facilitates decoding by inducing the binding of complementary tRNA anticodon sequences to mRNA codons. The tRNAs carry specific amino acids that are chained together into a polypeptide as the mRNA passes through and is “read” by the ribosome. Translation proceeds in three phases, initiation, elongation and termination. Following the translation process, the polypeptide folds into an active protein and performs its functions in the cell or is exported from the cell and performs its functions elsewhere, sometimes after a considerable number of posttranslational modifications.

When a protein is destined for export out of the cell, a signal peptide directs the protein into the endoplasmic reticulum, where the signal peptide is cleaved off and the protein is transferred to the cell periphery after translation has terminated.

The DNA plasmid of the present invention is not limited to any specific plasmid, the skilled person will understand that any plasmid with a suitable backbone can be selected and engineered by methods known in the art to comprise the elements and units of the present disclosure.

### Co-Expression

The vectors of the present disclosure co-express several proteins. Such vectors (and plasmids) are also referred to as multicistronic or polycistronic vectors (and multicistronic or polycistronic plasmids). The skilled person knows how to engineer a vector to comprise sequences coding for these several proteins and can select different means and use different techniques known in the art to ensure that these proteins are co-expressed from one vector as separate proteins.

Hence, the skilled person can construct the vectors of the invention, co-expressing different proteins, i.e. a first polypeptide and one or more immunostimulatory compounds.

In a preferred embodiment, the vectors of the invention comprise one or more co-expression elements, i.e. nucleic acid sequences which allow for the co-expression of the first polypeptide and the one or more immunostimulatory compounds from the same vector.

In one embodiment of the present disclosure, the vector comprises a co-expression element (or more than one co-expression elements), which causes that the first polypeptide and the one or more immunostimulatory compounds are transcribed on a single transcript but independently translated into the first polypeptide and the one or more immunostimulatory compounds. Hence, the presence of the co-expression element results in a final production of separate translation products.

### IRES

In one embodiment of the present disclosure, the co-expression element is an IRES element, the concept of which is illustrated in FIG. 1. An internal ribosome entry site, abbreviated IRES, is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. By placing an IRES element between two coding regions, the initiation complex can be assembled at this site and allow for translation of the downstream coding region. Hence, in an embodiment of the present disclosure, the vector comprises an IRES and one transcript is produced from the vector, which subsequently is translated into separate proteins.

The IRES element allows the co-expression of the first polypeptide and the one or more immunostimulatory compounds under the control of the same promoter. The promoter directs the transcription of a single mRNA containing coding regions for the nucleic acid sequence encoding the first polypeptide and the nucleic acid sequences encoding the one or more immunostimulatory compounds. If more than one immunostimulatory compound is expressed from the vector of the invention, an IRES element needs to be present in the vector of the invention upstream of each nucleic acid sequence encoding an immunostimulatory compound. Alternatively, another type of co-expression element may be used if more than one immunostimulatory compound is expressed from the vector of the invention.

The IRES elements for use in the vector of the invention may be derived from viral genomes or from cellular mRNA. Vectors comprising IRES elements, such as DNA plasmids, are commercially available.

### 2A Self-Cleaving Peptides

In another embodiment of the present disclosure, the co-expression element is a nucleic acid sequence encoding a 2A self-cleaving peptide (or short “2A peptide”), the concept of which is illustrated in FIG. 2.

In the context of this application, the terms “2A self-cleaving peptide” and “2A peptide” are used for a peptide encoded by a nucleic acid sequence that, when positioned between two coding regions, cause the transcription of the two coding regions as a single transcript, but its translation into two separate peptide chains. Generally, when the ribosome translates mRNA, amino acids are covalently bonded in an N-terminal to C-terminal fashion. The presence of a nucleic acid sequence encoding a 2A self-cleaving peptide results in two separate peptide chains because the ribosome skips the synthesis of a peptide bond at the C-terminus of the 2A peptide. 2A self-cleaving peptides are typically 18-22 amino acids long and often comprise the consensus sequence DXEXNPGP (SEQ ID NO: 50), wherein X can be any amino acid.

In one embodiment of the present invention, the ribosome skips the peptide bond between a glycine and a proline residue found on the C-terminus of the 2A self-cleaving peptide, meaning that the upstream gene product will have a few additional amino acid residues added to the end, while the downstream gene product will start with a proline.

In one embodiment, the 2A self-cleaving peptide is an 18-22 amino acid long sequence comprising the consensus sequence DXEXNPGP (SEQ ID NO: 50), wherein X can be any amino acid.

Thus, also the 2A self-cleaving peptide allows for the co-expression of the first polypeptide and the one or more immunostimulatory compounds under the control of the same promoter. As with the IRES element, if more than one immunostimulatory compound is expressed from the vector of the invention, a nucleic acid sequence encoding a 2A peptide needs to be present in the vector upstream of each nucleic acid sequence encoding an immunostimulatory compound. As an example, the vector comprises a first nucleic acid sequence encoding a first polypeptide, a second nucleic acid sequence encoding a first immunostimulatory compound and a third nucleic acid sequence encoding a second immunostimulatory compound. The vector may comprise a nucleic acid sequence encoding a T2A peptide between the first and the second nucleic acid sequence and a nucleic acid sequence encoding a P2A peptide between the second and the third nucleic acid sequence. Alternatively, another type of co-expression element may be used if more than one immunostimulatory compound is expressed from the vector of the invention.

In a further embodiment, the 2A self-cleaving peptide is a 2A-peptide selected from the group consisting of T2A peptide, P2A peptide, E2A peptide and F2A peptide.

In one embodiment, the T2A peptide has an amino acid sequence identical to those T2A sequences listed in Table 1 or 2. In a further embodiment, the amino acid sequence DVEENPGP (SEQ ID NO: 50) is present but the remainder of the T2A amino acid sequence has 80% to 100% sequence identity to the T2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In another embodiment, the T2A peptide has the amino acid sequence with SEQ ID NO: 9.

In one embodiment, the P2A peptide has an amino acid sequence identical to those P2A sequences listed in Table 1 or 2. In a further embodiment, the sequence DVEENPGP (SEQ ID NO: 50) is present but the remainder of the P2A amino acid sequence has 80% to 100% sequence identity to the P2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In another embodiment, the P2A peptide has the amino acid sequence with SEQ ID NO: 11.

In one embodiment, the E2A peptide has an amino acid sequence identical to those E2A sequences listed in Table 1 or 2. In a further embodiment, the sequence DVESNPGP (SEQ ID NO: 173) is present but the remainder of the E2A amino acid sequence has 80% to 100% sequence identity to the E2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In another embodiment, the E2A peptide has the amino acid sequence with SEQ ID NO: 14.

In one embodiment, the F2A peptide has an amino acid sequence is identical to those F2A sequences listed in Table 1 or 2. In a further embodiment, the sequence DVESNPGP (SEQ ID NO: 173) is present but the remainder of the F2A amino acid sequence has 80% to 100% sequence identity to the F2A amino acid sequence of Table 1, such as 81%, 82%, 81%, 82%, 83%, 84%, 85%, 86%, 87%, 88%, 89%, 90%, 91%, 92%, 93%, 94%, 95%, 96%, 97%, 98% or 99% sequence identity. In another embodiment, the F2A peptide has the amino acid sequence with SEQ ID NO: 51.

TABLE 1

| Name | Sequence | SEQ ID NO. |
|---|---|---|
| T2A | EGRGSLLTCGDVEENPGP | SEQ ID NO: 9 |
| P2A | ATNFSLLKQAGDVEENPGP | SEQ ID NO: 11 |
| E2A | QCTNYALLKLAGDVESNPGP | SEQ ID NO: 14 |
| F2A | VKQTLNFDLLKLAGDVESNPGP | SEQ ID NO: 51 |

It is generally known that the efficiency of the 2A-peptides can be modulated to increase their efficiency in cleavage and expression, for example by inserting a GSG sequence prior to the N-terminus of the wild-type sequences, as shown in Table 2.

TABLE 2

| Name | Sequence | SEQ ID NO. |
|---|---|---|
| T2A | GSGEGRGSLLTCGDVEENPGP | SEQ ID NO: 52 |
| P2A | GSGATNFSLLKQAGDVEENPGP | SEQ ID NO: 53 |
| E2A | GSGQCTNYALLKLAGDVESNPGP | SEQ ID NO: 54 |
| F2A | GSGVKQTLNFDLLKLAGDVESNPGP | SEQ ID NO: 55 |

In another embodiment, the vector of the invention contains both IRES elements and nucleic acid sequences encoding 2A peptides. As an example, the vector comprises a first nucleic acid sequence encoding a first polypeptide, a second nucleic acid sequence encoding a first immunostimulatory compound and a third nucleic acid sequence encoding a second immunostimulatory compound. The vector may comprise an IRES element between the first and the second nucleic acid sequence and a nucleic acid sequence encoding a 2A peptide between the second and the third nucleic acid sequence. Alternatively, the vector may comprise a nucleic acid sequence encoding a 2A peptide between the first and the second nucleic acid sequence and an IRES element between the second and the third nucleic acid sequence. Further nucleic acid sequences encoding further immunostimulatory compounds may be included in the vector in the same manner.

In another embodiment, the vector of the invention contains nucleic acid sequences encoding two 2A peptides as a continuous sequence consisting of two 2A peptides. As an example, the vector comprises a first nucleic acid sequence encoding a first polypeptide and a second nucleic acid encoding an immunostimulatory compound. The vector may comprise a nucleic acid sequence encoding two 2A peptides as a continuous sequence between the first and the second nucleic acid sequence.

### Bidirectional Promoters

In one embodiment of the present disclosure, the vector comprises a co-expression element (or more than one co-expression element) which causes that the first polypeptide and the one or more immunostimulatory compounds are transcribed as separate transcripts, which results in separate transcription products and thus separate proteins.

In one embodiment of the present disclosure, the co-expression element is a bidirectional promoter, the concept of which is illustrated in FIG. 3a. Bidirectional promoters are typically short (e.g. <1 kbp) intergenic regions of DNA between the 5′ ends of the genes in a bidirectional gene pair. A “bidirectional gene pair” refers to two adjacent genes coded on opposite strands, with their 5′ ends oriented toward one another.

In one embodiment of the present disclosure, the bidirectional promoter is a back-to-back arrangement of CAG promoters with four CMV enhancers (Sladitschek H L, Neveu P A et al., PLOS One 11 (5), e0155177, 2016).

In one embodiment of the present disclosure, the bidirectional promoter is RPBSA (Kevin He et al., Int. J. Mol. Sci. 21 (23), 9256, 2020).

In one embodiment of the present disclosure, the bidirectional promoter is a back-to-back configuration of the mouse Pgk1 and human eukaryotic translation elongation factor 1 alpha 1 promoters (Golding & Mann, Gene Therapy 18, 817-826, 2011).

In one embodiment, the vector of the invention is a plasmid which comprises a first nucleic acid sequence encoding a first polypeptide and a second nucleic acid sequence encoding an immunostimulatory compound as a bidirectional gene pair comprising between their 5′ ends a bidirectional promoter.

### Multiple Promoters

In another embodiment of the present disclosure, the co-expression elements are various promoters, i.e. the vector is e.g. a plasmid which comprises a separate promoter for each of the nucleic acid sequences encoding the first polypeptide and the one or more immunostimulatory compounds, i.e. for separate transcription of the first polypeptide and each of the one or more immunostimulatory compounds.

In one embodiment, each of said nucleic acid sequence will have a different promoter, the concept of which is also illustrated in FIG. 3b. In one embodiment, all nucleic acid sequences have the same promoter to aim at equimolecular expression. In an alternative embodiment, one nucleic acid sequence has a stronger promoter than the other(s); that is, the nucleic acid sequence with a stronger promoter is likely to be expressed at higher levels than the other(s).

Numerous promoters are known in the art and suitable for inclusion into the plasmid of the invention. In one embodiment of the present disclosure, the promoter is derived from cytomegalovirus, such as the CMV promoter.

In one embodiment, the vector of the invention comprises one or more co-expression elements, preferably co-expression elements selected from the group consisting of IRES element, 2A peptide, bidirectional promoter and promoter.

The vector of the invention may comprise all kinds of combinations of co-expression elements.

As an example, the vector of the invention is a DNA plasmid which comprises a first nucleic acid sequence encoding a first polypeptide, a second nucleic acid sequence encoding a first immunostimulatory compound and a third nucleic acid sequence encoding a second immunostimulatory compound. In one embodiment, the DNA plasmid comprises an IRES and a 2A peptide which allows the co-expression of the first polypeptide (under control of a promoter) and of the first and second immunostimulatory compound. In another embodiment, the DNA plasmid comprises a bidirectional promoter and another promoter.

The skilled person will know that the terms first, second and third nucleic acid sequences as in the example above does not mean that the plasmid of the invention comprises the nucleic acid sequences in the order of first, second and third nucleic acid sequence. The second nucleic acid sequence may be downstream or upstream of the first or third nucleic acid sequence, the third nucleic acid sequence may be downstream or upstream of the first or second nucleic acid sequence and the first nucleic acid sequence may be upstream or downstream of the second or third nucleic acid sequence. In another embodiment, the first- and the second nucleic acid sequences might be in opposite directions on the same DNA strand, as may be the first and third or the second and third nucleic acid sequences. In further embodiments, the nucleic acid sequences encoding the first polypeptide and the immunostimulatory compounds might be on opposite DNA strands.

### Immunostimulatory Compounds

The vectors of the present invention comprise one or more nucleic acid sequences encoding one or more immunostimulatory compounds.

In one embodiment of the present disclosure, the immunostimulatory compound is a compound that affects antigen-presenting cells. In another embodiment, the immunostimulatory compound is a compound that stimulates antigen-presenting cells.

An antigen-presenting cell (APC) is a cell that displays antigens complexed with major histocompatibility complexes (MHCs) on their surfaces; this process is known as antigen presentation. T cells may recognize these complexes using their T-cell receptors (TCRs). APCs process antigens and present them to T-cells.

Almost all cell types can present antigens in some way. Professional APCs, including macrophages, such as Langerhans cells, B-cells and dendritic cells, present foreign antigens to helper T cells (CD4⁺) via MHC class II, while virus-infected cells (or cancer cells) can present antigens originating inside the cell to cytotoxic T cells (CD8⁺) via MHC class I. In addition to the MHC family of proteins, antigen presentation relies on other specialized signaling molecules on the surfaces of both APCs and T cells.

“MHC” stands for “major histocompatibility complex”. There are two primary classes of MHC molecules, MHC class I and MHC class II. The terms MHC class I and MHC class II are interchangeably used herein with HLA class I and HLA class II. HLA (human leukocyte antigen) is a major histocompatibility complex in humans.

APCs are vital for effective adaptive immune response, as the functioning of both cytotoxic and helper T cells is dependent on APCs. Antigen presentation allows for specificity of adaptive immunity and can contribute to immune responses against both intracellular and extracellular pathogens. It is also involved in defense against tumors.

In one embodiment of the present disclosure, the APCs are selected from the group consisting of dendritic cells, macrophages, Langerhans cells, B-cells and neutrophils and the immunostimulatory compound is a compound that affects such cells, such as stimulates such cells.

When APCs are affected, e.g. stimulated, the stimulation can result in attraction, activation, maturation and/or proliferation of the APCs.

In one embodiment of the present disclosure, the one or more immunostimulatory compounds promote attraction and/or activation and/or maturation and/or proliferation of antigen-presenting cells, e.g. promote the growth and/or expansion of antigen-presenting cells.

In one embodiment of the present disclosure, the immunostimulatory compound includes cytokines, chemokines, growth factors, ligands binding to members of the TNF receptor superfamily or ligands binding to pattern-recognition receptors (PRR).

In one embodiment, the vector of the invention is a plasmid, e.g. a DNA plasmid. It may be administered to a subject in need thereof, e.g. by intramuscular administration and the encoded compounds are expressed and secreted from the muscle cells. The efficacy of the first polypeptide (secreted in the form of a multimeric protein, such as a dimeric protein) may be enhanced by the co-expression of an immunostimulatory compound that attracts APCs to the injection site/muscle cells from which the multimeric protein and immunostimulatory compound are secreted. Attraction of APCs may result in a stronger and accelerated immune response: the multimeric protein is delivered to and taken up by APCs and not just diluted in the blood stream and there is a locally higher number of APCs which can present the one or more antigens comprised in the multimeric protein to other relevant immune cells.

Immunostimulatory Compounds that Promote the Attraction of APCs

Thus, in one embodiment, the immunostimulatory compound is one that promotes attraction of APCs.

APC attraction can be measured by methods known in the art, including in vitro trans-well assay or migration assays, by measuring surface markers in vivo of the muscle cells to which the vector of the invention is administered by flow cytometry or changes in the gene expression patterns by e.g. RT-qPCR, Nanostring or RNA sequencing.

One type of molecule capable of attracting APCs are chemokines. Chemokines are a family of small cytokines, or signaling proteins, secreted by cells. Their name is derived from their ability to induce directed chemotaxis in nearby responsive cells, i.e. they are chemotactic cytokines.

In one embodiment of the present disclosure, the immunostimulatory compound is a chemokine.

In another embodiment, the immunostimulatory compound can interact with the following surface molecules on APCs: CCR1 (C-C motif chemokine receptor 1), CCR3 (C-C motif chemokine receptor 3), CCR4 (C-C motif chemokine receptor 4), CCR5 (C-C motif chemokine receptor 5), CCR6 (C-C motif chemokine receptor 6), CCR 7 (C motif chemokine receptor 7), CCR8 (C-C motif chemokine receptor 8) or XCR1 (X-C motif chemokine receptor 1). In a preferred embodiment, the immunostimulatory compound can interact with the aforementioned surface molecules on human APCs.

In yet another embodiment of the present disclosure, the immunostimulatory compound is selected from the list consisting of macrophage inflammatory protein alpha and its isoforms, including mouse CCL3 (or MIP-1a), and human isoforms hCCL3, hCCL3L1, hCCL3L2 and hCCL3L3, preferably human MIP-1α (hMIP-1α variant, also called LD78B or CCL3L1), RANTES (CCL5), preferably human CCL5, such as human CCL5 having the amino acid sequence of SEQ ID NO: 43, chemokine ligand 4 (CCL4), preferably human CCL4, chemokine ligand 20 (CCL20), preferably human CCL20, chemokine ligand 19 (CCL19), preferably human CCL19, chemokine ligand 21 (CCL21), preferably human CCL21 and chemokine motif ligand 1 or 2 (XCL1 or XCL2), preferably human XCL1 or human XCL2.

The process of activation is the series of events that drives a resting APC towards a more differentiated and/or mature state. APCs are directly activated by interacting with pathogens/encountering a foreign antigen and indirectly by compounds (e.g. inflammatory mediators) produced and released by other cell types that recognize such molecules. APCs then undergo a series of cellular processes that culminate in their activation, which plays an important role of triggering effective immune responses to foreign antigens. For example, the maturation of dendritic cells is characterized by a reduction in phagocytic capacity, enhancement in antigen processing and presentation, improved migration to lymphoid tissues, and increase in the capacity to stimulate B- and T cells.

Immunostimulatory Compounds that Promote Activation and/or Maturation of APCs

In one embodiment of the present disclosure, the immunostimulatory compound is one that promotes activation and/or maturation of APCs.

Different techniques known in the art are available to measure the activation of APCs, e.g. comparing the cytokine profiles before and after activation as measured by ELISpot or FluoroSpot, determining overall changes in gene expression and analyzing expressed proteins (for example activation markers) by various techniques such as FACS, ELISA, WB and PCR/sequencing methods (qPCR (TaqMan arrays), Nanostring and RNA-seq).

In one embodiment of the present disclosure, the immunostimulatory compound can interact with a surface molecule on APCs which is selected from the group consisting of: a receptor of the TNF receptor superfamily, including CD40 (cluster of differentiation 40), CD137 (4-1BB), CD27, RANK and ICOS (CD278). In a preferred embodiment, the immunostimulatory compound can interact with the aforementioned surface molecules on human APCs.

Such an immunostimulatory compound may be selected from the list consisting of CD40L (CD40 ligand, CD154), CD137L (4-1BBL, 4-1BB ligand), CD70, ICOSL (CD275) and RANKL. In a preferred embodiment, the immunostimulatory compound is selected from the group consisting of hCD40L, hCD137L, hCD70, hICOSL and hRANKL

In another embodiment of the present disclosure, the immunostimulatory compound is a cytokine selected from the group consisting of IL-2, preferably human IL-2, IL-10, preferably human IL-10, IL-12, preferably human IL-12, such as human IL-12 comprising the amino acid sequences of SEQ ID NOs: 45 and 47, IL-21, preferably human IL-21 such as human IL-21 comprising the amino acid sequence of SEQ ID NO: 49, TNFα, preferably human TNFα, IFNγ, preferably human IFNγ and IL-1β, preferably human IL-1β.

In yet another embodiment of the present disclosure, the immunostimulatory compound is an immune signaling molecule such as MyD88 and TRIF, preferably such as human MyD88 and human TRIF, which activate APCs through TLR receptors present on their surfaces.

In yet another embodiment of the present disclosure, the immunostimulatory compound is a viral infection sensor such as for example RIG-1 and MDA-5, preferably human RIG-1 and human MDA-5.

In yet another embodiment of the present disclosure, the immunostimulatory compound is one that interacts with a pattern recognition receptor on APCs, e.g. a Toll-like receptor, including TLR2, TLR4 or TLR5. In a preferred embodiment, the immunostimulatory compound interacts with the aforementioned receptors on human APCs.

In one embodiment, such immunostimulatory compounds are selected from the list consisting of pathogen-associated molecular patterns (PAMPs), such as flagellin, protein damage-associated molecular patterns (DAMPs), such as HMGB1, heat-shock proteins (HSPs), Calrecticulin and Annexin A1. In a preferred embodiment, such immunostimulatory compounds are selected from the list consisting of human pathogen-associated molecular patterns (PAMPs), human protein damage-associated molecular patterns (DAMPs), such as human HMGB1, human heat-shock proteins (HSPs), human Calrecticulin and human Annexin A1. PAMPs/DAMPs include those which can be included as a nucleic acid sequence into the vector of the invention and will be expressed as functional proteins that may comprise functional groups introduced by post-translational modifications. The aforementioned molecules in turn activate the following receptors on APCs: RAGE, TLR4, TLR9 and TIM-3 (for HMGB1), FPR (for Annexin A1), SREC1, LOX1 and CD91 (for HSP). In a preferred embodiment, the immunostimulatory compound in turn activate the aforementioned receptors on human APCs.

Immunostimulatory Compounds that Promote Growth and/or Expansion of APCs

During an immune response, activated APCs undergo rapid expansion to fight infection or disease. Cell proliferation is the process by which a cell grows (increases in mass and size) and divides to produce two daughter cells. Growth factors stimulate cells by binding to receptors on the cell surface, which results in the proliferation of the cell. Cell proliferation leads to an exponential increase in cell number and is therefore a rapid mechanism of expanding the population of a cell. In the following, the terms “expansion” and “proliferation” are used interchangeably.

In one embodiment, the immunostimulatory compound is one that promotes growth and/or expansion of APCs.

Cell proliferation can be measured by different techniques known in the art for example by MTT/MTS assays, measuring protein translation or by labelling with CFSE. Well-known methods in the art are for example carried out by determining the metabolic activity of a cell population, which will reflect the condition of cell proliferation. Additionally, since the ATP content in cells is strictly controlled, the detection of ATP can also provide information on cell proliferation. Dead cells or imminent dead cells contain almost no ATP, and there is a strict linear relationship between the concentration of ATP measured in cell lysates or extracts and the number of cells. ATP detection using bioluminescent luciferase and its substrate, luciferin, can provide very sensitive results. If ATP is present, the luciferase will emit light, and the intensity of the luminescence is proportional to the ATP concentration. Furthermore, some antigens only exist in proliferating cells, while non-proliferating cells lack these antigens. Cell proliferation can be detected by utilizing specific monoclonal antibodies. For example, in human cells, the Ki-67 antibody recognizes the same-named protein which is expressed during all active phases of the cell cycle, but is absent in resting (quiescent) cells. Traditionally, radiolabeled 3H-thymine has been used as a measure of proliferation. It is incubated with cells for several hours or overnight. The newly proliferated cells will incorporate the radiolabels into their DNA, which can be detected by a scintillation counter after extraction.

In one embodiment of the present disclosure, the immunostimulatory compound can interact with the following surface molecules on APCs: GM-CSF-receptor (granulocyte-macrophage colony-stimulating factor receptor, CD116), FLT-3R (fms like tyrosine kinase 3, CD135), IL-15R or IL-4R. In a preferred embodiment, the immunostimulatory compound interacts with the aforementioned surface molecules on human APCs.

In one embodiment of the present disclosure, the immunostimulatory compound is a growth factor, such as GM-CSF (granulocyte-macrophage colony-stimulating factor), preferably human GM-CSF such as human GM-CSF having the amino acid sequence of SEQ ID NO: 41, FLT-3L (herein, the terms FLT-3L and FLT3L are used interchangeably), such as human FLT-3L, preferably human FLT-3L having the amino acid sequence of SEQ ID NO: 10, IL-15, preferably human IL-15 or IL-4, preferably human IL-14.

In another embodiment the immunostimulatory compound is one or more selected from Table 3 below. In a preferred embodiment, the immunostimulatory compounds listed in Table 3 are human immunostimulatory compounds which interact with the receptors listed in Table 3 present on human APCs:

TABLE 3

| Immunostimulatory | | |
|---|---|---|
| compound | Receptor | Effect on APC |
| IL-4 | IL-4R | Activation |
| IL-1β | IL-1R | Activation |
| IFNγ | IFNAR | Activation, maturation |
| IFNα | IFNAR | Activation, maturation |
| IL-15 | IL-15R | Activation |
| TNFα | TNFR1, TNFR2 | Maturation |
| IL-10 | IL-10R | Maturation |
| IL-12 | IL-12R | Maturation |
| IL-2 | IL-2R | Activation |
| MyD88 | TLRs | Activation, maturation |
| TRIF | TLRs | Activation, maturation |
| RIG-I | MAVS, VISA, IPS-1 | Activation, maturation |
| MDA-5 | MAVS, VISA, IPS-1 | Activation, maturation |
| P28 region of C3d | CR2 | Activation, maturation |
| IL-13 | IL-13R | Differentiation |
| IFNε | IFNAR | Activation, maturation |
| IFNκ | IFNAR | Activation, maturation |
| IFNβ | IFNAR | Activation, maturation |
| IFNβ | IFNAR | Activation, maturation |
| IL-6 | IL-6R | Differentiation |
| IL-21 | IL-21R | Activation, maturation |

In one embodiment of the present disclosure, the vector comprises nucleic acid sequences encoding 2, 3, 4, 5, 6, 7 or 8 immunostimulatory compounds. In another embodiment, the vector comprises nucleic acid sequences encoding 2 to 6 immunostimulatory compounds, i.e. 2 or 3 or 4 or 5 or 6 immunostimulatory compounds. The immunostimulatory compounds may be the same or different, preferably different.

In a preferred embodiment, the different immunostimulatory compounds also affect APCs differently, to stimulate the immune system on many different levels and by that maximize the therapeutic or prophylactic effect of the first polypeptide.

As an example, in one embodiment, the vector comprises nucleic acids encoding 3 different immunostimulatory compounds, with the first one being an immunostimulatory compound that promotes the attraction of DCs (e.g. XCL1), the second one being an immunostimulatory compound that promotes the growth of DCs (e.g. FLT3L) and the third one being an immunostimulatory compound that promotes activation of DCs (e.g. CD40L). In one embodiment, such a vector may be for use in the treatment and/or prevention of infectious diseases or the treatment of cancer. The selection of the particular immunostimulatory compounds will also depend on the targeting unit comprised in the first polypeptide, since said targeting unit targets APCs and may affect APCs in a similar manner as the immunostimulatory compound, e.g. attract or activate APCs.

### First Nucleic Acid Sequence

The vectors of the present disclosure comprise a first nucleic acid sequence, i.e. a DNA or RNA, including genomic DNA, cDNA and mRNA, either double-stranded or single-stranded, which encodes a first polypeptide. In one embodiment, the first nucleic acid sequence is a DNA. In another embodiment, the first nucleic acid sequence is optimized to the species of the subject to which it is administered. For administration to a human, in one embodiment, the first nucleic acid sequence is human codon optimized.

The first nucleic acid sequence encodes a first polypeptide, which comprises a targeting unit that targets antigen-presenting cells, a multimerization unit, such as a dimerization unit, and an antigenic unit comprising one or more antigens or parts thereof, e.g. one or more disease-relevant antigens or parts thereof. Once administered to a subject, the first polypeptide is expressed and, due to the presence of the multimerization unit, forms a multimeric protein, which elicits an immune response against the antigens or parts thereof, e.g. epitopes, comprised in the antigenic unit, resulting in the activation of the subject's immune system.

Structures like the first polypeptide and dimeric proteins or multimeric proteins comprising the first polypeptide are known in the art (e.g. WO 2004/076489A1, WO 2011/161244A1, WO 2017/118695A1 and WO 2022/013277A1, the disclosures of all are included herein by reference) and the skilled person can select a targeting unit that targets antigen-presenting cells, a multimerization unit, and an antigenic unit according to the envisaged use of the vector and the desired results following its administration.

The first polypeptide has an N-terminal start and a C-terminal end (illustrated in FIG. 4). The elements and units of the first polypeptide—targeting unit (TU), multimerization unit, such as, in this FIG. 4, a dimerization unit (DimU), and antigenic unit—may be arranged in the first polypeptide such that the antigenic unit is located at the C-terminal end of the first polypeptide (FIG. 4a) or at the N-terminal start of the first polypeptide (FIG. 4b). Preferably, the antigenic unit is located at the C-terminal end of the first polypeptide. A unit linker (UL) may connect the multimerization unit, such as a dimerization unit, and the antigenic unit. FIG. 4 illustrates an antigenic unit with 4 neoepitopes (neo1, neo2, neo3, neo4), which are separated by linkers (SUL1, SUL2, SUL3). An alternative way to describe the arrangement of the neoepitopes neo1-neo4 is that these neoepitopes are arranged in 3 antigenic subunits, each comprising a neoepitope and a subunit linker (SUL1, SUL2, SUL3), and a terminal neoepitope (neo4), which is closest to the C-terminal end or N-terminal start of the first polypeptide. The subunits are indicated in the Figure by square brackets. Thus, an antigenic unit comprising n neoepitopes comprises n−1 subunits, each subunit comprising a neoepitope and a subunit linker. As described herein, the 4 neoepitopes may be identical or different neoepitopes and the 3 linkers/subunit linkers may be identical or different. The order and orientation of the above-described units and elements of the first polypeptide is the same in the multimeric protein and in the first nucleic acid sequence encoding the first polypeptide. A first polypeptide as shown in FIG. 4 may be for use as an anticancer vaccine, e.g. personalized anticancer vaccine, as described herein.

In the following, the various units and elements of first polypeptide will be discussed in detail. They are present in the first nucleic acid sequence as nucleic acid sequences encoding the units/elements while they are present in the first polypeptide or multimeric protein as amino acids sequences. For the ease of reading, in the following, the units/elements are mainly explained in relation to the first polypeptide/multimeric protein, i.e. on the basis of their amino acid sequences.

### Targeting Unit

The first polypeptide encoded by the first nucleic acid comprised in the vectors of the invention comprises a targeting unit that targets APCs. APCs include dendritic cells (DCs) and subsets thereof.

The term “targeting unit” as used herein refers to a unit that delivers the polypeptide/multimeric protein to an antigen-presenting cell for MHC class II-restricted presentation to CD4+ T cells or for providing cross presentation to CD8+ T cells by MHC class I restriction.

Due to the presence of the targeting unit the multimeric protein attracts DCs, neutrophils and other immune cells. Thus, the multimeric protein will not only target the antigenic unit comprised therein to specific cells, but also facilitate a response-amplifying effect (adjuvant effect) by recruiting specific immune cells to the administration site of the vector.

The targeting unit is designed to target the multimeric protein to surface molecules expressed on the APCs, such as molecules expressed on any or many types of APCs or molecules exclusively on subsets of APCs, such as on subsets of DCs.

Examples of such surface molecules on APCs are HLA, cluster of differentiation 14 (CD14), cluster of differentiation 40 (CD40), CLEC9A, chemokine receptors and Toll-like receptors (TLRs). Chemokine receptors include C-C motif chemokine receptor 1 (CCR1), C-C motif chemokine receptor 3 (CCR3), C-C motif chemokine receptor 4 (CCR4), C-C motif chemokine receptor 5 (CCR5), C-C motif chemokine receptor 6 (CCR6), C-C motif chemokine receptor 7 (CCR7), C-C motif chemokine receptor 8 (CCR8) and XCR1. Toll-like receptors include TLR-2, TLR-4 and TLR-5. In one embodiment, the targeting unit is or comprises a moiety that interacts with these surface molecules. In a preferred embodiment, the aforementioned surface molecules are present on human APCs.

Thus, in one embodiment, the targeting unit comprises or consists of an antibody-binding region, such as the antibody variable domains (VL and VH), with specificity for MHC/HLA, CD14, CD40, CLEC9A or Toll-like receptors, preferably with specificity for. hCD14, hCD40, hCLEC9A or human Toll-like receptors. In another embodiment, the targeting unit comprises or consists of a synthetic or natural ligand. Examples include soluble CD40 ligand (CD40L), preferably hCD40L, natural ligands like chemokines, preferably such as in their human forms, e.g. chemokine ligand 5, also called C-C motif ligand 5 (CCL5 or RANTES), preferably hCCL5, such as hCCL5 with SEQ ID NO: 43, macrophage inflammatory protein alpha and its isoforms, including mouse CCL3 (or MIP-1a), and human isoforms hCCL3, hCCL3L1, hCCL3L2 and hCCL3L3, chemokine ligand 4 (CCL4) and its isoform CCL4L, preferably hCCL4 and hCCL4L, chemokine ligand 19 (CCL19), preferably hCCL19, chemokine ligand 20 (CCL20), preferably hCCL20, chemokine ligand 21 (CCL21), preferably hCCL21, chemokine motif ligand 1 or 2 (XCL1 or XCL2), preferably hXCL1 or hXCL2, and bacterial antigens like for example flagellin.

In one embodiment, the targeting unit has affinity for an MHC class II protein. Thus, in one embodiment, the targeting unit comprises or consists of an antibody-binding region, such as the antibody variable domains (VL and VH), with specificity for MHC class II proteins selected from the group consisting of anti-HLA-DP, anti-HLA-DR and anti-pan HLA class II.

In another embodiment, the targeting unit has affinity for a surface molecule selected from the group consisting of CD14, CD40, TLR-2, TLR-4 and TLR-5, preferably affinity for a surface molecule selected from the group consisting of hCD14, hCD40, hTLR-2, hTLR-4 and hTLR-5. Thus, in one embodiment the targeting unit comprises or consist of an antibody-binding region, such as the antibody variable domains (VL and VH), with specificity for CD14, CD40, TLR-2, TLR-4 or TLR-5, such as anti-CD14, anti-CD40, anti-TLR-2, anti-TLR-4 or anti-TLR-5, preferably with specificity for hCD14, hCD40, hTLR-2, hTLR-4 or hTLR-5, such as anti-hCD14, anti-hCD40, anti-hTLR-2, anti-hTLR-4 or anti-hTLR-5.

In yet another embodiment, the targeting unit comprises or consists of flagellin, which has affinity for TLR-5, such as hTLR-5. In yet another embodiment, the targeting unit comprises or consists of an antibody-binding region with specificity for CLEC9A, such as anti-CLEC9A or variants thereof, such as anti-CLEC9A Fv or the targeting unit comprises or consists of a CLEC9 ligand, e.g. a CLEC9 ligand comprising or consisting of the nucleic acid sequence with SEQ ID NO: 115 or an amino acid sequence encoded by said nucleic acid sequence. In a preferred embodiment, the targeting unit comprises or consists of an antibody-binding region with specificity for hCLEC9A, such as anti-hCLEC9A or variants thereof, such as anti-hCLEC9A Fv or the targeting unit comprises or consists of a human CLEC9 ligand.

Preferably, the targeting unit has affinity for a chemokine receptor selected from CCR1, CCR3, CCR5 and CCR7, more preferably for a chemokine receptor selected from CCR1, CCR3 and CCR5. In a further preferred embodiment, the targeting unit has affinity for a chemokine receptor selected from hCCR1, hCCR3, hCCR5 and hCCR7, more preferably for a chemokine receptor selected from hCCR1, hCCR3 and hCCR5.

In one embodiment, the targeting unit has affinity for the chemokine receptor CCR7, preferably for the human chemokine receptor CCR7. In another embodiment, the targeting unit comprises or consists of CCL19, such as CCL19 comprising or consisting of a nucleotide sequence of SEQ ID NO: 121 or an amino acid sequence encoded by said nucleotide sequence, or CCL21, such as the human forms of CCL19 or CCL21.

Preferably, the targeting comprises or consists of chemokine human macrophage inflammatory protein alpha (human MIP-1α (hMIP-1a) variant, also called LD78ß or CCL3L1), which binds to its cognate receptors, including CCR1, CCR3 and CCR5, expressed on the cell surface of APCs. The binding of the targeting unit to its cognate receptors leads to internalization of the multimeric protein into the APC and degradation of the protein into small peptides that are loaded onto MHC molecules and presented to CD4+ and CD8+ T cells to induce specific immune responses. Once stimulated, and with help from activated CD4+ T cells, CD8+ T cells will target and kill cells expressing the same antigens, e.g. cancer cells expression such same antigens.

In another embodiment, both a T cell response and a B cell response are induced. This also enables for an antibody response, i.e. antibodies binding to, for example, a viral surface protein when the virus is in circulation and neutralizing the virus by inhibiting it from entering the host cell.

In one preferred embodiment, the targeting unit comprises an amino acid sequence having at least 80% sequence identity to the amino acid sequence 24-93 of SEQ ID NO: 1, such as comprising the amino acid sequence 26-93 of SEQ ID NO: 1 or comprising the amino acid sequence 28-93 of SEQ ID NO: 1.

In a further preferred embodiment, the targeting unit comprises an amino acid sequence having at least 85% sequence identity to the amino acid sequence 24-93 of SEQ ID NO: 1, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence 24-93 of SEQ ID NO: 1.

In a more preferred embodiment, the targeting unit consists of an amino acid sequence having at least 80% sequence identity to the amino acid sequence 24-93 of SEQ ID NO: 1, such as consisting of the amino acid sequence 26-93 of SEQ ID NO: 1 or consisting of the amino acid sequence 28-93 of SEQ ID NO: 1.

In a further preferred embodiment, the targeting unit consists of an amino acid sequence having at least 85% sequence identity to the amino acid sequence 24-93 of SEQ ID NO: 1, such as at least 86% or at least 87%, 88%, 89%, 90%, 91%, 92%, 93%, 94%, 95%, 96%, 97%, 98% or at least 99% sequence identity. In yet another preferred embodiment, the targeting unit consists of the amino acid sequence 24-93 of SEQ ID NO: 1.

In one preferred embodiment, the targeting unit comprises the amino acid sequence 24-93 of SEQ ID NO: 1, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one comprising the amino acid sequence 26-93 of SEQ ID NO: 1 or one comprising the amino acid sequence 28-93 of SEQ ID NO: 1.

In another preferred embodiment, the targeting unit consists of the amino acid sequence 24-93 of SEQ ID NO: 1, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one consisting of the amino acid sequence 26-93 of SEQ ID NO: 1 or one consisting of the amino acid sequence 28-93 of SEQ ID NO: 1.

In one preferred embodiment, the targeting unit comprises a nucleic acid sequence having at least 80% sequence identity to the nucleic acid sequence with SEQ ID NO: 25.

In a further preferred embodiment, the targeting unit comprises a nucleic acid sequence having at least 85% sequence identity to the nucleic acid sequence with SEQ ID NO: 25, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the nucleic acid sequence of SEQ ID NO: 25.

In a more preferred embodiment, the targeting unit consists of a nucleic acid sequence having at least 80% sequence identity to the nucleic acid sequence with SEQ ID NO: 25.

In a further preferred embodiment, the targeting unit consists of a nucleic acid sequence having at least 85% sequence identity to the nucleic acid sequence of SEQ ID NO: 25, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleic acid sequence of SEQ ID NO: 25.

In one embodiment, a specific selection and/or combination of a target unit and immunostimulatory compounds in the vector of the invention is for example selecting hMIP-1α or CCL3 as targeting unit and selecting CCL4, GM-CSF, FLT3L and/or IFNα as immunostimulatory compounds. In another embodiment, a specific selection and/or combination is for example selecting hMIP-1α or CCL3 as targeting unit and selecting CCL5, GM-CSF, FLT3L and/or IFNα as immunostimulatory compounds. In yet another embodiment, a specific selection and/or combination is for example selecting CCL5 as targeting unit and selecting XCL1, GM-CSF, FLT3L and/or IFNα as immunostimulatory compounds. In yet another embodiment, a specific selection and/or combination is for example selecting hMIP-1α or CCL3 as targeting unit and selecting IL-4, GM-CSF, CD40L and/or TNFα as immunostimulatory compounds. In yet another embodiment, a specific selection and/or combination is for example selecting hMIP-1α or CCL3 as targeting unit and selecting IL-4, GM-CSF, IL-1B and/or TNFα as immunostimulatory compounds. In yet another embodiment, a specific selection and/or combination is for example selecting hMIP-1α or CCL3 as targeting unit and selecting IL-4, GM-CSF, IL-1ß and/or IFNγ as immunostimulatory compounds. In yet another embodiment, a specific selection and/or combination is for example selecting CCL5 as targeting unit and selecting CCL7, GM-CSF, FLT3L and/or IFNα as immunostimulatory compounds. In yet another embodiment, a specific selection and/or combination is for example selecting hMIP-1α or CCL3 as targeting unit and selecting 4-1BBL, GM-CSF, FLT3L and/or IFNα as immunostimulatory compounds. In yet another embodiment, a specific selection and/or combination is for example selecting hMIP-1α or CCL3 as targeting unit and selecting CD40L, GM-CSF, FLT3L and/or IFNα as immunostimulatory compounds. In yet another embodiment, a specific selection and/or combination is for example selecting hMIP-1α or CCL3 as targeting unit and selecting CD205, GM-CSF, FLT3L and/or IFNα as immunostimulatory compounds. In yet another embodiment, a specific selection and/or combination is for example selecting CCL5 as targeting unit and selecting 4-1BBL, GM-CSF, FLT3L and/or IFNα as immunostimulatory compounds. In yet another embodiment, a specific selection and/or combination is for example selecting CCL5 as targeting unit and selecting CD40L, GM-CSF, FLT3L and/or IFNα as immunostimulatory compounds. In yet another embodiment, a specific selection and/or combination is for example selecting anti-CD205 as targeting unit and selecting CCL5, GM-CSF, FLT3L and/or IFNα as immunostimulatory compounds. In yet another embodiment, a specific selection and/or combination is for example selecting hMIP-1α or CCL3 as targeting unit and selecting CCL4, GM-CSF, FLT3L and/or MyD88 as immunostimulatory compounds. In yet another embodiment, a specific selection and/or combination is for example selecting hMIP-1α or CCL3 as targeting unit and selecting TRIF, GM-CSF, FLT3L and/or MyD88 as immunostimulatory compounds. In yet another embodiment, a specific selection and/or combination is for example hMIP-1α or CCL3 as targeting unit and selecting GM-CSF, IL-12, IL-21 and/or CD40L as stimulatory compounds. In yet another embodiment, a specific selection and/or combination is for example selecting CD11c as targeting unit and selecting hMIP-1α or CCL3, IFNγ, GM-CSF and/or FLT3L as immunostimulatory compounds. In yet another embodiment, a specific selection and/or combination is for example selecting CD11c as targeting unit and selecting hMIP-1α or CCL3, TNFα, GM-CSF and/or FLT3L as immunostimulatory compounds. In yet another embodiment, a specific selection and/or combination is for example selecting CLEC9A as targeting unit and selecting CCL5, XCL1, GM-CSF and/or FLT3L as immunostimulatory compounds. In yet another embodiment, a selection and/or combination is for example selecting CD11c as targeting unit and selecting CCL5, XCL1, GM-CSF and/or FLT3L as immunostimulatory compounds. In yet another embodiment, a specific selection and/or combination is for example selecting CADM1 as targeting unit and selecting CCL5, XCL1, GM-CSF and/or FLT3L as immunostimulatory compounds. In yet another embodiment, a specific selection and/or combination is for example selecting CCL19 as a targeting unit and selecting GM-CSF, IL-12, IL-21 and/or CD40L as immunostimulatory compounds. In yet another embodiment, a specific selection and/or combination is for example selecting CCL19 as a targeting unit and selecting GM-CSF, CCL3L, XCL1 and/or CCL5 as immunostimulatory compounds.

In a preferred embodiment, the targeting units and immunostimulatory compounds listed in the previous paragraph are human proteins.

### Multimerization Unit/Dimerization Unit

The first polypeptide encoded by the first nucleic acid comprised in the vector of the invention comprises a multimerization unit, such as a dimerization unit.

The term “multimerization unit” as used herein refers to a sequence of nucleotides or amino acids between the antigenic unit and the targeting unit. In addition to connecting the antigenic unit and the targeting unit, the multimerization unit facilitates multimerization of/joins multiple polypeptides, such as two, three, four or more polypeptides, into a multimeric protein, such as a dimeric protein, a trimeric protein or a tetrameric protein. Furthermore, the multimerization unit also provides flexibility in the multimeric protein to allow optimal binding of the targeting unit to the surface molecules on the APCs, even if they are located at variable distances. The multimerization unit may be any unit that fulfils one or more of these requirements.

Multimerization Unit that Facilitates Multimerization of/Joins More than Two Polypeptides

In one embodiment, the multimerization unit is a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in one embodiment, the multimerization unit is a trimerization unit that comprises or consists of the nucleic acid sequence with SEQ ID NO: 116, or an amino acid sequence encoded by said nucleic acid sequence. In another embodiment, the trimerization unit is the C-terminal domain of T4 fibritin. Thus, in one embodiment, the multimerization unit is a trimerization unit that comprises or consists of the amino acid sequence with SEQ ID NO: 56.

In another embodiment, the multimerization unit is a tetramerization unit, such as a domain derived from p53, optionally further comprising a hinge region as described below. Thus, in one embodiment, the multimerization unit is a tetramerization unit that comprises or consists of the nucleic acid sequence with SEQ ID NO: 57, or an amino acid sequence encoded by said nucleic acid sequence, optionally further comprising a hinge region as described below.

### Dimerization Unit

The term “dimerization unit” as used herein, refers to a sequence of nucleotides or amino acids between the antigenic unit and the targeting unit. In addition to connecting the antigenic unit and the targeting unit, the dimerization unit facilitates dimerization of/joins two monomeric polypeptides into a dimeric protein. Furthermore, the dimerization unit also provides the flexibility in the dimeric protein to allow optimal binding of the targeting unit to the surface molecules on the APCs, even if they are located at variable distances. The dimerization unit may be any unit that fulfils these requirements.

Accordingly, in one embodiment the first polypeptide comprises a dimerization unit comprising a hinge region. In another embodiment, the dimerization unit comprises a hinge region and another domain that facilitates dimerization. In yet another embodiment, the dimerization unit comprises a hinge region, a dimerization unit linker and another domain that facilitates dimerization, wherein the dimerization unit linker connects the hinge region and the other domain that facilitates dimerization. In one embodiment, the dimerization unit linker is a glycine-serine rich linker, preferably GGGSSGGGSG (SEQ ID NO: 134), i.e. the dimerization unit comprises a glycine-serine rich dimerization unit linker and preferably the dimerization unit linker GGGSSGGGSG (SEQ ID NO: 134).

The term “hinge region” refers to an amino acid sequence comprised in the dimerization unit that contributes to joining two of the polypeptides, i.e. facilitates the formation of a dimeric protein. In the context of a multimerization unit that facilitates multimerization of/joins more than two polypeptides, the term “hinge region” refers to an amino acid sequence comprised in such multimerization unit that contributes to joining more than two polypeptides, e.g. three or four polypeptides and/or functioning as a flexible spacer, allowing the two targeting units of the multimeric protein to bind simultaneously to multiple surface molecules on APCs, even if they are located at variable distances.

Moreover, the hinge region functions as a flexible spacer, allowing the two targeting units of the dimeric protein to bind simultaneously to two surface molecules on APCs, even if they are located at variable distances. The hinge region may be Ig derived, such as derived from IgG, e.g. IgG1 or IgG2 or IgG3. In one embodiment, the hinge region is derived from IgM, e.g. comprising or consisting of the nucleotide sequence with SEQ ID NO: 119 or an amino acid sequence encoded by said nucleic acid sequence. The hinge region may contribute to the dimerization through the formation of covalent bond(s), e.g. disulfide bridge(s) between cysteines. Thus, in one embodiment, the hinge region has the ability to form one or more covalent bonds. Preferably, the covalent bond is a disulfide bridge.

In one embodiment, the dimerization unit comprises or consists of a hinge exon h1 and hinge exon h4 (human hinge region 1 and human hinge region 4), preferably hinge exon h1 and hinge exon h4 from IgG3, more preferably having an amino acid sequence of at least 80% sequence identity to the amino acid sequence 94-120 of SEQ ID NO: 1.

In a preferred embodiment, the dimerization unit comprises or consists of a hinge exon h1 and hinge exon h4 with an amino acid sequence of at least 85% sequence identity to the amino acid sequence 94-120 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a hinge exon h1 and hinge exon h4 with the amino acid sequence 94-120 of SEQ ID NO: 1.

In one preferred embodiment, the dimerization unit comprises or consists of the amino acid sequence 94-120 of SEQ ID NO: 1, except that at the most four amino acids have been substituted, deleted or inserted, such as at the most three amino acids, such as at the most two amino acids or such as at the most one amino acid.

In one preferred embodiment, the dimerization unit comprises or consists of a nucleic acid sequence having at least 80% sequence identity to the nucleic acid sequence with SEQ ID NO: 26.

In a further preferred embodiment, the dimerization unit comprises or consists of a nucleic acid sequence having at least 85% sequence identity to the nucleic acid sequence with SEQ ID NO: 26, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 26.

In another embodiment, the dimerization unit comprises another domain that facilitates dimerization, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG. More preferably, the other domain that facilitates dimerization is a carboxyterminal C domain derived from IgG3.

In one embodiment, the dimerization unit comprises or consists of a carboxyterminal C domain derived from IgG3 with an amino acid sequence having at least 80% sequence identity to the amino acid sequence 131-237 of SEQ ID NO: 1.

In a preferred embodiment, the dimerization unit comprises or consists of a carboxyterminal C domain derived from IgG3 with an amino acid sequence having at least 85% sequence identity to the amino acid sequence 131-237 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a carboxyterminal C domain derived from IgG3 with the amino acid sequence 131-237 of SEQ ID NO: 1.

In one preferred embodiment, the dimerization unit comprises or consists of the amino acid sequence 131-237 of SEQ ID NO: 1, except that at the most 16 amino acids have been substituted, deleted or inserted, such as at the most 15, 14, 13, 12, 11, 10, 9, 8, 7, 6, 5, 4, 3, 2, or 1 amino acid.

In one preferred embodiment, the dimerization unit comprises or consists of a nucleic acid sequence having at least 80% sequence identity to the nucleic acid sequence with SEQ ID NO: 27.

In a further preferred embodiment, the dimerization unit comprises or consists of a nucleic acid sequence having at least 85% sequence identity to the nucleic acid sequence with SEQ ID NO: 27, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises or consists of the nucleic acid sequence of SEQ ID NO: 27.

The immunoglobulin domain contributes to dimerization through non-covalent interactions, e.g. hydrophobic interactions. Thus, in one embodiment, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In a preferred embodiment, the dimerization unit comprises a hinge exon h1, a hinge exon h4, a dimerization unit linker and a CH3 domain of human IgG3. In a further preferred embodiment, the dimerization unit comprises a polypeptide consisting of hinge exon h1, hinge exon h4, a dimerization unit linker and a CH3 domain of human IgG3. In another preferred embodiment, the dimerization unit consists of a polypeptide consisting of hinge exon h1, hinge exon h4, a dimerization unit linker and a CH3 domain of human IgG3.

In one embodiment, the dimerization unit comprises an amino acid sequence having at least 80% sequence identity to the amino acid sequence 94-237 SEQ ID NO: 1.

In a preferred embodiment, the dimerization unit comprises an amino acid sequence having at least 85% sequence identity to the amino acid sequence 94-237 SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In an even more preferred embodiment, the dimerization unit comprises the amino acid sequence 94-237 of SEQ ID NO: 1.

In a more preferred embodiment the dimerization unit consists of an amino acid sequence having at least 80% sequence identity to the amino acid sequence 94-237 of SEQ ID NO: 1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence 94-237 of SEQ ID NO: 1.

In one preferred embodiment, the dimerization unit comprises or consists of the amino acid sequence 94-237 of SEQ ID NO: 1, except that at the most 28 amino acids have been substituted, deleted or inserted, such as at the most 25, 20, 15, 14, 13, 12, 11, 10, 9, 8, 7, 6, 5, 4, 3, 2 or 1 amino acids.

In one preferred embodiment, the dimerization unit comprises or consists of a nucleic acid sequence having at least 80% sequence identity to the nucleic acid sequence with SEQ ID NO: 28.

In a further preferred embodiment, the dimerization unit comprises or consists of a nucleic acid sequence having at least 85% sequence identity to the nucleic acid sequence with SEQ ID NO: 28, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 28.

In the first polypeptide encoded by the first nucleic acid sequence, the multimerization unit, e.g. dimerization unit, may have any orientation with respect to antigenic unit and targeting unit. In one embodiment, the antigenic unit is connected to the C-terminal end of the multimerization/dimerization unit (e.g. via a unit linker) with the targeting unit being connected to the N-terminal end of the multimerization/dimerization unit. In another embodiment, the antigenic unit is connected to the N-terminal end of the multimerization/dimerization unit (e.g. via a unit linker) with the targeting unit being connected to the C-terminal end of the multimerization/dimerization unit. It is preferred that the antigenic unit is connected to the C-terminal end of the multimerization/dimerization unit, e.g. via a linker, preferably via the unit linker, and the targeting unit is connected to the N-terminal end of the multimerization/dimerization unit.

### Antigenic Unit

Generally, the antigenic unit comprised in the first polypeptide/multimeric protein can comprise any type of antigen(s) or parts thereof, e.g. antigens or parts thereof which are disease-relevant. Examples include one or more cancer antigens or parts thereof or one or more antigens or parts thereof relevant for an infectious disease, i.e. a disease caused by a pathogen, including viruses, bacteria, fungi and parasites.

“Disease-relevant antigen(s)” or “antigen(s) which is/are relevant for a disease” is used herein to describe that the antigen(s) or parts thereof included in the antigenic unit play a role and have a relevance for a certain disease for which the vector of the invention comprising such antigenic unit is designed to be used. As an example, the antigenic unit comprises one or more cancer antigens or parts thereof and a vector comprising such antigenic unit is designed for use in the treatment of cancer. In another example, the antigenic unit comprises one or more infectious antigens or parts thereof, e.g. antigens derived from a pathogen and a vector comprising such antigenic unit is designed for use in the treatment of an infectious disease caused by such pathogen or wherein such pathogen is involved.

A “part” refers to a part/fragment of an antigen, i.e. part/fragment of the amino acid sequence of an antigen, or the nucleotide sequence encoding same, e.g. an epitope.

In one embodiment, the antigenic unit includes one T cell epitope. In another embodiment, the antigenic unit includes more than one T cell epitope, i.e. multiple T cell epitopes.

T cell epitopes suitable for inclusion into the antigenic unit may be known in the art, i.e. have been studied, proposed and/or verified to be involved and of relevance for a certain disease and published, e.g. in the scientific literature.

In one embodiment, the antigenic unit comprises T cell epitopes with a length of from 7 to 150 amino acids, preferably of from 7 to 100 amino acids, e.g. from 9 or 10 to 100 amino acids or from 15 to 100 amino acids or from 9 to 60 amino acids or from 9 to 30 amino acids or from 15 to 60 of from 15 to 30 or from 20 to 75 amino acids or from 25 to 50 amino acids.

In one embodiment, the antigenic unit comprised in the first polypeptide/multimeric protein comprises one or more antigens or parts thereof which are relevant for infectious diseases, e.g. antigens derived from pathogens.

Such antigens may be known or have been predicted in the art, i.e. have been studied, proposed and/or verified to be involved and of relevance for a certain infectious disease and published, e.g. in the scientific literature

In another embodiment, the antigenic unit comprised in the first polypeptide/multimeric protein comprises one or more antigens or parts thereof which are relevant for cancer, e.g. cancer antigens such as neoantigens or shared cancer antigens.

### Antigenic Unit of Individualized First Polypeptides

In one embodiment, the first polypeptide encoded by the first nucleic acid comprised in the vectors of the invention comprises an antigenic unit, which is designed specifically and only for the patient who is to be treated with such vector. Thus, the antigenic unit of such a first polypeptide comprises one or more patient-specific cancer antigens or parts thereof, such antigens including neoantigens or patient-present shared cancer antigens.

“Patient-present shared cancer antigen” is used herein to describe a shared cancer antigen or shared tumor antigen that has been identified to be present in the patient's tumor cells.

“Neoantigen” is used herein to describe a cancer antigen or tumor antigen found in a patient's tumor cells that comprises one or more mutations compared to the same patient's normal (i.e. healthy, non-cancerous) cells.

“Patient-present shared cancer epitope” is used herein to describe an amino acid sequence, or a nucleic acid sequence encoding same, comprised in a patient-present shared cancer antigen, which is known to be immunogenic or which has been predicted to be immunogenic.

“Neoepitope or patient-specific cancer epitope” is used herein to describe an amino acid sequence, or a nucleic acid sequence encoding same, comprised in a neoantigen or in a patient-specific cancer antigen, which comprises one or more mutations, which are predicted to be immunogenic.

Thus, in one embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more patient-specific cancer antigens or parts thereof, such as one or more patient-present shared cancer antigens or parts thereof and/or one or more neoantigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the antigenic unit comprises one or more patient-present shared cancer antigens or parts thereof, e.g. one patient-present shared cancer antigen or one or more parts of such patient-present shared cancer antigen, e.g. one or more epitopes, or several patient-present shared cancer antigens or one or more parts of such several patient-present shared cancer antigens, e.g. one or more epitopes.

The term “several” herein is used interchangeably with the term “multiple”, “a plurality” and “more than one”.

In another embodiment, the antigenic unit comprises one or more neoantigens or parts thereof, e.g. one neoantigen or one or more parts of such neoantigen, e.g. one or more neoepitopes or several neoantigens or one or more parts of such several neoantigens, e.g. one or more neoepitopes.

In yet another embodiment, the antigenic unit comprises any combinations of the aforementioned embodiments, i.e. any combination of one or more patient-present shared cancer antigens or parts thereof and of one or more neoantigens or parts thereof mentioned above.

### Antigenic Unit of Individualized Polypeptides Comprising One or More Neoantigens or Parts Thereof

Cancers develop from the patient's normal tissue by one or a few cells starting an abnormal, uncontrolled proliferation of the cells due to mutations. Although the cancer cells are mutated, most of the genome is intact and identical to the remaining cells in the patient. One approach of attacking a tumor is based on the knowledge that any tumor in any patient is unique: patient-specific mutations lead to expression of patient-specific mutated proteins, i.e. neoantigens that are unique for the particular patient. These neoantigens are not identical to any proteins in the normal cells of the patient.

Therefore, such neoantigens are suitable targets for a therapeutic pharmaceutical composition comprising vector of the invention which is manufactured specifically and only for the patient in question, i.e. an individualized anticancer vaccine.

The mutation may be any mutation leading to a change in at least one amino acid. Accordingly, the mutation may be one of the following:
a non-synonymous mutation leading to a change in the amino acid.
a mutation leading to a frame shift and thereby a completely different open reading frame in the direction after the mutation.
a read-through mutation in which a stop codon is modified or deleted leading to a longer protein with a tumor-specific epitope
splice mutations that lead to a unique tumor-specific protein sequence
chromosomal rearrangements that give rise to a chimeric protein with a tumor-specific epitope at the junction of the two proteins. When the mutation is due to a chromosomal rearrangement, the tumor-specific epitope can arise from a change in at least one amino acid or from a combination of two in-frame coding sequences.

In one embodiment, the antigenic unit comprises one or more neoantigens or parts thereof, such as one or more parts of one neoantigen or one or more parts of several neoantigens, preferably one or more neoepitopes and more preferably several neoepitopes. Such neoepitopes may be selected for inclusion into antigenic unit according to their predicted therapeutic efficacy, see WO 2017/118695A1, the disclosures of which is incorporated herein by reference.

Thus, in one embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more neoantigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the antigenic unit comprises one or more parts of one neoantigen or one or more parts of several neoantigens, preferably one or more neoepitopes. In a preferred embodiment, in the antigenic unit, the neoepitopes are separated by linkers. An alternative way to describe the separation of all neoepitopes by linkers is that all but the terminal neoepitope, i.e. the neoepitope at the N-terminal start of the first polypeptide or the C-terminal end of the first polypeptide, are arranged in antigenic subunits, wherein each subunit comprises a neoepitope and a subunit linker. Due to the separation of the neoepitopes by a linker, each neoepitope is presented in an optimal way to the immune system.

Hence, an antigenic unit that comprises n neoepitopes comprises n−1 antigenic subunits, wherein each subunit comprises a neoepitope and a subunit linker, and further comprises a terminal neoepitope. In one embodiment, n is an integer of from 1 to 50, e.g. 3 to 50 or 15 to 40 or 10 to 30 or 10 to 25 or 10 to 20 or 15 to 30 or 15 to 25 or 15 to 20. In a preferred embodiment, the antigenic subunit consists of a neoepitope and a subunit linker.

Thus, in a preferred embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises (i) n−1 antigenic subunits, each subunit comprising a neoepitope and a subunit linker, and (ii) a terminal neoepitope, and wherein n is the number of neoepitopes in said antigenic unit and n is an integer of from 1 to 50; and
(b) one or more further nucleic acid sequences encoding one or more immunostimulatory compounds,
wherein the vector allows for the co-expression of the first polypeptide and the one or more immunostimulatory compounds as separate molecules.

The neoepitope preferably has a length suitable for presentation by HLA molecules. Thus, in a preferred embodiment, the neoepitope has a length of from 7 to 30 amino acids. More preferred are neoepitopes having a length of from 7 to 10 amino acids or of from 13 to 30 amino acids, e.g. from 20 to 30 amino acids, e.g. 27 amino acids.

Preferably, the antigenic unit comprises a plurality of neoepitopes. In one embodiment, the antigenic unit comprises a plurality of different neoepitopes. In another embodiment, the antigenic unit comprises multiple copies of the same neoepitope. In yet another embodiment, the antigenic unit comprises several different neoepitopes and multiple copies of the same neoepitope.

Accordingly, a preferred approach is to include as many neoepitopes as possible in the antigenic unit (i.e. different and/or multiple copies of the same neoepitope) to thereby attack the cancer efficiently whilst not compromising the ability to activate T cells against the neoepitopes due to dilution of the desired T cell effect. Further, to secure that all neoepitopes are loaded efficiently to the same antigen-presenting cell, all neoepitope-encoding nucleotide sequences are comprised in a continuous polynucleotide chain resulting in the expression of a protein comprising all the neoepitopes instead of expressing each neoepitope as a discrete peptide.

To design the antigenic unit, the patient's tumor exome is analyzed to identify neoantigens. Preferably, the sequences of the most immunogenic neoepitopes from one or more neoantigens are selected for inclusion into the antigenic unit.

In one embodiment, the antigenic unit comprises at least 1 neoepitope. Preferably, the antigenic unit comprises at least 3 neoepitopes, more preferably at least 5 neoepitopes, such as 7 neoepitopes. In another more preferred embodiment, the antigenic unit comprises at least 10 neoepitope. In another more preferred embodiment, the antigenic unit comprises at least 15 neoepitopes, such as at least 20 or at least 25 or at least 30 or at least 35 or at least 40 or at least 45 neoepitopes.

Antigenic units comprising one or more neoepitopes are described in detail in WO 2017/118695A1. Any of such antigenic units can be used as antigenic unit in a first polypeptide encoded for in a vector of the invention for use in individualized anticancer therapy.

### Antigenic Unit of Individualized Polypeptides Comprising One or More Patient-Present Shared Cancer Antigens or Parts Thereof

Shared tumor antigens are expressed by many tumors, either across patients with the same cancer type, or across patients and cancer types. An example is the HPV16 antigen, a viral antigen that is expressed in about 50% of all patients with squamous cell carcinoma of the head and neck, but also in patients with other cancers such as cervical cancer and vulvar squamous cell carcinoma. Many of these shared antigens have previously been characterized as immunogenic and/or are known, i.e. their immunogenicity has been confirmed by appropriate methods and the results have been published, e.g. in a scientific publication. Others have already been predicted to be presented on specific HLA class I or class II alleles, e.g. by algorithms known in the art and their predicted immunogenicity has been published, e.g. in a scientific publication, without having confirmed their immunogenicity by appropriate methods.

In one embodiment, the antigenic unit comprises one or more patient-present shared cancer antigens or parts thereof, e.g. patient-present shared cancer epitopes, which are known to be immunogenic, have known expression patterns and/or are known or have already been predicted to bind to specific HLA class I and class II molecules.

T cells specific to patient-present shared cancer antigens can travel to the tumor and affect the tumor microenvironment, thus increasing the likelihood that additional tumor-specific T cells are able to attack the cancer.

Thus, in one embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more patient-present shared cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

Some patient-present shared cancer antigens are proteins comprising an amino acid sequence that comprise one or more mutations, i.e. patient-present shared cancer epitopes which are known to be immunogenic or which have been predicted to be immunogenic. Other patient-present shared cancer antigens are proteins which do not comprise mutations, e.g. overexpressed cellular proteins.

In one embodiment, the patient-present shared cancer antigen is selected from the group consisting of overexpressed cellular proteins, aberrantly expressed cellular proteins, cancer testis antigens, viral antigens, differentiation antigens, mutated oncogenes and mutated tumor suppressor genes, oncofetal antigens, shared fusion antigens, shared intron retention antigens, dark matter antigens and shared antigens caused by spliceosome mutations or frameshift mutations.

In one embodiment, the patient-present shared cancer antigen is an overexpressed or aberrantly expressed human cellular protein, i.e. a cellular protein found at increased levels in tumors compared with normal healthy cells and tissues. Examples of such overexpressed or aberrantly expressed cellular proteins include tumor protein D52, Her-2/neu, hTERT (telomerase) and survivin.

In another embodiment, the patient-present shared cancer antigen is a cancer testis antigen which is normally expressed in male germ cells in the testis but not in adult somatic tissues. In some cases, such antigens are also expressed in ovary and trophoblast. In malignancy, this gene regulation is disrupted, resulting in antigen expression in a proportion of tumors of various types. Examples of cancer testis antigens include MAGE-A, MAGE-B, GAGE, PAGE-1, SSX, HOM-MEL-40 (SSX2), NY-ESO-1, LAGE-1 and SCP-1.

In yet another embodiment, the patient-present shared cancer antigen is a differentiation antigen, for example tyrosinase.

In yet another embodiment, the patient-present shared antigen is a viral antigen. Examples of viral antigens include human papilloma virus (HPV), hepatitis B virus (HBV), Epstein-Barr virus (EBV), Kaposi's sarcoma-associated herpesvirus (KSHV), Merkel cell polyomavirus (MCV or MCPyV), human cytomegalovirus (HCMV) and human T-lymphotropic virus (HTLV).

In yet another embodiment, the patient-present shared cancer antigen is a mutated oncogene. Examples of mutated oncogenes include KRAS, CALR and TRP-2.

In yet another embodiment, the patient-present shared cancer antigen is a mutated tumor suppressor gene. Examples include mutated p53, mutated pRB, mutated BCL2 and mutated SWI/SNF.

In yet another embodiment, the patient-present shared cancer antigen is an oncofetal antigen, for example alpha-fetoprotein or carcinoembryonic antigen.

In yet another embodiment, the patient-present shared antigen is a shared intron retention antigen or shared antigen caused by frameshift mutation, for example CDX2 or CALR.

In yet another embodiment, the patient-present shared antigen is a shared antigen caused by spliceosome mutations. An example is an antigen caused by mutations like SF3B1 mut.

Generally, for any cancer antigen, immune tolerance has likely occurred when a patient presents with cancer. An anticancer vaccine should specifically trigger immune response to the antigens incorporated in the vaccine. In one embodiment, the first polypeptide encoded by the plasmid functions as an anticancer vaccine. The peripheral immune tolerance to the selected antigens may be weak or strong. By incorporating such patient-present shared cancer antigens or one or more parts thereof in the antigenic unit—either alone or together with other patient-present shared cancer antigens or parts thereof and/or neoantigens or neoepitopes—a polypeptide comprising such antigenic unit elicits an immune response which is strong and broad enough to affect the tumor microenvironment and change the patient's immune response against the tumor from a suppressive/tolerated type to a pro-inflammatory type. This may help to break tolerance to several other antigens, thus representing a considerable clinical benefit for the patient. The afore-described concept may be referred to as tipping the cancer immunity set point.

In one embodiment the antigenic unit comprises one or more patient-present shared cancer antigens or parts thereof that is a human cellular protein, preferably an overexpressed or aberrantly expressed human cellular protein or a differentiation antigen.

The patient-present shared cancer antigen can be detected in the tissue or body fluid of the patient by methods known in the art, including:
sequencing the patient's genome or exome and optionally searching, e.g. by tailor made software in whole genome/exome-seq data to e.g. identify mutated oncogenes or mutated tumor suppressor genes;
immunohistochemistry of the patient's tumor tissue, e.g. to detect the presence of mutated proteins;
RT-PCR, e.g. to detect the presence of viral antigens or known mutations in oncogenes;
ELISA using antibodies against e.g. mutated tumor proteins in serum samples;
RNA-seq of tumor tissue and comparison to healthy tissue to e.g. detect expression/over-expression of shared cancer antigens;
Searching, e.g. by tailor-made software in raw RNA sequence data to identify intron retention antigens;
searching, e.g. by tailor-made software, in whole genome-seq data to identify transposable elements which are elements of dark matter antigens;
detection of short repeats in raw whole exome/RNA sequence data to e.g. identify dark matter antigens;
RNA-seq data to e.g. identify shared viral antigens; and
comparing RNA-seq of the patient's tumor samples with either patient's own healthy tissue or a cohort/database (e.g. TCGA) versus consensus transcript expression, such as GTEX/HPA gene expression data.

In a preferred embodiment, the antigenic unit comprises one or more patient-present shared cancer antigens or part(s) of such antigen(s) that is known to be immunogenic, e.g. has previously been described to elicit an immune response in other patients, or has been predicted to bind to the patient's HLA class I and/or class II alleles.

In one embodiment, the antigenic unit comprises one or more patient-present shared cancer epitopes. In a preferred embodiment, such epitopes have a length suitable for presentation by the patient's HLA alleles.

In one embodiment, the antigenic unit comprises one or more patient-present shared cancer epitopes having a length suitable for specific presentation on HLA class I or HLA class II. In one embodiment, the epitope has a length of from 7 to 11 amino acids for HLA class I presentation. In another embodiment, the epitope has a length of from 13 to 30 amino acids for HLA class II presentation.

In one embodiment, the antigenic unit comprises one or more patient-present shared cancer epitopes having a length of from 7 to 30 amino acids, e.g. from 7 to 10 amino acids (such as 7, 8, 9, or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

The antigenic unit may comprise one or more patient-present shared cancer antigens either in full-length or one or more parts thereof.

In one embodiment, the antigenic unit comprises one patient-present shared cancer antigen in full-length. In another embodiment, the antigenic unit comprises several patient-present shared cancer antigens, each of them in full-length.

In yet another embodiment, the antigenic unit comprises one or more parts of a patient-present shared cancer antigen, e.g. one or more patient-present shared cancer epitopes. In yet another embodiment, the antigenic unit comprises one or more parts of several patient-present shared cancer antigens, e.g. one or more epitopes of several patient-present shared cancer antigens.

In yet another embodiment, the antigenic unit comprises one or more patient-present shared antigens in full-length and one or more parts of one or more patient-present shared cancer antigens. Examples include:
antigenic units comprising one patient-present shared antigen in full-length and one or more epitopes of one patient-present shared cancer antigen; and
antigenic units comprising several patient-present shared cancer antigens, each of them in full-length and one or more epitopes of one patient-present shared cancer antigen; and
antigenic units comprising one patient-present shared antigen in full-length and one or more epitopes of several patient-present shared cancer antigens; and
antigenic units comprising several patient-present shared cancer antigens, each of them in full-length and one or more epitopes of several patient-present shared cancer antigens.

In a preferred embodiment, the aforementioned epitopes are already known to be immunogenic, e.g. have been described to be immunogenic in the literature, or have already been predicted to bind to the patient's HLA class I and class II alleles, e.g. as described in the literature, preferably have already been predicted to bind to the patient's HLA class I alleles. In another preferred embodiment, the immunogenicity of the aforementioned epitopes is predicted, e.g. the binding of the epitopes to one or more of the patient's HLA class I and/or HLA class II molecules is predicted by methods known in the art, such as those disclosed in WO 2021/205027 A1, the disclosures of which is incorporated herein by reference, or those described herein, including those described in the section “Methods for designing an antigenic unit of an individualized first polypeptide”.

In one embodiment, the antigenic unit comprises 1 to 10 patient-present shared antigens in full-length.

In another embodiment, the antigenic unit comprises 1 to 30 parts of one or more patient-present shared antigens, wherein these parts include multiple epitopes that are predicted to bind to a patient's HLA class I or class II alleles. In yet another embodiment, the antigenic unit comprises 1 to 50 patient-present shared cancer epitopes, preferably epitopes that are predicted to bind to the patient's HLA class I or class II alleles.

### Antigenic Units of Individualized Polypeptides Comprising One or More Patient-Present Shared Cancer Antigens or Parts Thereof and One or More Neoantigens or Parts Thereof

In further embodiments, the antigenic units are a combination of all of the afore-described embodiments relating to antigenic units, which comprise one or more patient-present shared cancer antigens or parts thereof and all of the afore-described embodiments relating to antigenic units, which comprise one or more neoantigens or parts thereof.

Thus, in one embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit and an antigenic unit, wherein the antigenic unit comprises one or more patient-present shared cancer antigens or parts thereof and one or more neoantigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

Antigenic units comprising one or more patient-present shared cancer antigens or parts thereof and optionally one or more neoantigens and parts thereof are described in detail in WO 2021/205027A1, the content of which is included herein by reference. Any of such antigenic units can be used as antigenic unit in the first polypeptide encoded for in the vector of the invention for use in individualized anticancer therapy.

### Methods for Designing an Antigenic Unit of an Individualized First Polypeptide

The patient-present shared cancer antigens and neoantigens identified in a particular patient are preferably further processed to find those antigens that will render the first polypeptide most effective, when those antigens are included into the antigenic unit. The way and order in which such processing is done depends on how said antigens were identified, i.e. the data that form the basis for such processing.

In one embodiment, the processing and selecting of the antigen(s) to be included in the antigenic unit is carried out as follows:
1) A search in the literature and/or in one or more databases is carried out to retrieve information about and sequences of shared cancer antigens and preferably information about their expression pattern, immunogenicity or predicted immunogenicity, epitopes and HLA presentation. Such search is also carried out to determine whether the identified antigen is a patient-present shared cancer antigen or a neoantigen.
2) If it was determined that the identified antigen is a patient-present shared cancer antigen, the sequence thereof is studied to identify epitopes, preferably all epitopes, that are predicted to bind to the patient's HLA class I/II alleles. The prediction may be carried out by using prediction tools known in the art, e.g. prediction software known in the art, such as NetMHCpan and similar software.
3) The most promising sequences of the patient-present shared cancer antigen which are most immunogenic or predicted to be most immunogenic, i.e. those that show predicted binding to one or more of the patient's HLA class I/II alleles, are selected for inclusion into the antigenic unit. In one embodiment, minimal epitopes are selected, e.g. if only a few promising epitopes were identified in step 2 or if longer stretches of non-immunogenic sequences are present between the epitopes. In another embodiment, a longer sequence is selected which comprises several epitopes that bind to the patient's specific HLA alleles. In yet another embodiment, the full-length sequence of the antigen is selected for inclusion into the antigenic unit.
4) The most promising parts of neoantigen sequences, e.g. neoepitopes, are selected for inclusion into the antigenic unit based on predicted immunogenicity and binding to the patient's HLA class I/II alleles of such sequences.

Tumor mutations are discovered by sequencing of tumor and normal tissue and comparing the obtained sequences from the tumor tissue to those of the normal tissue. A variety of methods is available for detecting the presence of a particular mutation or allele in a patient's DNA or RNA. Such methods include dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. Alternatively, mutations may be identified by direct protein sequencing.

Out of the maybe hundreds or thousands of mutations in the tumor exome, the most promising sequences are selected in silico based on predictive HLA-binding algorithms. The intention is to identify all relevant epitopes and after a ranking or scoring, determine the sequences to be included in the antigenic unit. Methods known in the art may suitable for scoring, ranking and selecting neoepitopes include those disclosed in WO 2020/065023A1 and WO 2020/221/783A1.

Further, any suitable algorithm for such scoring and ranking may be used, including the following:

Available free software analysis of peptide-MHC binding (IEDB and NetMHCpan) that can be downloaded from the following websites:
www.iedb.org/
www.cbs.dtu.dk/services/NetMHC/

Commercially available advanced software to predict optimal sequences for vaccine design are found here:
www.oncoimmunity.com/
omictools.com/t-cell-epitopes-category
github.com/griffithlab/pVAC-Seq
crdd.osdd.net/raghava/cancertope/help.php
www.epivax.com/tag/neoantigen/

Each mutation is scored with respect to its antigenicity, and the most antigenic neoepitopes are selected and optimally arranged in the antigenic unit.

### Antigenic Unit of Non-Individualized First Polypeptides

### Antigenic Units of First Polypeptides Comprising One or More Shared Cancer Antigens or Parts Thereof

A non-individualized or “off-the-self” vector encoding a first polypeptide (also referred to as first polypeptide comprising shared cancer antigen(s)) comprises a polynucleotide sequence encoding an antigenic unit, which comprises one or more shared cancer antigens or parts thereof.

“Shared cancer antigen” or “shared tumor antigen” is used herein to describe an antigen that has been described to be expressed by many tumors, either across patients with the same cancer type, or across patients and cancer types.

“Shared cancer epitope” is used herein to describe an amino acid sequence comprised in a shared cancer antigen, which is known or predicted to be immunogenic.

In one embodiment, the antigenic unit non-individualized first polypeptides for use in the treatment of cancer comprises one or more shared cancer antigens or parts thereof, e.g. shared cancer epitopes, which are known to be immunogenic, have known expression patterns and/or are known or have already been predicted to bind to specific HLA class I and class II molecules.

Thus, in one embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more shared cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

Some shared cancer antigens are proteins comprising an amino acid sequence that comprise one or more mutations, i.e. shared cancer epitopes which are known to be immunogenic or which have been predicted to be immunogenic. Other shared cancer antigens are proteins which do not comprise mutations, e.g. overexpressed cellular proteins.

In one embodiment, the shared cancer antigen is selected from the group consisting of overexpressed cellular proteins, aberrantly expressed cellular proteins, cancer testis antigens, viral antigens, differentiation antigens, mutated oncogenes and mutated tumor suppressor genes, oncofetal antigens, shared fusion antigens, shared intron retention antigens, dark matter antigens and shared antigens caused by spliceosome mutations or frameshift mutations.

In one embodiment, the shared cancer antigen is an overexpressed or aberrantly expressed human cellular protein, i.e. a cellular protein found at increased levels in tumors compared with normal healthy cells and tissues. Examples of such overexpressed or aberrantly expressed cellular proteins include tumor protein D52, Her-2/neu, hTERT (telomerase) and survivin.

In another embodiment, the shared cancer antigen is a cancer testis antigen which is normally expressed in male germ cells in the testis but not in adult somatic tissues. In some cases, such antigens are also expressed in ovary and trophoblast. In malignancy, this gene regulation is disrupted, resulting in antigen expression in a proportion of tumors of various types. Examples of cancer testis antigens include MAGE-A, MAGE-B, GAGE, PAGE-1, SSX, HOM-MEL-40 (SSX2), NY-ESO-1, LAGE-1 and SCP-1.

In yet another embodiment, the shared cancer antigen is a differentiation antigen, for example tyrosinase.

In yet another embodiment, the shared antigen is a viral antigen. Examples of viral antigens include human papilloma virus (HPV), hepatitis B virus (HBV), Epstein-Barr virus (EBV), Kaposi's sarcoma-associated herpesvirus (KSHV), Merkel cell polyomavirus (MCV or MCPyV), human cytomegalovirus (HCMV) and human T-lymphotropic virus (HTLV).

In yet another embodiment, the shared cancer antigen is a mutated oncogene. Examples of mutated oncogenes include KRAS, CALR and TRP-2.

In yet another embodiment, the shared cancer antigen is a mutated tumor suppressor gene. Examples include mutated p53, mutated pRB, mutated BCL2 and mutated SWI/SNF.

In yet another embodiment, the shared cancer antigen is an oncofetal antigen, for example alpha-fetoprotein or carcinoembryonic antigen.

In yet another embodiment, the shared antigen is a shared intron retention antigen or shared antigen caused by frameshift mutation, for example CDX2 or CALR.

In yet another embodiment, the shared antigen is a shared antigen caused by spliceosome mutations. An example is an antigen caused by mutations like SF3B1 mut.

Further examples of shared cancer antigens include scFvs derived from a monoclonal Ig produced by myeloma or lymphoma, also called the myeloma/lymphoma M component in patients with B cell lymphoma or multiple myeloma, HIV derived sequences like e. g. gpl20 or Gag derived sequences, tyrosinase related protein (TRP)-1, melanoma antigen, prostate specific antigen and idiotypes, HPV antigens selected from the list consisting of E1, E2, E6, E7, L1 and L2, e.g. E6 and/or E7 of HPV16 and/or HPV18.

Any shared cancer antigen sequence of sufficient length that includes a specific epitope may be used as the antigenic unit. Accordingly, in one embodiment, the antigenic unit comprises an amino acid sequence of at least 7 amino acids, such as at least 8 amino acids, corresponding to at least about 21 nucleotides, such as at least 24 nucleotides, in a nucleic acid sequence encoding such antigenic unit.

In yet another embodiment, the antigenic unit comprises one or more parts of a shared cancer antigen, e.g. one or more shared cancer epitopes. In yet another embodiment, the antigenic unit comprises one or more parts of several shared cancer antigens, e.g. one or more epitopes of several shared cancer antigens. In yet another embodiment, the antigenic unit comprises one or more shared antigens in full-length and one or more parts of one or more shared cancer antigens. Examples include:
antigenic units comprising one shared antigen in full-length and one or more epitopes of one shared cancer antigen; and
antigenic units comprising several shared cancer antigens, each of them in full-length and one or more epitopes of one shared cancer antigen; and
antigenic units comprising one shared antigen in full-length and one or more epitopes of several shared cancer antigens; and
antigenic units comprising several shared cancer antigens, each of them in full-length and one or more epitopes of several shared cancer antigens.

Examples of polypeptides comprising shared antigens against HPV are disclosed in WO 2013/092875A1, the content of which is incorporated herein by reference.

### Methods for Designing an Antigenic Unit of a First Polypeptide Comprising Shared Cancer Antigen(s)

Also, for vectors encoding first polypeptides comprising shared cancer antigen(s), the antigenic unit is designed to include those sequences that are likely to render the polypeptide effective in a variety of patients, e.g. patients having a certain type of cancer.

In one embodiment, the selection of the antigen to be included in the antigenic unit is carried out by performing a search in the literature and/or in one or more databases to retrieve information about and sequences of shared cancer antigens and preferably information about their expression pattern, immunogenicity or predicted immunogenicity, epitopes and/or HLA presentation. Epitopes are then identified that are known or predicted to bind to a variety of HLA class I/II alleles of many patients or that bind a certain subset of HLA class I/II alleles which is dominant in a certain cancer indication and/or a certain patient population across different cancer indications. Preferably, the most promising, i.e. the sequences of the shared cancer antigen which are most immunogenic or predicted to be most immunogenic, are selected for inclusion into the antigenic unit.

### Antigenic Units of First Polypeptides Comprising One or More Infectious Antigens or Parts Thereof

In another aspect of the invention, the first polypeptide encoded by the first nucleic acid comprised in the vectors of the invention comprises an antigenic unit, which is designed for the treatment of an infectious disease and the vector/first polypeptide is for use in the treatment of an infectious disease.

In one embodiment, the antigenic unit comprised in the first polypeptide comprises one or more antigens or parts thereof which are relevant for infectious diseases, i.e. one or more infectious antigens, i.e. antigens or parts thereof derived from pathogens.

“Infectious disease” is used herein to describe a condition caused by a pathogen or a condition wherein a pathogen is involved in causing it. An example of the latter are eggs of a parasite, which do not cause the disease itself but develop into larvae which cause it.

“A pathogen” includes viruses, bacteria, fungi and parasites.

The antigens described in this section are “infectious antigens”, i.e. antigens derived from pathogens, i.e. they are comprised (or naturally found) in proteins of a pathogen which causes the disease or is involved in causing it. The terms “infectious antigen” and “antigen derived from a pathogen” may be used herein interchangeably.

Thus, in one embodiment, the invention relates to a vector comprising:
(a) a first nucleic acid sequence encoding a first polypeptide, wherein the first polypeptide comprises a targeting unit a targeting unit that targets antigen-presenting cells, a multimerization unit, such as a dimerization unit, and an antigenic unit, wherein the antigenic unit comprises one or more infectious antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

In another embodiment, the invention relates to a vector comprising:
(a) a first nucleic acid sequence encoding a first polypeptide, wherein the first polypeptide comprises a targeting unit a targeting unit that targets antigen-presenting cells, a multimerization unit, such as a dimerization unit, and an antigenic unit, wherein the antigenic unit comprises one or more antigens derived from one or more pathogens or parts of such antigens; and
(b) one or more further nucleic acid sequences encoding one or more immunostimulatory compounds,
wherein the vector allows for the co-expression of the first polypeptide and the one or more immunostimulatory compounds as separate molecules.

In the above-described embodiment, the antigenic unit comprises one or more antigens derived from a pathogen or parts of such antigens, e.g. one antigen derived from a pathogen or more than one antigen derived from a pathogen, i.e. multiple antigens derived from a pathogen, e.g. comprised in the same or different proteins of such pathogen.

In one embodiment, the antigenic unit comprises one or more antigens derived from multiple pathogens or parts of such antigens. In one embodiment, the multiple pathogens are multiple different pathogens. In that context, a “different pathogen” may, for example, be a different virus or bacterium or a different strain of the same virus or bacterium or it may be the same strain, but comprising one or more mutations.

A vector comprising one or more antigens or parts thereof derived from multiple pathogens may be for use in a pan-vaccine, e.g. a vaccine targeting different (seasonal) viruses. For example, the pan-vaccine could target betacoronavirus and influenza or target different strains of e.g. betacoronaviruses or different mutations of the same strain.

Examples of infectious antigens/antigens that are derived from pathogens are such of bacterial origin, e.g. tuberculosis antigens and OMP31 from brucellosis, or viral origin, e.g. HIV derived sequences like e.g. gp120 derived sequences, glycoprotein D from HSV-2, and influenza virus antigens like hemagglutinin, nucleoprotein and M2, and HPV derived antigens such as E1, E2, E6, E7, L1 or L2, such as E6 and E7 of HPV16 or HPV18.

In one embodiment, the antigenic unit comprises one or more betacoronavirus antigens or parts thereof.

Betacoronaviruses denotes a genus in the subfamily Orthocoronaviridae. Betacoronaviruses are enveloped, positive-sense single-stranded RNA viruses. Within the genus, four lineages are commonly recognized: lineage A (subgenus Embecovirus), lineage B (subgenus Sarbecovirus), lineage C (Merbecovirus) and lineage D (Nobecovirus). Betacoronaviruses include the following viruses which caused/cause epidemics/pandemics in humans or can infect humans: SARS-CoV, which causes severe acute respiratory syndrome (SARS), MERS-CoV, which causes Middle East respiratory syndrome (MERS), SARS-CoV-2, which causes coronavirus disease 2019 (Covid-19), HCoV-OC43 and HCoV-HKU1. SARS-CoV and SARS-CoV-2 belong to the lineage B (subgenus Sarbecovirus), MERS-CoV belongs to the lineage C (Merbecovirus) and HCoV-OC43 and HCoV-HKU1 belong to the lineage A (subgenus Embecovirus).

In one embodiment, the antigen is the spike protein of SARS-CoV or SARS-CoV-2, or a part thereof.

In one embodiment of the present invention, the antigen may be a T cell epitope which is a part of the sequence of the spike protein or the membrane protein or the envelope protein or the nucleocapsid protein or the ORF1a/b or ORF3a protein. In another embodiment, the T cell epitope is part of the following genes/proteins: NCAP, AP3A, spike, ORF1a/b, ORF3a, VME1 and VEMP.

In some embodiments, the antigenic unit of the vector of the invention comprises one or more antigens or parts thereof derived from one or more pathogens selected from the list consisting of influenza virus, Herpes simplex virus, CMV, HPV, HBV, brucella bacteria, HIV, HSV-2 and Mycobacterium tuberculosis bacteria.

The vector of the invention for use in the treatment of infectious diseases is ideal for fighting pandemics and epidemics as it can induce rapid, strong immune response. Such a vector is designed to induce an antigenic effect through inclusion into the antigenic unit of the full-length or a part of one or more infectious antigens, such parts may for example be selected T cell epitopes, or through combinations thereof. In one embodiment, the targeting unit of such a first polypeptide is anti-pan-HLA class II or human MIP-1α, and an immune response will be raised through B cells and/or T cells. In one embodiment, the vector can be used in a prophylactic setting or a therapeutic setting or both a prophylactic and a therapeutic setting.

Antigenic Units of First Polypeptides Comprising One or More T Cell Epitopes from One or More Pathogens

In one embodiment, the antigenic unit of a vector/first polypeptide for use in the treatment of an infectious disease comprises at least one T cell epitope from one or more pathogens. Such T cell epitopes are comprised (or naturally found) in proteins of pathogens. Conserved parts of the genome among many pathogens comprise T cell epitopes capable of initiating immune responses.

Thus, one aspect of the invention relates to a vector comprising:
(a) a first nucleic acid sequence encoding a first polypeptide, wherein the first polypeptide comprises a targeting unit a targeting unit that targets antigen-presenting cells, a multimerization unit, such as a dimerization unit, and an antigenic unit, wherein the antigenic unit comprises at least one T cell epitope from one or more infectious antigens; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the invention relates to a vector comprising:
(a) a first nucleic acid sequence encoding a first polypeptide, wherein the first polypeptide comprises a targeting unit a targeting unit that targets antigen-presenting cells, a multimerization unit, such as a dimerization unit, and an antigenic unit, wherein the antigenic unit comprises at least one T cell epitope derived from one or more pathogens; and
(b) one or more further nucleic acid sequences encoding one or more immunostimulatory compounds,
wherein the vector allows for the co-expression of the first polypeptide and the one or more immunostimulatory compounds as separate molecules.

In some embodiments, the antigenic unit comprises at least one T cell epitope of a pathogen, i.e. one T cell epitope of a pathogen or more than one T cell epitope of a pathogen, i.e. multiple T cell epitopes of a pathogen. In some embodiments, the multiple T cell epitopes are of the same pathogen, i.e. (naturally) comprised in the same or different proteins of the pathogen. In other embodiments, the multiple T cell epitopes are of multiple different pathogens, i.e. (naturally) comprised in protein of different pathogens.

The at least one T cell epitope comprised in the antigenic unit has a length of from 7 to about 200 amino acids, with a longer T cell epitope possibly including hotspots of minimal T cell epitopes. A “hotspot of minimal epitopes is a region that contains several minimal T cell epitopes (e.g. having a length of from 7-15 amino acids) that are predicted to be presented by different HLA alleles to cover a broad range of world population.

In some embodiments, the antigenic unit comprises at least one T cell epitope with a length of from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids.

A T cell epitope having a length of about 60 to 200 amino acids may be split into shorter sequences and included into the antigenic unit separated by linkers, e.g. linkers as described herein. By way of example, a T cell epitope having a length of 150 amino acids may be split into 3 sequences of 50 amino acids each, and included into the antigenic unit, with linkers separating the 3 sequences from each other.

In one embodiment, the antigenic unit comprises multiple T cell epitopes which are separated from each other by linkers, e.g. linkers as discussed herein, e.g. linkers as discussed in the “linkers in the antigenic unit” section herein.

In some embodiments, the at least one T cell epitope has a length suitable for presentation by MHC. Thus, in some embodiments, the antigenic unit comprises at least one T cell epitopes having a length suitable for specific presentation on MHC class I or MHC class II. In some embodiments, the at least one T cell epitope has a length of from 7 to 11 amino acids for MHC class I presentation. In other embodiments, the at least one T cell epitope has a length of about 15 amino acids for MHC class II presentation.

The number of T cell epitopes in the antigenic unit may vary, and depends on the length and number of other elements included in the antigenic unit, e.g. linkers.

In some embodiments, the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.

In a preferred embodiment, the at least one T cell epitope is from a conserved region of the pathogen, i.e. is conserved between several subgenera, species or strains of a respective pathogen.

The T cell epitopes may be comprised in any of the pathogen's proteins, i.e. in surface proteins but also in the internal proteins such as viral nucleocapsid proteins or viral replicase polyproteins or in other structural and non-structural proteins.

A vector comprising an antigenic unit comprising T cell epitopes from conserved regions of pathogens will provide protection against several species/strains of the pathogen. Such a vector will also provide protection against multiple variants of a pathogen, which is important for the efficacy of such a vector/first polypeptide against future mutated pathogens. Viruses are known to mutate, e.g. undergo viral antigen drift or antigen shift. The finding of conserved regions across a viral genus makes it likely that these conserved regions are needed to maintain essential structures or functions, thus it is anticipated that future mutations will take place in the less-conserved regions. By raising an immune response against the conserved regions, the individual treated with plasmid will be protected also against mutated (and thus novel) strains of the future.

In one embodiment of the present invention, the antigenic unit is therefore designed to evoke a cell-mediated immune response through activation of T cells against the T cell epitopes of the infectious antigen/from a pathogen included in such antigenic unit. T cells recognize epitopes when they have been processed and presented complexed to an MHC molecule.

In one embodiment, the T cell epitope is known in the art, e.g. one that has been studied and described in the literature, e.g. known to be immunogenic, e.g. its immunogenicity has been confirmed by appropriate methods and the results have been published, e.g. in a scientific publication. In one embodiment, the antigenic unit includes multiple T cell epitopes that are known to be immunogenic.

For example, useful T cell epitopes known in the art are those against infection by SARS-CoV2 in humans can be found in Grifoni et al., Cell Host Microbe. 2021 Jul. 14; 29(7): 1076-1092. Such T cell epitopes may thus be included in the antigenic unit of vectors for use in treating SARS-CoV2 in humans. Another example of such T cell epitopes is the T cell epitope with the sequence CTELKLSDY (SEQ ID NO: 82) of the nucleoprotein from influenza A virus, the T cell epitope with the sequence NLVPMVATV (SEQ ID NO: 83) of the 65 kDa phosphoprotein from human herpesvirus 5 (human cytomegalovirus) and the T cell epitope with the sequence KLVANNTRL (SEQ ID NO: 84) of diacylglycerol acyltransferase/mycolyltransferase Ag85B from Mycobacterium tuberculosis.

As an example, the at least one T cell epitope may be from a region of a human papilloma virus (HPV), e.g. from HPV16 or HPV18, e.g. at least one T cell epitope comprised in HPV antigens from the group consisting of E1, E2, E6, E7, L1 and L2, e.g. E6 and/or E7 of HPV16 and/or HPV18. By including such T cell epitopes in the vectors of the disclosure, a pharmaceutical composition comprising such vector may will provide protection against HPV. HPV infections are involved in certain cancers, such as squamous cell carcinoma of the head and neck, cervical cancer and vulvar squamous cell carcinoma. Indeed, HPV16 viral antigens are expressed in about 50% of all patients with said cancers.

As another example, the at least one T cell epitope may be from a region of a human influenza virus, such as human influenza virus A, human influenza virus B, human influenza virus C and human influenza virus D. As an example, the human influenza virus may be a specific hemagglutinin (HA) subtype, such as H1, H2, and H3, and/or a specific neuraminidase (NA) subtype, such as N1 or N5. As an example, the human influenza virus may be a H1N1 subtype. Such T cell epitopes may thus be included in the antigenic unit of a vector of the disclosure for use in the treatment of influenza infections.

In another embodiment, the T cell epitope is predicted to be immunogenic, e.g. is selected based on the predicted ability to bind to HLA class I/II alleles. In one embodiment, the antigenic unit includes multiple T cell epitopes, e.g. multiple T cell epitopes that are separated from each other by linkers, e.g. linkers as discussed herein, e.g. as discussed in the “linkers in the antigenic unit” section herein, that are predicted to bind to HLA class I/II alleles. The T cell epitopes are selected in silico on the basis of predictive HLA-binding algorithms. After having identified all relevant epitopes, the epitopes are ranked according to their ability to bind to HLA class I/II alleles and the epitopes that are predicted to bind best are selected to be included in the antigenic unit.

Suitable HLA binding algorithms are known in the art.

In yet another embodiment, the antigenic unit comprises multiple T cell epitopes some of which are known to be immunogenic and others that are predicted to be immunogenic. In one embodiment, the T cell epitopes are separated from each other by linkers, e.g. linkers as discussed herein, e.g. as discussed in the “linkers in the antigenic unit” section herein.

Antigenic units comprising T cell epitopes for use in a vector for the prophylactic and therapeutic treatment of betacoronavirus infections and generally applicable methods for selecting T cell epitopes for vectors of the invention used in the prophylactic and therapeutic treatment of infectious diseases are disclosed in detail in WO2021/219897A1, the disclosures of which is incorporated herein by reference.

Antigenic Units of First Polypeptides Comprising One or More Full-Length Infectious Antigens or Parts Thereof or One or More B Cell Epitopes from One or More Pathogens

In another aspect of the invention, a subject, e.g. a human individual, is a healthy individual and the vector of the invention is used prophylactically, e.g. to prevent a disease. Typically, the vector will be used to induce immunity in individuals where it is desired to raise neutralizing antibodies against a pathogen in a prophylactic setting, e.g. to prevent an infection.

In one embodiment, the vector of the invention encodes a first polypeptide that comprises an antigenic unit comprising at least one infectious antigen which is a full-length protein of a pathogen or a part of such a protein. As such, in one embodiment, the at least one infectious antigen is a full-length surface protein or a part thereof, e.g. a full-length viral surface protein or bacterial surface protein or a full-length surface protein of any other pathogen.

In other embodiments, the infectious antigen is a full-length bacterial protein which is secreted by the bacterium, e.g. secreted into the cytoplasm of infected subjects.

In other embodiments, the antigenic unit comprises more than one infectious antigen or parts of more than one infectious antigen, e.g. multiple full-length infectious antigens.

In yet another embodiment, the antigenic unit comprises one or more antigens derived from multiple pathogens or parts of such antigens, e.g. multiple full-lengths infectious antigens from multiple pathogens. In one embodiment, the multiple pathogens are multiple different pathogens.

In one embodiment such a protein of a pathogen is selected from a betacoronavirus protein, e.g. selected from the group consisting of envelope protein, spike protein, membrane protein and, if the betacoronvirus is an Embecovirus, spike-like protein hemagglutinin esterase.

In other embodiments, the antigenic unit comprises one part of one infectious antigen. The RBD domain of the spike protein of SARS-CoV-2 or the head or stem domain of hemagglutinin of the influenza virus are examples of parts of an infectious antigen.

In other embodiments, the antigenic unit comprises several parts of one infectious antigen. In other embodiments, the antigenic unit comprises one part of several infectious antigens, e.g. one part of infectious antigen 1 and one part of infectious antigen 2 and 1 part of infectious antigen 3. In other embodiments, the antigenic unit comprises several parts of several infectious antigens, e.g. 2 parts of infectious antigen 1 and 3 parts of infectious antigen 2. The infectious antigens 1, 2 and 3 may be derived from one pathogen or from multiple, different pathogens

If more than one infectious antigen is comprised in the antigenic unit, or more than 1 part of one or more infectious antigens, the antigens or parts thereof may be separated by linkers, e.g. by linkers as discussed herein, e.g. as discussed in the “linkers in the antigenic unit” section herein.

The one or more infectious antigens or parts thereof comprise conformational B cell epitopes, but may also comprise linear B cell epitopes and/or T cell epitopes. In contrary to the T cell epitopes discussed in the previous section herein, these T cell epitopes are not isolated, but are presented to the immune system in their natural environment, i.e. flanked by the amino acid residues which are present in the antigen.

Thus, in one embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit and an antigenic unit, wherein the antigenic unit comprises one or more full-length infectious antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

In another embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit and an antigenic unit, wherein the antigenic unit comprises one or more full-length antigens derived from one or more pathogens, or parts of such full-length antigens; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the antigenic unit comprises at least a B cell epitope derived from a pathogen, e.g. from a full-length protein of a pathogen, such as a full-length surface protein, e.g. comprised in any of the aforementioned proteins and preferably comprises several B cell epitopes derived from a pathogen, e.g. comprised in a full-length protein of a pathogen, such as a full-length surface protein, e.g. comprised in any of the aforementioned proteins. The at least one B cell epitope may be a linear or a conformational B cell epitope.

In yet another embodiment, the invention provides a vector comprising:
(a) a first nucleic acid sequence encoding a first polypeptide, wherein the first polypeptide comprises a targeting unit that targets antigen-presenting cells, a multimerization unit, such as a dimerization unit and an antigenic unit comprises at least one B cell epitope derived from one or more pathogens; and
(b) one or more further nucleic acid sequences encoding one or more immunostimulatory compounds,
wherein the vector allows for the co-expression of the first polypeptide and the one or more immunostimulatory compounds as separate molecules.

Once administered, the first polypeptide encoded by the first nucleic acid comprised in the vectors of the invention as described above, i.e. comprising an antigenic unit, wherein the antigenic unit comprises one or more infectious full-length antigens or parts of such antigens, elicits a B cell response and T cell response and can be used prophylactic or therapeutic.

Such antigens may be selected for inclusion into the antigenic unit according to their predicted therapeutic efficacy, see WO2021/219897A1, the disclosures of which is incorporated herein by reference.

Antigenic Units of First Polypeptides Comprising B Cell Epitopes and T Cell Epitopes from One or More Pathogens

In one embodiment, the first polypeptide encoded by the first nucleic acid comprised in the vectors of the invention will, once administered to a subject, elicit a T cell response and a B cell response. In a pandemic or an epidemic situation, it is not time efficient to first diagnose an individual to determine if he or she needs primarily a B or T cell response, neither whether prophylactic or therapeutic treatment is the highest medical need. Less so, as the determination of whether or not an individual is infected can be difficult due to lack of (sufficient) applicable tests. Thus, being able to protect and cure at the same time is important. By combining both full-length infectious antigens or parts of infectious antigens or several B cell epitopes present in an infectious antigen and T cell epitopes, such as conserved T cell epitopes, both a strong humoral and cellular response is elicited once the vector is administered. The response can be more humoral or more cellular, depending on the selected targeting unit.

Thus, one aspect of the invention relates to a vector comprising:
(a) a first nucleic acid sequence encoding a first polypeptide, wherein the first polypeptide comprises a targeting unit, multimerization unit, such as a dimerization unit, and an antigenic unit, wherein the antigenic unit comprises (i) one or more full-length infectious antigens or parts of such antigens and (ii) at least one T cell epitope from one or more infectious antigens; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the invention relates to a vector comprising:
(a) a first nucleic acid sequence encoding a first polypeptide, wherein the first polypeptide comprises a targeting unit, a multimerization unit, such as a dimerization unit, and an antigenic unit, wherein the antigenic unit comprises (i) one or more full-length antigens or parts of such antigens and (ii) at least one T cell epitope, wherein the one or more antigens and the at least one T cell epitope are derived from one or more pathogens; and
(b) one or more further nucleic acid sequences encoding one or more immunostimulatory compounds,
wherein the vector allows for the co-expression of the first polypeptide and the one or more immunostimulatory compounds as separate molecules.

Such a combination of T cell epitopes and infectious antigens or parts thereof may be selected for inclusion into the antigenic unit according to the T cell epitopes' predicted immunogenicity or by selecting T cell epitopes known in the art, see WO2021/219897A1, the disclosures of which is incorporated herein by reference.

In one embodiment, the invention relates to a vector comprising:
(a) a first nucleic acid sequence encoding a first polypeptide, wherein the first polypeptide comprises a targeting unit, a multimerization unit, such as a dimerization unit, and an antigenic unit, wherein the antigenic unit comprises (i) one or more B cell epitopes from one or more infectious antigens and (ii) at least one T cell epitope from one or more infectious antigens; and
(b) one or more further nucleic acid sequences encoding one or more immunostimulatory compounds,
wherein the vector allows for the co-expression of the first polypeptide and the one or more immunostimulatory compounds as separate molecules.

In yet another embodiment, the invention relates to a vector comprising:
(a) a first nucleic acid sequence encoding a first polypeptide, wherein the first polypeptide comprises a targeting unit, a multimerization unit, such as a dimerization unit and an antigenic unit, wherein the antigenic unit comprises (i) one or more B cell epitopes and (ii) at least one T cell epitope, wherein the one or more B cell epitopes and the at least one T cell epitope are derived from one or more pathogens; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the full-lengths infectious antigens/parts thereof and the at least one T cell epitope are arranged in the antigenic unit as follows: the at least one T cell epitope is arranged in a subunit which is connected to the multimerization unit by a first linker, such as a unit linker. If multiple T cell epitopes are present in the subunit, the T cell epitopes are preferably separated by subunit linkers. Further, the subunit is separated from the one or more full-length infectious antigens or parts thereof by a second linker. Thus, the subunit with the T cell epitope(s) is closest to the multimerization unit, while the infectious antigen(s) or parts thereof constitute the terminal end of the polypeptide.

Thus, in one embodiment, the invention relates to a vector comprising:
(a) a first nucleic acid sequence encoding a first polypeptide, wherein the first polypeptide comprises a targeting unit, a multimerization unit, such as a dimerization unit, and an antigenic unit, wherein the antigenic unit comprises (i) one or more full-length infectious antigens or parts of such antigens and (ii) one or more T cell epitopes, wherein the one or more antigens and the one or more T cell epitopes are derived from a pathogen; and
(b) one or more further nucleic acid sequences encoding one or more immunostimulatory compounds,
wherein the vector allows for the co-expression of the first polypeptide and the one or more immunostimulatory compounds as separate molecules; and
wherein the antigenic unit comprises a subunit comprising the T cell epitopes which are separated from each other by subunit linkers, if more than one T cell epitope is comprised in the subunit; and
wherein the subunit is connected to the multimerization unit by a first linker, such as a unit linker and separated from the one or more full-length infectious antigens or parts of such antigens by a second linker.

The subunit linkers, first linker/unit linker and second linker may be linkers as discussed herein, e.g. as discussed in the “linkers in the antigenic unit” and “unit linker” sections herein.

### Further Embodiments of the Antigenic Unit

The following applies to the antigenic unit in the first polypeptide encoded by the first nucleic acid comprised in the vectors of the invention in general.

The term antigen is used in this section of the application for a neoantigen, a neoepitope, a patient-present shared cancer antigen, a part of a patient-present shared cancer antigen, such as a patient-present shared cancer epitope, a shared cancer antigen, a part of a shared cancer antigen, such as a shared cancer epitope, an infectious antigen or a part thereof or a T cell epitope of an infectious antigen.

In one embodiment, the antigenic unit comprises only one copy of each antigen. In another embodiment, the antigenic unit comprises multiple copies of one or more antigens.

In one embodiment, the antigenic unit comprises only one copy of each antigen, so that when e.g. 10 different antigens are comprised in the antigenic unit, a vector comprising said antigenic unit may elicit an immune response against all 10 different antigens and thus attack the cancer efficiently.

In another embodiment, if e.g. only a few neoepitopes could be identified in a specific patient that are predicted to be sufficiently immunogenic/predicted to bind to the patient's HLA alleles, then the antigenic unit may comprise at least two copies of a particular antigen, e.g. particular neoepitope, in order to strengthen the immune response to the antigen. If in such patient one or more patient-present shared cancer antigens are identified in addition to such few neoepitopes, it is however preferred to then include such one or more patient-present shared cancer antigens or parts thereof into the antigenic unit rather than including multiple copies of the same neoepitope.

The length of the antigenic unit is determined by the length of the antigen(s) comprised therein as well as their number.

In one embodiment, the antigenic unit comprises up to 3500 amino acids, such as from 60 to 3500 amino acids, e.g. from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

In order to enhance the immune response, particularly for a first polypeptide comprising neoantigens, the antigens may be arranged in the antigenic subunit as described in the following paragraphs.

The antigenic unit can be described as a polypeptide having an N-terminal start and a C-terminal end. The antigenic unit is connected to the multimerization unit, such as dimerization unit, e.g. via a linker, preferably via a unit linker. The antigenic unit is either located at the COOH-terminal end or the NH2-terminal end of the first polypeptide. It is preferred that the antigenic unit is in the COOH-terminal end of the first polypeptide.

In one embodiment, the antigens, preferably epitopes, are arranged in the order of more antigenic to less antigenic in the direction from the N-terminal start of the antigenic unit to the C-terminal end of the antigenic unit. Alternatively, particularly if the hydrophilicity/hydrophobicity varies greatly among the antigens, it is preferred that the most hydrophobic antigens is/are substantially positioned in the middle of the antigenic unit and the most hydrophilic antigens is/are positioned at the N-terminal start and/or the C-terminal end of the antigenic unit.

Since a true positioning in the middle of the antigenic unit is only possible if the antigenic unit comprises an odd number of antigens, the term “substantially” in this context refers to antigenic units comprising an even number of antigens, wherein the most hydrophobic antigens are positioned as closed to the middle as possible.

By way of example, an antigenic unit comprises 5 antigenic subunits, each comprising a different epitope, e.g. a different neoepitope, which are arranged as follows: 1-2-3*-4-5; with 1, 2, 3*,4 and 5 each being a different neoepitope and - being a subunit linker and * indicating the most hydrophobic neoepitope, which is positioned in the middle of the antigenic unit.

In another example, an antigenic unit comprises 6 antigenic subunits, each comprising a different epitope, e.g. a different neoepitope, which are arranged as follows: 1-2-3*-4-5-6 or, alternatively, as follows: 1-2-4-3*-5-6; with 1, 2, 3*, 4, 5 and 6 each being a different neoepitope and - being a subunit linker and * indicating the most hydrophobic neoepitope, which is positioned substantially in the middle of the antigenic unit.

Alternatively, the antigenic subunits may be arranged such that they alternate between a hydrophilic and a hydrophobic antigen.

Optionally, GC rich sequences encoding antigens (e.g. GC rich sequences encoding neoepitopes or epitopes) are arranged in such a way, that GC clusters are avoided. In one embodiment, GC rich sequences encoding for antigens are arranged such that there is at least one non-GC rich sequence between them.

In one embodiment, the antigenic unit comprises one or more linkers. In another embodiment, the antigenic unit comprises multiple antigens, e.g. multiple epitopes, e.g. neoepitopes, wherein the antigens are separated by linkers. In yet another embodiment, the antigenic unit comprises multiple antigens wherein each antigen is separated from other antigens by linkers. An alternative way to describe the separation of each antigen from other antigens by linkers is that all but the terminal antigen, i.e. the antigen at the N-terminal start of the polypeptide or the C-terminal end of the polypeptide (i.e. located at the end of the antigenic unit that is not connected to the multimerization unit), are arranged in antigenic subunits, wherein each subunit comprises or consists of an antigen e.g. a neoepitope, and a subunit linker.

Hence, an antigenic unit comprising n antigens comprises n−1 antigenic subunits, wherein each subunit comprises an antigen and a subunit linker, and further comprises a terminal antigen. In one embodiment, wherein n is an integer of from 1 to 50, e.g. 3 to 50 or 15 to 40 or 10 to 30 or 10 to 25 or 10 to 20 or 15 to 30 or 15 to 25 or 15 to 20.

Due to the separation of the antigens by the linkers, each antigen is presented in an optimal way to the immune system.

In one embodiment, the antigenic unit comprises B cell epitopes and T cell epitopes, e.g. a full-length infectious antigen or part thereof and one or more T cell epitopes comprised in a protein of a pathogen and the antigenic unit is designed such that the T cell epitopes are arranged closest to the multimerization unit and the infectious antigen is at the terminal end of the antigenic unit. The T cell epitopes are preferably separated by linkers and the infectious antigen is preferably separated from the “subunit” comprising the T cell epitopes by a linker. Such afore-mentioned antigenic unit designs are disclosed in PCT/EP2022/061819, the disclosures of which is incorporated herein by reference

### Linkers Comprised in the Antigenic Unit

The antigenic unit may comprise linkers, e.g. linkers that separate the antigens comprised therein, e.g. neoantigens, neoepitopes, patient-present shared cancer antigens or parts thereof, such as patient-present shared cancer epitopes, shared cancer antigen or parts thereof, such as shared cancer epitopes, infectious antigens or parts thereof or T cell epitopes of an infectious antigen. As described above, all antigens, such as neoepitopes, may be separated from each other by linkers and arranged in subunits. In the following, the term subunit linker and linker are used interchangeably, and both denote a linker in the antigenic unit.

In one embodiment, the linkers are designed to be non-immunogenic. A linker may be a rigid linker, meaning that that it does not allow the two amino acid sequences that it connects to substantially move freely relative to each other. Alternatively, it may be a flexible linker, i.e. a linker that allows the two amino acid sequences that it connects to substantially move freely relative to each other. Both types of linkers are useful. In one embodiment, the linker is a flexible linker, which allows for presenting the antigen in an optimal manner to the T cells, even if the antigenic unit comprises a large number of antigens.

In one embodiment, the subunit linker is a peptide consisting of from 4 to 40 amino acids, e.g. 35, 30, 25 or 20 amino acids, e.g. from 5 to 20 amino acids or 5 to 15 amino acids or 8 to 20 amino acids or 8 to 15 amino acids 10 to 15 amino acids or 8 to 12 amino acids. In another embodiment, the subunit linker consists of 10 amino acids.

In one embodiment, e.g. in an antigenic unit comprising neoepitopes, the subunit linker is identical in all antigenic subunits. If, however, one or more of the antigens comprise a sequence similar to that of the linker, it may be an advantage to substitute the neighboring subunit linkers with a linker of a different sequence. Also, if an antigen-subunit linker junction is predicted to constitute an immunogenic epitope in itself, then a linker of a different sequence may be used.

In one embodiment, the subunit linker is a flexible linker, preferably a flexible linker which comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In one embodiment, the flexible linker is a serine(S) and/or glycine (G) rich linker, i.e. a linker comprising several serine and/or several glycine residues. Preferred examples are GGGGS (SEQ ID NO: 58), GGGSS (SEQ ID NO: 59), GGGSG (SEQ ID NO: 60), GGSGG (SEQ ID NO: 61), SGSSGS (SEQ ID NO: 62) or multiple variants thereof such as GGGGSGGGGS (SEQ ID NO: 17), (GGGGS)m (SEQ ID NO: 64), (GGGSS)m (SEQ ID NO: 65), (GGSGG)m (SEQ ID NO: 66), (GGGSG)m (SEQ ID NO: 67) or (SGSSGS)m (SEQ ID NO: 68), where m is an integer from 1 to 5, e.g., 1, 2, 3, 4, or 5. In a preferred embodiment, m is 2. In another preferred embodiment, the serine and/or glycine rich linker further comprises at least one leucine (L) residue, such as at least 1 or at least 2 or at least 3 leucine residues, e.g. 1, 2, 3 or 4 leucine residues.

In one embodiment, the subunit linker comprises or consists of LGGGS (SEQ ID NO: 69), GLGGS (SEQ ID NO: 70), GGLGS (SEQ ID NO: 71), GGGLS (SEQ ID NO: 72) or GGGGL (SEQ ID NO: 73). In another embodiment, the subunit linker comprises or consists of LGGSG (SEQ ID NO: 74), GLGSG (SEQ ID NO: 75), GGLSG (SEQ ID NO: 76), GGGLG (SEQ ID NO: 77) or GGGSL (SEQ ID NO: 78). In yet another embodiment, the subunit linker comprises or consists of LGGSS (SEQ ID NO: 79), GLGSS (SEQ ID NO: 80) or GGLSS (SEQ ID NO: 81).

In yet another embodiment, the subunit linker comprises or consists of LGLGS (SEQ ID NO: 85), GLGLS (SEQ ID NO: 86), GLLGS (SEQ ID NO: 87), LGGLS (SEQ ID NO: 88) or GLGGL (SEQ ID NO: 89). In yet another embodiment, the subunit linker comprises or consists of LGLSG (SEQ ID NO: 90), GLLSG (SEQ ID NO: 91), GGLSL (SEQ ID NO: 92), GGLLG (SEQ ID NO: 93) or GLGSL (SEQ ID NO: 94). In yet another embodiment, the subunit linker comprises or consists of LGLSS (SEQ ID NO: 95), or GGLLS (SEQ ID NO: 96).

In another embodiment, the subunit linker is serine-glycine linker that has a length of 10 amino acids and comprises 1 or 2 leucine residues.

In one embodiment, the subunit linker comprises or consists of LGGGSGGGGS (SEQ ID NO: 97), GLGGSGGGGS (SEQ ID NO: 98), GGLGSGGGGS (SEQ ID NO: 99), GGGLSGGGGS (SEQ ID NO: 100) or GGGGLGGGGS (SEQ ID NO: 101). In another embodiment, the subunit linker comprises or consists of LGGSGGGGSG (SEQ ID NO: 102), GLGSGGGGSG (SEQ ID NO: 103), GGLSGGGGSG (SEQ ID NO: 104), GGGLGGGGSG (SEQ ID NO: 105) or GGGSLGGGSG (SEQ ID NO: 106). In yet another embodiment, the subunit linker comprises or consists of LGGSSGGGSS (SEQ ID NO: 107), GLGSSGGGSS (SEQ ID NO: 1008), GGLSSGGGSS (SEQ ID NO: 109), GGGLSGGGSS (SEQ ID NO: 110) or GGGSLGGGSS (SEQ ID NO: 111).

In a further embodiment, the subunit linker comprises or consists of LGGGSLGGGS (SEQ ID NO: 112), GLGGSGLGGS (SEQ ID NO: 113), GGLGSGGLGS (SEQ ID NO: 114), GGGLSGGGLS (SEQ ID NO: 115) or GGGGLGGGGL (SEQ ID NO: 116). In another embodiment, the subunit linker comprises or consists of LGGSGLGGSG (SEQ ID NO: 117), GLGSGGLGSG (SEQ ID NO: 118), GGLSGGGLSG (SEQ ID NO: 119), GGGLGGGGLG (SEQ ID NO: 120) or GGGSLGGGSL (SEQ ID NO: 121). In yet another embodiment, the subunit linker comprises or consists of LGGSSLGGSS (SEQ ID NO: 122), GLGSSGLGSS (SEQ ID NO: 123) or GGLSSGGLSS (SEQ ID NO: 124).

In yet another embodiment, the subunit linker comprises or consists of GSGGGA (SEQ ID NO: 125), GSGGGAGSGGGA (SEQ ID NO: 126), GSGGGAGSGGGAGSGGGA (SEQ ID NO: 127), GSGGGAGSGGGAGSGGGAGSGGGA (SEQ ID NO: 128) or GENLYFQSGG (SEQ ID NO: 129). In yet another embodiment, the subunit linker comprises or consists of SGGGSSGGGS (SEQ ID NO: 130), SSGGGSSGGG (SEQ ID NO: 131), GGSGGGGSGG (SEQ ID NO: 132), GSGSGSGSGS (SEQ ID NO: 133), GGGSSGGGSG (SEQ ID NO: 134) (amino acids 121-130 of SEQ ID NO: 1), GGGSSS (SEQ ID NO: 135), GGGSSGGGSSGGGSS (SEQ ID NO: 136) or GLGGLAAA (SEQ ID NO: 137).

In another embodiment, the subunit linker is a rigid linker. Such rigid linkers may be useful to efficiently separate (larger) antigens and prevent their interferences with each other. In one embodiment, the subunit linker comprises or consist of KPEPKPAPAPKP (SEQ ID NO: 138), AEAAAKEAAAKA (SEQ ID NO: 139), (EAAAK)m (SEQ ID NO: 140), PSRLEEELRRRLTEP (SEQ ID NO: 141) or SACYCELS (SEQ ID NO: 142).

In yet another embodiment, the subunit linker comprises or consists of TQKSLSLSPGKGLGGL (SEQ ID NO: 143). In yet another embodiment, the subunit linker comprises or consists of SLSLSPGKGLGGL (SEQ ID NO: 144).

In yet another embodiment, the subunit linker comprises or consists of GGSAGGSGSGSSGGSSGASGTGTAGGTGSGSGTGSG (SEQ ID NO: 145); or GGSGGGSEGGGSEGGGSEGGGSEGGGSEGGGSGGGS (SEQ ID NO: 146) or ELKTPLGDTTHT (SEQ ID NO: 147) (amino acids 94-105 of SEQ ID NO: 1) or EPKSCDTPPPCPRCP (SEQ ID NO: 148) (amino acids 106-120 of SEQ ID NO: 1).

In yet another embodiment, the subunit linker is a cleavable linker, e.g. a linker which includes one or more recognition sites for endopeptidases, e.g. endopeptidases such as furin, caspases, cathepsins and the like. Cleavable linkers may be introduced to release free functional protein domains (e.g. encoded by larger antigens), which may overcome steric hindrance between such domains or other drawbacks due to interference of such domains, like decreased bioactivity, altered biodistribution.

Examples of suitable linkers are disclosed in paragraphs [0098]-[0099] and in the recited sequences of WO 2020/176797A1, in paragraphs to of US 2019/0022202A1, in WO 2017/118695 A1 and in WO 2021/219897A1, all of which are incorporated herein by reference.

### Unit Linker

The antigenic unit is connected to the multimerization unit, preferably by a unit linker. Thus, in one embodiment, the first nucleic acid sequence comprised in the vectors of the invention encodes a first polypeptide that comprises a unit linker that connects the antigenic unit to the multimerization unit.

The unit linker may comprise a restriction site in order to facilitate the construction of the first nucleic acid sequence. In one embodiment, the unit linker is GLGGL (SEQ ID NO: 89) or GLSGL (SEQ ID NO: 149). In another embodiment, the unit linker comprises or consists of GGGGS (SEQ ID NO: 58), GGGGSGGGGS (SEQ ID NO: 17), (GGGGS)m (SEQ ID NO: 64), EAAAK (SEQ ID NO: 150), (EAAAK)m (SEQ ID NO: 140), (EAAAK) mGS (SEQ ID NO: 151), (EAAK) mGS (SEQ ID NO: 63), GPSRLEEELRRRLTEPG (SEQ ID NO: 152), AAY or HEYGAEALERAG (SEQ ID NO: 153).

### Signal Peptide

In one embodiment of the present disclosure, at least one of the first nucleic acid sequence or the one or more further nucleic acid sequences encoding one or more immunostimulatory compounds also encodes a signal peptide. The signal peptide is either located at the N-terminal end of the targeting unit or the C-terminal end of the targeting unit, depending on the orientation of the targeting unit in the first polypeptide. Further, the signal peptide is located at the N-terminal end of the immunostimulatory compound. The signal peptide is designed to allow secretion of the first polypeptide/immunostimulatory compound(s) from cells comprising a vector of the invention. Preferably, the first nucleic acid sequence and each of the further nucleic acid sequences encoding one or more immunostimulatory compounds also encode a signal peptide. Preferably, the signal peptide is that which is naturally present at the N-terminus of any of the targeting units or immunostimulatory compounds described herein.

Any suitable signal peptide may be used. Examples of suitable peptides are an Ig VH signal peptide, preferably a human Ig VH signal peptide, such as SEQ ID NO: 2, preferably if the targeting unit is an antibody or part thereof, such as a scFv. In one embodiment, the signal peptide is the natural leader sequence of the protein which is the targeting unit, i.e. the signal peptide which is naturally present at the N-terminus of any of the protein which is encoded in the vector of the invention as the targeting unit. In another embodiment, the signal peptide is the natural leader sequence of the immunostimulatory compound, i.e. the signal peptide which is naturally present at the N-terminus of the protein which is the immunostimulatory compound.

Examples of signal peptides are a human TPA signal peptide, such as SEQ ID NO: 3, a human MIP1-α signal peptide, such as the amino acid sequence 1-23 of SEQ ID NO: 1, a human GM-CSF signal peptide, such as the amino acid sequence of SEQ ID NO: 40, a human CCL5 signal peptide, such as the amino acid sequence of SEQ ID NO: 42, a human IL-12A signal peptide, such as the amino acid sequence of SEQ ID NO: 44, a human IL-12B signal peptide, such as the amino acid sequence of SEQ ID NO: 46 or a human IL-21 signal peptide, such as the amino acid sequence of SEQ ID NO: 48.

In a preferred embodiment, the vectors of the invention comprise a first nucleotide sequence encoding a first polypeptide and further encoding a signal peptide that comprises an amino acid sequence having at least 85% sequence identity to the amino acid sequence 1-23 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In another preferred embodiment, the vectors of the invention comprise a first nucleotide sequence a first polypeptide and further encoding a signal peptide that comprises the amino acid sequence 1-23 of SEQ ID NO: 1, except that at the most three amino acids have been substituted, deleted or inserted, such as at the most two amino acids or such as at the most one amino acid.

In another preferred embodiment, the vectors of the invention comprise a first nucleotide sequence encoding a first polypeptide and further encoding a signal peptide that comprises the amino acid sequence 1-23 of SEQ ID NO: 1.

In a more preferred embodiment, the vectors of the invention comprise a first nucleotide sequence encoding a first polypeptide and further encoding a signal peptide that consists of an amino acid sequence having at least 85% sequence identity to the amino acid sequence 1-23 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In another preferred embodiment, the vectors of the invention comprise a first nucleotide sequence encoding a first polypeptide and further encoding a signal peptide that consists of the amino acid sequence 1-23 of SEQ ID NO: 1, except that at the most three amino acids have been substituted, deleted or inserted, such as at the most two amino acids or such as at the most one amino acid.

In another preferred embodiment, the vectors of the invention comprise a first nucleotide sequence encoding a first polypeptide and further encoding a signal peptide with the amino acid sequence 1-23 of SEQ ID NO: 1.

In one preferred embodiment, the vectors of the invention comprise a first nucleotide sequence encoding a first polypeptide and further encoding a signal peptide, wherein said nucleotide sequence of said signal peptide has at least 80% sequence identity to the nucleic acid sequence with SEQ ID NO: 29.

In a further preferred embodiment, the vectors of the invention comprise a first nucleotide sequence encoding a first polypeptide and further encoding a signal peptide, wherein said nucleotide sequence of said signal peptide has at least 85% sequence identity to the nucleic acid sequence with SEQ ID NO: 29, such as at least 86% or at least 87%, 88%, 89%, 90%, 91%, 92%, 93%, 94%, 95%, 96%, 97%, 98% or at least 99% sequence identity.

In yet a further preferred embodiment, the vectors of the invention comprise a first nucleotide sequence encoding a first polypeptide and further encoding a signal peptide, wherein said nucleotide sequence of said signal peptide is SEQ ID NO: 29.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

Amino acid sequence variants may be prepared by introducing appropriate changes into the nucleotide sequence encoding the first polypeptide and/or one or more immunostimulatory compounds, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the final first polypeptide and/or one or more immunostimulatory compounds, provided that the final proteins have the desired characteristics. For example, deletions, insertions or substitutions of amino acid residues may produce a silent change and result in a functionally equivalent polypeptide/immunostimulatory compound.

Deliberate amino acid substitutions may be made on the basis of similarity in polarity, charge, solubility, hydrophobicity, hydrophilicity, and/or the amphipathic nature of the residues as long as the desired properties of the protein in question are retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitution such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, valine, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above, * indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or B-alanine residues. A further form of variation involves the presence of one or more amino acid residues in peptoid form.

### Polypeptides and Multimeric/Dimeric Proteins

The vectors of the invention encode a first polypeptide as described above. The polypeptide (and the one or more immunostimulatory compounds) are expressed in vivo as a result of the administration of the vector to a subject.

Due to the presence of the multimerization unit, such as dimerization unit, multimeric proteins are formed when the polypeptide is expressed.

The multimeric proteins may be homomultimers or hetereomultimers, e.g. if the protein is a dimeric protein, the dimeric protein may be a homodimer, i.e. a dimeric protein wherein the two polypeptide chains are identical and consequently comprise identical units and thus antigen sequences, or the dimeric protein may be a heterodimer comprising two polypeptide chains, wherein polypeptide chain 1 comprises different antigen sequences in its antigenic unit than polypeptide 2. The latter may be relevant if the number of antigens for inclusion into the antigenic unit would exceed an upper size limit for the antigenic unit. It is preferred that the multimeric protein is a homomultimeric protein.

### Production of the Vector and Host Cells

The vectors of the invention are generally vectors suitable for transfecting a host cell and a) expression of the first polypeptide and formation of a multimeric protein comprised of multiple of such first polypeptides encoded by the first nucleic acid sequence and b) expression of the one or more immunostimulatory compounds encoded by the further nucleic acid sequences, respectively.

In one embodiment, the host cell comprising the vector of the invention is a cell of a cell culture, e.g. a bacteria cell, and the proteins encoded by the vector are expressed in vitro. In another embodiment, the host cell comprising the vector of the invention is a cell of a subject and the proteins encoded by the vector are expressed in said subject, i.e. in vivo, as a result of the administration of the vector to a subject.

Suitable host cells for in vitro transfection include prokaryote cells, yeast cells, insect cells or higher eukaryotic cells. Suitable host cells for in vivo transfection are e.g. muscle cells.

In one embodiment, the vectors allows for easy exchange of the various units described above, particularly the antigenic unit in case of individualized antigenic units. In one embodiment, the vector is a pUMVC4a vector or a vector comprising NTC9385R vector backbones. The antigenic unit may be exchanged with an antigenic unit cassette restricted by the Sfil restriction enzyme cassette where the 5′ site is incorporated in the nucleotide sequence encoding the GLGGL (SEQ ID NO: 89)/GLSGL (SEQ ID NO: 149) unit linker and the 3′ site is included after the stop codon in the vector.

Engineering and production methods of the vectors of the invention, e.g. expression vectors such as DNA and RNA plasmids or viral vectors are well known and the skilled person will be able to engineer/produce the vectors of the invention using such known methods. Moreover, various commercial manufacturers offer services for vector design and production.

In one aspect, the disclosure relates to a method of producing a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof; and
(b) one or more further nucleic acid sequences encoding one or more immunostimulatory compounds,
wherein the vector allows for the co-expression of the first polypeptide and the one or more immunostimulatory compounds as separate molecules, the method comprising:
   a) transfecting cells in vitro with the vector;
   b) culturing said cells;
   c) optionally, lysing the cells to release the vector from the cells; and
   d) collecting and optionally purifying the vector.

In one embodiment, the one or more antigens or parts thereof are disease-relevant antigens or parts thereof.

### Pharmaceutical Compositions

In one embodiment of the present disclosure, the vector, e.g. DNA plasmid is for use as a medicament.

Thus, in one embodiment of the present disclosure, the vector is provided in a pharmaceutical composition comprising the vector and a pharmaceutically acceptable carrier or diluent.

Thus, in one aspect, the disclosure relates to a pharmaceutical composition comprising (i) a pharmaceutically acceptable carrier or diluent and (ii) a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

In one embodiment, the one or more antigens or parts thereof are disease-relevant antigens or parts thereof.

Suitable pharmaceutically acceptable carriers or diluents include, but are not limited to, saline, buffered saline, such as PBS, dextrose, water, glycerol, ethanol, isotonic aqueous buffers, and combinations thereof.

In one embodiment, the pharmaceutically acceptable carrier or diluent is an aqueous buffer. In another embodiment, the aqueous buffer is Tyrode's buffer, e.g. Tyrode's buffer comprising 140 mM NaCl, 6 mM KCl, 3 mM CaCl2, 2 mM MgCl2, 10 mM 4-(2-hydroxyethyl)-1-piperazineethanesulfonic acid (Hepes) pH 7.4, and 10 mM glucose.

The pharmaceutical composition may comprise molecules that ease the transfection of host cells, i.e. a transfection agent.

In some specific embodiments pharmaceutical composition comprises a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO) 4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In one embodiment, the pharmaceutical composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In another embodiment, the pharmaceutical composition comprises the amphiphilic block co-polymer in an amount of from 2% w/v to 5% w/v, such as about 3% w/v.

The pharmaceutical composition may be formulated in any way suitable for administration to a subject, e.g. such as a liquid formulation for injection, e.g. for intradermal or intramuscular injection.

The pharmaceutical composition may be administered in any way suitable for administration to a subject, such as administered by intradermal, intramuscular, or subcutaneous injection, or by mucosal or epithelial application, such as intranasal or oral.

In a preferred embodiment, the pharmaceutical composition is administered by intramuscular or intradermal injection.

The amount of vector, e.g. DNA plasmid, in the pharmaceutical composition may vary depending on whether the pharmaceutical composition is administered for prophylactic or therapeutic treatment.

The pharmaceutical composition of the invention typically comprises the vector, e.g. DNA plasmid, in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg.

In a preferred embodiment, the pharmaceutical composition is a sterile pharmaceutical composition.

### Treatment

In some aspects of the present disclosure, the vector, e.g. DNA plasmid, is for use in the therapeutic or prophylactic treatment of a disorder, such as a disorder in humans.

Thus, in one aspect, the disclosure relates to a method of treating a subject having a disease or being in need of prevention of said disease, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, which are relevant for said disease; and
(b) one or more further nucleic acid sequences encoding one or more immunostimulatory compounds,
wherein the vector allows for the co-expression of the first polypeptide and the one or more immunostimulatory compounds as separate molecules.

In the method of treatment, the vector is preferably administered in a therapeutically effective or prophylactically effective amount. Such amount of vector may be administered in one administration, i.e. one dose, or in several administrations, i.e. repetitive doses, i.e. in a series of doses, e.g. over the course of several days, weeks or months.

The actual dose to be administered may vary and depend on whether the treatment is a prophylactic or therapeutic treatment, the age, weight, gender, medical history, pre-existing conditions and general condition of the subject, the severity of the disease being treated and the judgment of the health care professionals.

In the method of treatment, the vector may be administered in the form of the pharmaceutical composition and in the mode of administration as described herein.

The method of treating according to the invention can continue for as long as the clinician overseeing the patient's care deems the method to be effective and the treatment to be needed.

In one embodiment of the present disclosure, the vector, e.g. DNA plasmid, is for use in the treatment of a cancer. Such vectors and antigenic units of such vectors, including antigenic units of individualized and non-individualized first polypeptides and various embodiments thereof, have been described in detail herein.

Thus, in one embodiment, the disclosure relates to a method of treating a subject having cancer, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

The cancer may be a solid or a liquid cancer. Examples of solid cancers are cancers forming a solid mass, e.g. a tumor. Examples of liquid cancers are cancers present in body fluid, such as lymphomas or blood cancers.

In one embodiment of the present disclosure, the vector, e.g. DNA plasmid is for use in the treatment of a cancer selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

In another embodiment of the present disclosure, the vector, e.g. DNA plasmid, is for use in the treatment of an infectious disease. Such vectors and antigenic units of such vectors have been described in detail herein.

Thus, in one embodiment, the disclosure relates to a method of treating a subject having an infectious disease or being in need of prevention of an infectious disease, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, which are relevant for said infectious disease; and
(b) one or more further nucleic acid sequences encoding one or more immunostimulatory compounds,
wherein the vector allows for the co-expression of the first polypeptide and the one or more immunostimulatory compounds as separate molecules.

Antigens or parts thereof which are relevant for infectious diseases, e.g. which are derived from pathogens, have been described in detail herein.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, which are relevant for a disease; and
(b) one or more further nucleic acid sequences encoding one or more immunostimulatory compounds,
wherein the vector allows for the co-expression of the first polypeptide and the one or more immunostimulatory compounds as separate molecules for use in the treatment of a subject having said disease or being in need of prevention of said disease, wherein said vector is administered to said subject.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules, for use in the treatment of a subject having cancer, wherein said vector is administered to said subject.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, which are relevant for an infectious disease; and
(b) one or more further nucleic acid sequences encoding one or more immunostimulatory compounds,
wherein the vector allows for the co-expression of the first polypeptide and the one or more immunostimulatory compounds as separate molecules, for use in the treatment of a subject having said infectious disease or being in need of prevention of said infectious disease, wherein said vector is administered to said subject.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, which are relevant for a disease; and
(b) one or more further nucleic acid sequences encoding one or more immunostimulatory compounds,
wherein the vector allows for the co-expression of the first polypeptide and the one or more immunostimulatory compounds as separate molecules, for the manufacture of a medicament for use in the treatment of a subject having said disease or is in need of prevention of said disease wherein said medicament is administered to said subject.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules, for the manufacture of a medicament for use in the treatment of a subject having cancer wherein said medicament is administered to said subject.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for an infectious disease; and
(b) one or more further nucleic acid sequences encoding one or more immunostimulatory compounds,
wherein the vector allows for the co-expression of the first polypeptide and the one or more immunostimulatory compounds as separate molecules, for the manufacture of a medicament for use in the treatment of a subject having said infectious disease or being in need of prevention of said infectious disease, wherein said medicament is administered to said subject.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof relevant for a disease; and
(b) one or more further nucleic acid sequences encoding one or more immunostimulatory compounds,
wherein the vector allows for the co-expression of the first polypeptide and the one or more immunostimulatory compounds as separate molecules, for treating a subject having said disease or being in need of prevention of said disease.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules, for treating a subject having cancer.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for an infectious disease; and
(b) one or more further nucleic acid sequences encoding one or more immunostimulatory compounds,
wherein the vector allows for the co-expression of the first polypeptide and the one or more immunostimulatory compounds as separate molecules, for treating a subject having said infectious disease or being in need of prevention of said infectious disease.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof relevant for a disease; and
(b) one or more further nucleic acid sequences encoding one or more immunostimulatory compounds,
wherein the vector allows for the co-expression of the first polypeptide and the one or more immunostimulatory compounds as separate molecules, when used in the therapeutic or prophylactic treatment of said disease.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules, when used in treatment of cancer.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for an infectious disease; and
(b) one or more further nucleic acid sequences encoding one or more immunostimulatory compounds,
wherein the vector allows for the co-expression of the first polypeptide and the one or more immunostimulatory compounds as separate molecules, when used in the therapeutic or prophylactic treatment of said infectious disease.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for a disease; and
(b) one or more further nucleic acid sequences encoding one or more immunostimulatory compounds,
wherein the vector allows for the co-expression of the first polypeptide and the one or more immunostimulatory compounds as separate molecules, for the therapeutic or prophylactic treatment of said disease.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules, for the treatment of cancer.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for an infectious disease; and
(b) one or more further nucleic acid sequences encoding one or more immunostimulatory compounds,
wherein the vector allows for the co-expression of the first polypeptide and the one or more immunostimulatory compounds as separate molecules, for the therapeutic or prophylactic treatment of said infectious disease.

Also disclosed herein is a medicament for the treatment or prevention of a disease in a subject having said disease or being in need of prevention of said disease by administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for said disease; and
(b) one or more further nucleic acid sequences encoding one or more immunostimulatory compounds,
wherein the vector allows for the co-expression of the first polypeptide and the one or more immunostimulatory compounds as separate molecules.

Also disclosed herein is a medicament for the treatment of cancer in a subject having cancer by administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more cancer antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

Also disclosed herein is a medicament for the treatment or prevention of an infectious disease in a subject having said disease or being in need of prevention of said disease by administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof which are relevant for said disease; and
(b) one or more further nucleic acid sequences encoding one or more immunostimulatory compounds,
wherein the vector allows for the co-expression of the first polypeptide and the one or more immunostimulatory compounds as separate molecules.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1

Various DNA plasmids were designed which allow the co-expression of a first polypeptide as described herein, and one or more immunostimulatory compounds, as separate molecules.

All DNA plasmids, VB4194, VB4168, VB4169 and VB4170, comprise nucleic acid sequences encoding the elements/units listed in Table 4 below:

TABLE 4

| Elements/Units | Description and sequence |
|---|---|
| Signal peptide | Natural leader sequence for human MIP-1α |
| | Amino acids 1-23 of SEQ ID NO: 1 |
| Targeting unit | Full-length human MIP-1 sequence |
| | Amino acids 24-93 of SEQ ID NO: 1 |
| Dimerization unit | Human hinge-region 1 from IgG |
| | Amino acids 94-105 of SEQ ID NO: 1 |
| | Human hinge-region 4 from IgG3 |
| | Amino acids 106-120 of SEQ ID NO: 1 |
| | Glycine-serine-linker |
| | Amino acids 121-130 of SEQ ID NO: 1 |
| | Human CH3 domain from IgG3 |
| | Amino acids 131-237 of SEQ ID NO: 1 |
| Unit linker | Glycine-leucine linker |
| | SEQ ID NO: 89 |

The DNA plasmids further comprise nucleic acid sequences encoding the elements/units listed in Table 5 below:

TABLE 5

| Elements/ | VB4194 | VB4168 | VB4169 | VB4170 |
|---|---|---|---|---|
| Units | SEQ ID NO: 5 | SEQ ID NO: 6 | SEQ ID NO: 7 | SEQ ID NO: 8 |
| Antigenic unit | See Table 6 below | | | |
| Co-expression | NA | T2A | | |
| element | | SEQ ID NO: 9 | | |
| Signal peptide | NA | Natural leader sequence for human FLT3L | | |
| | | SEQ ID NO: 4 | | |
| Immunostimulatory | NA | Human FLT3L | | |
| compound 1 | | SEQ ID NO: 10 | | |
| Co-expression | NA | NA | P2A | |
| element | | | SEQ ID NO: 11 | |
| Signal peptide | NA | NA | Natural leader sequence for | |
| | | | mouse GM-CSF | |
| | | | SEQ ID NO: 12 | |
| Immunostimulatory | NA | NA | Mouse GM-CSF | |
| compound 2 | | | SEQ ID NO: 13 | |
| Co-expression | NA | NA | NA | E2A |
| element | | | | SEQ ID NO: 14 |
| Signal peptide | NA | NA | NA | Natural leader |
| | | | | sequence for |
| | | | | mouse CCL5 |
| | | | | SEQ ID NO: 15 |
| Immunostimulatory | NA | NA | NA | Mouse CCL5 |
| compound 3 | | | | SEQ ID NO: 16 |

In the following, “m”, “murine” and “mouse” are used interchangeably, and “h” and human are used interchangeably.

DNA Plasmids VB4194, VB4168, VB4169 and VB4170 Comprising 8 Epitopes with Mutations:

Previously described exome sequencing and RNA sequencing of the mouse colon cancer cell line CT26 revealed hundreds to thousands of tumor-specific non-synonymous mutations. In silico methods were used to identify potential immunogenic sequences, i.e. epitopes comprising a mutation, and 8 of them (Table 6) were chosen for inclusion into the antigenic unit of the first polypeptide encoded by the above-mentioned DNA plasmids. The epitopes in said antigenic unit are separated by glycine-serine linkers (GGGGSGGGGS, SEQ ID NO: 17), i.e. all epitopes but the terminal epitope are arranged in subunits, each subunit consisting of one epitope and one GGGGGGGGS (SEQ ID NO: 17) linker.

Each of these DNA plasmids is a model of a DNA plasmid according to the invention encoding for an individualized first polypeptide, i.e. one that comprises an antigenic unit comprising several patient-specific epitopes, e.g. several neoepitopes and/or several patient-present shared cancer epitopes, with the patient-present shared cancer antigens being mutated patient-present shared cancer antigens, or a model of a DNA plasmid according to the invention encoding for a non-individualized first polypeptide, i.e. one that comprises an antigenic unit comprising several shared cancer epitopes, with the shared cancer antigens being mutated shared cancer antigens.

The DNA plasmids VB4168, VB4169 and VB4170 allow the co-expression of a first polypeptide as described above and in Tables 4 and 5 and the following immunostimulatory compounds, as separate molecules (h=human; m=mouse):
VB4194: encodes only a first polypeptide (the same first polypeptide as VB4168, VB4169 and VB4170), no immunostimulatory compound and serves as a comparison.
VB4168: hFLT3L.
VB4169: hFLTL3 and mGM-CSF.
VB4170: hFLTL3, mGM-CSF and mCCL5

TABLE 6

| | | | SEQ |
|---|---|---|---|
| Epitope | Gene | Sequence | ID NO: |
| C-pepM1 | E2f8 | VILPQAPSGPSYAT | 154 |
| | | YLQPAQAQMLTPP | |
| C-pepM8 | Dhx35 | EVIQTSKYYMRDVI | 155 |
| | | AIESAWLLELAPH | |
| C-pepM43 | Mtch1 | KSWIHCWKYLSVQS | 156 |
| | | QLFRGSSLLFRRV | |
| C-pepM69 | Klhl28 | GDVKIHAHKVVLAN | 157 |
| | | ISPYFKAMFTGNL | |
| C-pepM149 | 3110057O12Rik | FVSPMAHYVPGIMA | 158 |
| | | IESVVARFQFIVP | |
| C-pepM173 | Top3a | KIYEFDYHLYGQNI | 159 |
| | | TMIMTSVSGHLLA | |
| C-pepM174 | Cltc | NNLQKYIEIYVQKI | 160 |
| | | NPSRLPVVIGGLL | |
| C-pepM175 | Mms22I | TPLRKHTVHAIRKF | 161 |
| | | YLEFKGSSPPPRL | |

### Production of DNA Plasmids

The sequences of the antigenic units, co-expression elements and immunostimulatory compounds of all DNA plasmids disclosed in the Examples in the were ordered from Genscript (Genscript Biotech B.V., Netherlands) and cloned into the expression vector pUMVC4a; a master plasmid comprising a nucleotide sequence encoding the signal peptide, targeting unit, dimerization unit and unit linker described in Table 4 above.

### Assessment of Expression and Secretion of Proteins Encoded by DNA Plasmids

HEK293 cells (ATCC) were transiently transfected with the above-mentioned DNA plasmids. Briefly, 2×10⁵ cells/well were plated in 24-well tissue culture plates with 10% FBS growth medium and transfected with 1 μg of respective DNA plasmid using Lipofectamine® 2000 reagent under the conditions suggested by the manufacturer

(Invitrogen, Thermo Fischer Scientific). The transfected cells were then maintained for 5 days at 37° C. with 5% CO₂, then the cell supernatant was collected for characterization of the expression and secretion of the proteins encoded by the plasmids by sandwich ELISA of the supernatant using mouse anti-human IgG CH3 domain antibody (capture antibody, 100 μl/well, 1 μg/ml, MCA878G, Bio-Rad) and goat anti-human MIP-1α antibody (biotinylated detection antibody, 100 μl/well, 0.2 μg/ml, BAF270, R&D systems) (FIG. 5). The expression and secretion of the encoded immunostimulatory compounds FLT3L and GM-CSF was measured by a sandwich ELISA using mouse anti-human FLT3L antibody (capture antibody, 100 μl/well, 0.5 μg/ml, MAB608, R&D systems) and mouse anti human FLT3L antibody (biotinylated detection antibody, 100 μl/well, 0.1 μg/ml, BAF308, R&D Systems) (FIG. 6), and rat anti-mouse GM-CSF (capture antibody, 100 μl/well, 1.0 μg/ml mouse GM-CSF antibody, MAB415, R&D Systems) and goat anti-mouse GM-CSF (biotinylated detection antibody, 100 μl/well, 0.2 μg/ml, BAM215, R&D Systems) (FIG. 7), respectively. The expression and secretion of the encoded immunostimulatory compound CCL5 was measured by a sandwich ELISA using rat anti-mouse CCL5 (capture antibody, 100 μl/well, 1.0 μg/ml, MAB4781, R&D Systems) and goat anti-mouse CCL5 (biotinylated detection antibody, 100 μl/well, 0.2 μg/ml, BAF478, R&D Systems) (FIG. 8).

The results presented in FIG. 5 demonstrate that first polypeptide/dimeric protein, comprising a targeting unit, a dimerization unit, and an antigenic unit, encoded in VB4168, VB4169, and VB4170 was expressed and secreted from transfected HEK293 cells at similar levels as the comparison VB4194. FLT3L, encoded in VB4168, VB4169, and VB4170 as the second protein, is expressed and secreted at high levels from all 3 DNA plasmids, as shown in FIG. 6. Moreover, GM-CSF, encoded as the third protein in VB4169 and VB4170, is also expressed and secreted at high levels as shown in FIG. 7. CCL5, encoded as the fourth protein in VB4170, is expressed and secreted at high levels as shown in FIG. 8.

### Example 2

### Assessment of Immunogenicity of the DNA Plasmids VB4194. VB4168 and VB4169

The immunogenicity of the DNA plasmids VB4194 (comparison), VB4168 and VB4169 was determined by way of measuring the T cell immune response elicited in mice to which such plasmids were administered. A negative control, VB1026 encoding the polypeptide with amino acid sequence of 1-237 of SEQ ID NO: 1, was included. This DNA plasmid is identical to VB4194, but comprises neither the unit linker, nor the antigenic unit.

For all experiments with mice, the following study design was applied: Female, 6-week-old mice were obtained from Janvier Labs (France). All animals were housed in the animal facility at the Radium Hospital (Oslo, Norway). All animal protocols were approved by the Norwegian Food Safety Authority (Oslo, Norway). 5 mice/group were used for the testing of the constructs comprising an antigenic unit, whereas 3 mice/group were used for the negative control.

6 μg of the DNA plasmid was administered to BALB/c mice intramuscularly once, followed by electroporation. The spleens were collected 10 days after administration and mashed in cell strainer to obtain a single cell suspension. For each plasmid tested, a portion of the single cell suspension was used to deplete CD4+ T cells using Dynabeads™ anti-CD4 beads. Total splenocytes and CD4+depleted splenocytes were then tested for production of INF-y and TNF-α in a FluoroSpot assay according to the manufactures protocol (Mabtech).

Peptides (Table 7 below) with the same sequences as the 8 epitopes comprised in VB4194, VB4168 and VB4169 shown in Table 6 were used to re-stimulate the splenocytes harvested from mice to which these plasmids were administered:

TABLE 7

| | | | SEQ |
|---|---|---|---|
| Epitope | Gene | Sequence | ID NO: |
| C-pepM1 | E2f8 | VILPQAPSGPSYAT | 154 |
| | | YLQPAQAQMLTPP | |
| C-pepM8 | Dhx35 | EVIQTSKYYMRDVI | 155 |
| | | AIESAWLLELAPH | |
| C-pepM43 | Mtch1 | KSWIHCWKYLSVQS | 156 |
| | | QLFRGSSLLFRRV | |
| C-pepM69 | Klhl28 | GDVKIHAHKVVLAN | 157 |
| | | ISPYFKAMFTGNL | |
| C-pepM149 | 3110057O12Rik | FVSPMAHYVPGIMA | 158 |
| | | IESVVARFQFIVP | |
| C-pepM173 | Top3a | KIYEFDYHLYGQNI | 159 |
| | | TMIMTSVSGHLLA | |
| C-pepM174 | Cltc | NNLQKYIEIYVQKI | 160 |
| | | NPSRLPVVIGGLL | |
| C-pepM175 | Mms22I | TPLRKHTVHAIRKF | 161 |
| | | YLEFKGSSPPPRL | |

DNA plasmids VB4194, VB4168 and VB4169 were compared for their ability to elicit T cell immune responses against the peptides in Table 7. VB1026 was included as a negative control.

As shown in FIGS. 9-14, mice administered with the negative control VB1026 showed low basal immunogenicity against the peptides in Table 7.

VB4194 induced T cell responses against all 8 epitopes. VB4168, encoding the same first polypeptide as VB4194 and, in addition, FLT3L, induced stronger T cell responses than VB4194 (FIGS. 9-11). The co-expression of two immunostimulatory compounds, FLT3L and GM-CSF, in addition to the expression of the first polypeptide comprising the 8 epitopes, as encoded by VB4169, induced an even stronger immune response compared to VB4194 or VB4168 (FIGS. 9-11). The number of T cells secreting IFN-γ only (FIG. 9), TNF-α only (FIG. 10), and the number of INF-γ+TNF-α co-secreting cells (FIG. 11) all increased from VB4194 to VB4168 and from VB4168 to VB4169. Similarly, the number of CD8+ T cells (CD4+ T cell depleted samples) secreting IFN-γ only (FIG. 12), TNF-α only (FIG. 13), and the number of IFN-γ+TNF-α co-secreting cells (FIG. 14) all increased from VB4194 to VB4168, and from VB4168 to VB4169.

These results indicate that DNA plasmids according to the invention encoding a first polypeptide and one or more immunostimulatory compounds which are co-expressed from the plasmid as separate molecules can boost the antigen-specific immune responses against the antigens comprised in the first polypeptide compared to a DNA plasmid which only encodes said first polypeptide.

### Example 3

DNA plasmid VB4202 was designed and produced, comprising nucleic acid sequences encoding the elements/units listed in Table 4 and comprising further nucleic acid sequences encoding the elements listed in Table 8 below:

TABLE 8

| | VB4202 |
|---|---|
| Elements/Units | SEQ ID NO: 18 |
| Antigenic unit | See Table 6 |
| Co-expression | T2A |
| element | SEQ ID NO: 9 |
| Signal peptide | Natural leader sequence for mouse GM-CSF |
| | SEQ ID NO: 12 |
| Immunostimulatory | Mouse GM-CSF |
| compound | SEQ ID NO: 13 |

### Assessment of Expression and Secretion of the Proteins Encoded by VB4202

HEK293 cells (ATCC) were transiently transfected with the above-mentioned DNA plasmid as described in Example 1. The secreted first polypeptide/dimeric protein was characterized in a sandwich ELISA of the supernatant using mouse anti-human IgG CH3 domain antibody (capture antibody, 100 μl/well, 1 μg/ml, MCA878G, Bio-Rad) and goat anti-human MIP-1α antibody (biotinylated detection antibody, 100 μl/well, 0.2 μg/ml, BAF270, R&D systems) (FIG. 15). The secretion of the encoded immunostimulatory compound GM-CSF was measured in supernatant diluted 1:1000 by a sandwich ELISA using rat anti-mouse GM-CSF (capture antibody, 100 μl/well, 1.0 μg/ml mouse GM-CSF antibody, MAB415, R&D Systems) and goat anti-mouse GM-CSF (biotinylated detection antibody, 100 μl/well, 0.2 μg/ml, BAM215, R&D Systems) (FIG. 16).

The results presented in FIG. 15 demonstrate that first polypeptide/dimeric protein, comprising a targeting unit, a dimerization unit, and an antigenic unit, encoded in VB4202 was expressed and secreted from transfected HEK293 cells. GM-CSF, encoded as the second protein in VB4202 was expressed and secreted at high levels as shown in FIG. 16.

### Assessment of Immunogenicity of the DNA Plasmids VB4194 and VB4202

The immunogenicity of the DNA plasmids VB4194 (comparison), VB1026 (negative control) and VB4202 was determined in BALB/c mice as described in Example 2.

As shown in FIG. 17, no IFN-γ production was detected in response to administration with VB1026. VB4194 induced T cell responses against all 8 epitopes. VB4202, encoding the same first polypeptide as VB4194 and, in addition, GM-CSF, induced even stronger T cell responses than VB4194 analyzed with IFN-γ FluoroSpot.

### Flow Cytometry Assessment

Multi flow cytometry was used to evaluate APC/dendritic cell influx on a single cell level in mice administered with VB1026, VB4194 and VB4202. Female, 6-week-old BALB/c mice were obtained from Janvier Labs (France). All animals were housed in the animal facility at the Oslo University. All animal protocols were approved by the Norwegian Food Safety Authority (Oslo, Norway). 6 mice per group were used to compare VB1026, VB4202 and VB4194. A group of 6 mice that were not treated were used as a further control. 6 μg of each DNA plasmid was administered intramuscularly in the Tibialis anterior muscle, followed by electroporation. The untreated group did not receive either a DNA plasmid or the electroporation. Tibialis anterior muscles were extracted under sterile conditions, 1, 2 or 4 days after the administration or in the untreated group. To obtain single cell suspensions, the muscles were first mechanically dissociated using scissors, and then enzymatically digested. For enzymatic digestion, the dissociated muscles were incubated in digestion medium (DMEM, Collagenase A [2 mg/ml], DNase [50 U/ml]) for 1 h, with stirring magnet, at 37° C. Following the incubation, the single cell suspension was filtered through a 70 μm filter and washed twice at 400×g for 6 min at 4° C. in PBS.

For flow cytometry analysis, the single cell suspension was first incubated with viability dye (eFluor 780, Invitrogen) at room temperature (RT) for 10 min. The viability dye was rinsed off with PBS (centrifuged twice at 400×g for 6 min at 4° C.). Cells were then incubated with Fc block for 10 min at RT, to block unspecific binding of fluorescent antibodies. Following the blocking step, cells were stained with a pool of surface marker specific antibodies (Table 9 below) for 30 minutes on ice. The stained cells were run on the BD FACSymphony A5 Flow cytometer. Flow cytometry data were analyzed using FlowJo software. A gating strategy was used to define dendritic cells (DCs)/APCs as described in the description of FIG. 18.

TABLE 9

| Marker | Fluorochrome | Function |
|---|---|---|
| CD45 | BUV661 | All immune cells |
| B220 | BUV496 | Defining B cells/pDCs |
| CD317 (BST2) | BUV737 | Defining pDCs |
| I-A/I-E | Brilliant Violet 711 | MHC II-all antigen presenting |
| | | cells including all DCs |
| XCR1 | Brilliant Violet 650 | Defining cDC1 |
| CD172a | PerCP-eFluor 710 | Defining cDC2 |
| (SIRP alpha) | | |
| CD64 | BUV805 | Defining monocytes and mDCs |
| CD24 | BUV615 | All DCs |
| Ly-6G | APC/Cy7 | Exclusion of neutrophils |
| CD3 | APC/Cy7 | Exclusion of T cells |
| Viability dye | eFluor780 | Exclusion of dead cells |

The results show an increased influx of immune cells (CD45+ cells) into the muscle (FIG. 19) of mice to which VB4202 was administered compared to muscle of mice to which VB4194 was administered.

The proportion of DCs within the CD45+ cell population present in the muscle was higher in mice that received VB4202 compared to mince that received VB4194 (FIG. 20). Moreover, the cDC1 population (FIG. 21) and the moDC population (FIG. 22) were both increased in the muscle of mice that received VB4202 compared to those that received VB4194.

In summary, these results indicate that DNA plasmids according to the invention encoding a first polypeptide and one or more immunostimulatory compounds which are co-expressed from the plasmid as separate molecules can boost the influx of dendritic cells to the location of administration, when administered intramuscularly, ultimately further contributing to an increased antigen-specific immune response against the antigens comprised in the first polypeptide compared to a DNA plasmid which only encodes said first polypeptide.

### Example 4

The following DNA plasmids were designed and produced:

All DNA plasmids, VB1020, VB4195, VB4196, comprise nucleic acid sequences encoding the elements/units listed in Table 4 and further comprise nucleic acid sequences encoding the elements/units listed in Table 10 below:

TABLE 10

| | VB1020 | VB4195 | VB4196 |
|---|---|---|---|
| Element/Units | SEQ ID NO: 19 | SEQ ID NO: 20 | SEQ ID NO: 21 |
| Antigenic unit | HPV16 E7 antigen and HPV16 E6 antigen separated | | |
| | by a glycine-serine linker | | |
| Co-expression | NA | T2A SEQ ID NO: 9 | |
| element | | | |
| Signal peptide | NA | Natural leader sequence for human | |
| | | FLT3L SEQ ID NO: 4 | |
| Immunostimulatory | NA | Human FLT3L | |
| compound 1 | | SEQ ID NO: 10 | |
| Co-expression | NA | NA | P2A |
| element | | | SEQ ID NO: 11 |
| Signal peptide | NA | NA | Natural leader sequence |
| | | | for mouse GM-CSF |
| | | | SEQ ID NO: 12 |
| Immunostimulatory | NA | NA | Mouse GM-CSF |
| compound 2 | | | SEQ ID NO: 13 |

DNA plasmids VB1020, VB4195 and VB4196 comprise nucleic acid sequences encoding for a first polypeptide comprising an antigenic unit comprising human papilloma virus 16 (HPV16) antigens E7 and E6.

Each of these DNA plasmids is a model of a DNA plasmid according to the invention encoding for a non-individualized first polypeptide for use in the treatment of cancer, i.e. one that comprises an antigenic unit comprising several shared cancer antigens, with the shared cancer antigens being viral shared cancer antigens (here: antigens from HPV16 which is responsible for certain types of cancer) or a model of a DNA plasmid according to the invention encoding a first polypeptide for use in the treatment of infectious diseases, i.e. one that comprises an antigenic unit comprising antigens derived from a pathogen (here: antigens derived from HPV16).

The DNA plasmids VB4195 and VB4196 allow the co-expression of a first polypeptide as described above and the following immunostimulatory compound(s), as separate molecules:
VB1020: encodes only a first polypeptide, no immunostimulatory compound and serves as a comparison.
VB4195: hFLT3L.
VB4169: hFLTL3 and mGM-CSF

### Assessment of Expression and Secretion of the Proteins Encoded by DNA Plasmids

HEK293 cells were obtained from ATCC and transiently transfected with VB1020 (comparison), VB4195 or VB4196 as described in Example 1.

The secreted proteins encoded by the DNA plasmids were characterized in a sandwich ELISA of the supernatant using mouse anti-human IgG CH3 domain antibody (capture antibody, 100 μl/well, 1 μg/ml, MCA878G, Bio-Rad) and goat anti-human MIP-1a antibody (biotinylated detection antibody, 100 μl/well, 0.2 μg/ml, R&D systems, BAF270).

The secretion of FLT3L, encoded as the second protein in VB4195 and VB4196, in cell culture supernatant (diluted 1:500) was measured by a sandwich ELISA using mouse anti-human FLT3L antibody (capture antibody, 100 μl/well, 0.5 μg/ml, MAB608, R&D systems) and mouse anti human FLT3L antibody (biotinylated detection antibody, 100 μl/well, 0.1 μg/ml, BAF308, R&D Systems). The secretion of GM-CSF in cell culture supernatant (diluted 1:500), encoded as the third protein in VB4196, was measured by a sandwich ELISA using rat anti-mouse GM-CSF (capture antibody, 100 μl/well, 1.0 μg/ml mouse GM-CSF antibody, MAB415, R&D Systems) and goat anti-mouse GM-CSF (biotinylated detection antibody, 100 μl/well, 0.2 μg/ml, BAM215, R&D Systems).

The results presented in FIG. 23 demonstrate that a first polypeptide/dimeric protein comprising a targeting unit, a dimerization unit, and an antigenic unit, encoded in VB4195 and VB4196 was well expressed and secreted from transfected HEK293 cells. FLT3L, encoded in VB4195 and VB4196 as the second protein, is expressed and secreted at high levels from both plasmids, as shown in FIG. 24. Moreover, GM-CSF, encoded as the third protein in VB4196, is also expressed and secreted at high levels as shown in FIG. 25.

Characterization of the Intact Proteins Expressed from VB4195 and VB4196

Western blot analysis was performed on supernatant samples from transfected Expi293F cells to further characterize the proteins encoded by VB4195 and VB4196. VB1020, encoding an identical first polypeptide as VB4195 and VB4196, was included as a comparison.

Briefly, Expi293F cells (3×10⁶ cells/ml, 1.6 ml) were seeded in a 6-well culture plate. The cells were transfected with 1 μg/ml plasmid DNA using ExpiFectamine 293 Reagent (Thermo Fisher Sci.), and the plates were incubated on an orbital shaker (19 mm diameter, 125 rpm) in a humidified CO₂ cell incubator (8% CO₂, 37° C.). After 18 h of incubation, ExpiFectamine 293 Transfection Enhancer (Thermo Fisher Sci.) was added to each well. The plates were incubated for another 28 h before the supernatant was harvested. The samples were prepared by mixing 70 μl supernatant from transfected Expi293F cells with 25 μl 4× Laemmli sample buffer (Bio-Rad) with 5 μl DTT (Thermo Fisher Sci.) or 5 μl ultrapure water for reducing and non-reducing conditions, respectively. Moreover, Expi293F supernatants were deglycosylated by mixing 64 μl sample with 16 μl PNGase F buffer (NEB) and incubated at 80° C. for 2 min. After cooling down, 4 μl Rapid PNGase F enzyme (NEB) was added, and the samples were incubated at 50° C. for 10 min. The deglycosylated samples were further mixed with 30 μl 4× Laemmli buffer and 6 μl DTT. The samples (reduced, non-reduced, or deglycosylated) were heated at 70° C. for 10 minutes and added to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with goat anti-human MIP-1a (BAF270, R&D Systems), goat anti-murine GM-CSF (BAF415, R&D Systems), or goat anti-human FLT3L (BAF308, R&D Systems) to detect the first polypeptide/dimeric protein, GM-CSF, or FLT3L, respectively. The specificity of the primary antibodies was confirmed in an initial test probing their respective recombinant proteins. The membranes were incubated with fluorochrome-conjugated secondary antibodies for 1 h at RT, and then washed and dried. Images were acquired by using a ChemiDoc™ MP Imaging System (setting Dylight 550 and 650, Auto Optimal).

The western blot analysis confirmed the ELISA results demonstrating that VB4195 expressed two proteins: a first polypeptide/dimeric protein (FIG. 26); and FLT3L (FIG. 27). VB4196 expressed three proteins: a first polypeptide/dimeric protein (FIG. 26), FLT3L and GM-CSF (FIG. 27). The P2A sequence used to separate nucleic acid sequences encoding the FLT3L and GM-CSF proteins in VB4196 appeared to be glycosylated, creating a shift in the protein size observed in the western blot. The deglycosylation protocol with PNGase F reduced this size shift. Moreover, the P2A peptide leaves a 21 amino acid tail attached to the C-terminal end of FLT3L, which can be observed in the western blot by a resulting size shift of approximately 2.2 kDa. Importantly, no additional bands were observed for the anti-FLT3L and anti-GM-CSF probed membranes, demonstrating successful ribosome skipping at the P2A and T2A sequences, resulting in expression of multiple, separate proteins from a single DNA plasmid.

Taken together, the ELISA and western blot data demonstrate that intact dimeric proteins, comprising a targeting unit, dimerization unit and antigenic unit, can be co-expressed from a DNA plasmid together with one or more other proteins (immunostimulatory compounds) by using as co-expression elements different 2A peptides.

### Example 5

DNA plasmid VB4204 was designed and produced, comprising nucleic acid sequences encoding the elements/units listed in Table 4 and further comprising nucleic acid sequences encoding the elements/units listed in Table 11 below:

TABLE 11

| | VB4204 |
|---|---|
| Element/Units | SEQ ID NO: 22 |
| Antigenic unit | HPV16 E7 antigen and HPV16 E6 antigen |
| | separated by a glycine-serine linker |
| Co-expression element | T2A |
| | SEQ ID NO: 9 |
| Signal peptide | Natural leader sequence for mouse GM-CSF |
| | SEQ ID NO: 12 |
| Immunostimulatory | Mouse GM-CSF |
| compound | SEQ ID NO: 13 |

Further, DNA plasmid pGM-CSF was designed and produced by cloning the sequences of the natural leader sequence for mouse GM-CSF (SEQ ID NO: 12) and mouse GM-CSF (SEQ ID NO: 13) into the expression vector pUMVC4a.

### Assessment of Expression and Secretion of the Proteins Encoded by VB4204

HEK293 cells were obtained from ATCC and transiently transfected with VB4204 or VB1020 (comparison) as described in Example 1.

The secreted proteins encoded by VB4204 or VB1020 were characterized in a sandwich ELISA of the supernatant (diluted 1:10) using mouse anti-human IgG CH3 domain antibody (capture antibody, 100 μl/well, 1 μg/ml, MCA878G, Bio-Rad) and goat anti-human MIP-1α antibody (biotinylated detection antibody, 100 μl/well, 0.2 μg/ml (R&D systems, BAF270).

The secretion of GM-CSF, encoded as the second protein in VB4204, was measured by a sandwich ELISA on cell culture supernatant (diluted 1:1000) using rat anti-mouse GM-CSF (capture antibody, 100 μl/well, 1.0 μg/ml mouse GM-CSF antibody, MAB415, R&D Systems) and goat anti-mouse GM-CSF (biotinylated detection antibody, 100 μl/well, 0.2 μg/ml, BAM215, R&D Systems).

The results presented in FIG. 28 demonstrate that the first polypeptide/dimeric protein, comprising a targeting unit, a dimerization unit and an antigenic unit, encoded in VB4204 is well expressed and secreted from transfected HEK293 cells. Moreover, GM-CSF, encoded in VB4204 as a separate, second protein, is also expressed and secreted at high levels as shown in FIG. 29.

### Assessment of Immunogenicity of VB4204 (1)

Immunogenicity of VB4204, VB1020 (comparison) and VB1026 (negative control) was determined as described in Example 2 in C57BL/6 mice, however, no CD4+ T cell depleted splenocytes data were generated. The T cell responses in the splenocytes were then tested for production of INF-y in a FluoroSpot assay. Further, immunogenicity of co-injected DNA plasmids (6 μg total DNA) VB1020 (encoding the same polypeptide as VB4204, but does not encode GM-CSF) and pGM-CSF (encoding GM-CSF but does not encode the polypeptide of VB4204) was determined as described in this paragraph.

Peptides corresponding to the E6 and E7 antigens described in Table 12 below were used to re-stimulate the splenocytes harvested from mice administered with VB1020, VB4204, VB1026 and (VB1020 plus pGM-CSF).

TABLE 12

| Re- | | | SEQ |
|---|---|---|---|
| stimulation | Gene | Sequence | ID NO: |
| Single | HPV16 E7 | RAHYNIVTF | 162 |
| peptide | (49-57) | | |
| Peptide | HPV16 E6 | MFQDPQERPRKLPQL | 163 |
| pool | | RPRKLPQLCTELQTT | 164 |
| | | LCTELQTTIHDIILE | 165 |
| | | TIHDIILECVYCKQQ | 166 |
| | | ECVYCKQQLLRREVY | 167 |
| | | QLLRREVYDFARRDL | 168 |
| | | YDFARRDLCIVYRDG | 169 |
| | | LCIVYRDGNPYAVRD | 170 |
| | | GNPYAVRDKCLKFYS | 171 |
| | | DKCLKFYSKISEYRH | 172 |

VB1020 (first polypeptide only), VB4204 (first polypeptide and GM-CSF) and a co-injection of VB1020 plus pGM-CSF were compared for their ability to elicit T cell immune response against the peptides in Table 12. VB1026 was included as a negative control.

As shown in FIG. 30, no IFN-γ production was detected in response to administration with VB1026.

Moreover, VB1020 induced strong T cell responses against the peptides in Table 12, while VB4202 induced even stronger T cell responses compared to VB1020. Furthermore, VB4202 also induced stronger T cell responses than induced by the co-injection of VB1020 plus pGM-CSF (FIG. 30).

### Assessment of Immunogenicity of VB4204 (2)

Immunogenicity of VB4204, VB1020 (comparison) and VB1026 (negative control) was determined by flow cytometry. C57BL/6 mice were treated as described in Example 2, splenocytes were pooled group wise and re-stimulated with the single peptide corresponding to the HPV16 E7 (49-57) antigen as described in Table 12 for 1 h at RT before monensin and brefeldin were added to each well to inhibit endocytosis. The cells were further incubated for 15 h at 37° C. Following re-stimulation, the cells were harvested for flow cytometry analysis. Briefly, the single cell suspension was first incubated with viability dye (eFluor 780, Invitrogen) at RT for 10 min. The viability dye was rinsed off with PBS (centrifuged twice at 400×g for 6 min at 4° C.). Cells were then incubated with Fc block for 10 min at RT, to block unspecific binding of fluorescent antibodies. Following the blocking step, cells were stained with a pool of surface marker specific antibodies (Table 9) for 30 minutes on ice. The antibodies were rinsed off with PBS (centrifuged twice at 400×g for 6 min at 4° C.) and the cells were incubated with fixation/permeabilization solution (60 min at 4° C.). The cells were centrifuged and washed and re-suspended in 100 μl antibody mix in permeabilization buffer and incubated for 30 min at 4° C. The stained cells were run on the BD FACSymphony A5 Flow cytometer. Flow cytometry data were analyzed using FlowJo software.

DNA plasmid VB4202 was compared for its ability to elicit T cell immune responses against the single peptide HPV16 E7 (49-57) in Table 12. VB1020 was included as a comparison; VB1026 was included as a negative control.

As shown in FIG. 31, no IFN-γ or TNF-α production was detected in response to administration with VB1026.

The number of CD8+ T cells (CD4+ T cell depleted samples) secreting IFN-γ only, TNF-α only, and the number of INF-γ+TNF-α co-secreting cells were all increased from VB1020 to VB4202 (FIG. 31).

These results indicate that a DNA plasmid according to the invention encoding a first polypeptide and an immunostimulatory compound which are co-expressed from the plasmid as a separate molecule can boost the antigen-specific T cell responses against the antigens comprised in the first polypeptide, compared to a DNA plasmid which only encodes said first polypeptide and compared to a co-injection of a DNA plasmid encoding the same first polypeptide and a plasmid encoding the same immunostimulatory compound.

### Example 6

DNA plasmid VB4205 was designed and produced, comprising nucleic acid sequences encoding the elements/units listed in Table 4 and further comprising nucleic acid sequences encoding the elements/units listed in Table 13 below:

TABLE 13

| | VB4205 |
|---|---|
| Elements/Units | SEQ ID NO: 23 |
| Antigenic unit | HPV16 E7 antigen and HPV16 E6 antigen separated |
| | by a glycine-serine linker |
| Co-expression | T2A |
| element | SEQ ID NO: 9 |
| Signal peptide | Natural leader sequence for mouse CCL5 |
| | SEQ ID NO: 15 |
| Immunostimulatory | Mouse CCL5 |
| compound | SEQ ID NO: 16 |

### Assessment of Expression and Secretion of Proteins Encoded by VB4205

HEK293 cells were obtained from ATCC and transiently transfected with VB4205 or VB1020 (comparison) as described in Example 1. The secreted proteins encoded by VB4205 or VB1020 were characterized in a sandwich ELISA of the supernatant (diluted 1:10) using mouse anti-human IgG CH3 domain antibody (capture antibody, 100 μl/well, 1 μg/ml, MCA878G, Bio-Rad) and goat anti-human MIP-1α antibody (biotinylated detection antibody, 100 μl/well, 0.2 μg/ml (R&D systems, BAF270).

The secretion of CCL5, encoded as the second protein in VB4205, was measured in supernatant (diluted 1:1000) by a sandwich ELISA using rat anti-mouse CCL5 (capture antibody, 100 μl/well, 1.0 μg/ml, MAB4781, R&D Systems) and goat anti-mouse CCL5 (biotinylated detection antibody, 100 μl/well, 0.2 μg/ml, BAF478, R&D Systems).

The results presented in FIG. 32 demonstrate that the first polypeptide/dimeric protein, comprising a targeting unit, a dimerization unit and an antigenic unit, encoded in VB4205 is well expressed and secreted from transfected HEK293 cells. Moreover, CCL5 encoded in VB4205 as the second protein is expressed and secreted at high levels as shown in FIG. 33.

### Assessment of Immunogenicity of VB4205

Immunogenicity of VB4205 was determined in C57BL/6 mince and compared to immunogenicity of VB1020 (comparison) and VB1026 (negative control) as described in Example 5 (1).

VB1020 (first polypeptide only) and VB4205 (first polypeptide and CCL5) were compared for their ability to elicit T cell immune response against the peptides in Table 12.

As shown in FIG. 34, no IFN-γ production was detected in response to administration with VB1026. VB1020 induced strong T cell responses against the peptides in Table 12, while VB4205 induced even stronger T cell responses compared to VB1020.

Also these results indicate a DNA plasmid according to the invention encoding a first polypeptide and an immunostimulatory compound which are co-expressed from the plasmid as a separate molecule can boost the antigen-specific T cell responses against the antigens comprised in the first polypeptide, compared to a DNA plasmid which only encodes said first polypeptide.

### Example 7

DNA plasmids VB1026, VB4208, VB4194, VB4202 and pGM-CSF were designed and produced as described herein, comprising nucleic acid sequences encoding the elements/units listed in Table 14:

TABLE 14

| Elements/ | | | | VB4202 | |
|---|---|---|---|---|---|
| Units | VB1026 | VB4208 | VB4194 | (SEQ ID NO: 18) | pGM-CSF |
| Signal | Table 4 | Table 4 | Table 4 | Table 4 | NA |
| peptide | | | | | |
| Targeting | Table 4 | Table 4 | Table 4 | Table 4 | NA |
| unit | | | | | |
| Dimerization | Table 4 | Table 4 | Table 4 | Table 4 | NA |
| unit | | | | | |
| Unit linker | NA | Table 4 | Table 4 | Table 4 | NA |
| Antigenic | NA | NA | Table 6 | Table 6 | NA |
| unit | | | | | |
| Co- | NA | T2A | NA | T2A | NA |
| expression | | SEQ ID NO: 9 | | SEQ ID NO: 9 | |
| element | | | | | |
| Signal | NA | Natural leader | NA | Natural leader sequence for | |
| peptide | | sequence for mouse | | mouse GM-CSF | |
| | | GM-CSF | | SEQ ID NO: 12 | |
| | | SEQ ID NO: 12 | | | |
| Immuno- | NA | Mouse GM-CSF | NA | Mouse GM-CSF | |
| stimulatory | | SEQ ID NO: 13 | | SEQ ID NO: 13 | |
| compound | | | | | |

The DNA plasmids encode the following proteins:
VB4202 encodes and allows for the co-expression of a first polypeptide as described above and the immunostimulatory compound mGM-CSF as separate molecules
VB4194: encodes only a first polypeptide comprising an antigenic unit comprising CT26 epitopes, no immunostimulatory compound and serves as a comparison.
VB1026: encodes the polypeptide with amino acid sequence of 1-237 of SEQ ID NO: 1, which is identical to the first polypeptide encoded by VB4194, but neither comprises the unit linker, nor the antigenic unit. It serves as a negative control
VB4208 (SEQ ID NO: 24): encodes a first polypeptide which does not comprise an antigenic unit, i.e. does not encode any CT26 epitopes, and mGM-CSF as separate molecules. It serves as a negative control
pGM-CSF: encodes mGM-CSF and serves as a comparison

### Treatment of CT26-Tumor Challenged Mice

The antitumor efficacy of VB4202 was assessed in a CT26 tumor challenge. VB4202 was compared to VB4194 encoding the same first polypeptide as VB4202. Furthermore, the antitumor efficacy of VB4202 was compared to co-injections of VB4194 with pGM-CSF. VB1026 and VB4208 were included as negative controls.

Each of the groups A-F contained 8 BALB/c mice, which were inoculated with CT26 tumor cells on day (D) 0 by injection of 1×10⁵ tumor cells in the left leg. On days 4 and 11, the DNA plasmids and their respective amounts described in Table 15, were administered intramuscularly to the right leg of the mice. Due to plasmid size variations between the VB4194 and pGM-CSF plasmids, a second co-injection group (group F) was included where the amount of each plasmid was adjusted to match the plasmid copy number for the single plasmid injection in group D (Table 16) to ensure that comparable protein levels are expressed.

TABLE 15

| Treatment group | DNA plasmid and amount |
|---|---|
| A | VB1026, 10 μg |
| B | VB4208, 10 μg |
| C | VB4194, 10 μg |
| D | VB4202, 10 μg |
| E: Co-injection | VB4194, 5 μg and |
| | pGM-CSF, 5 μg |
| F: Co-injection | VB4194, 9.3 μg and |
| Copy number adjusted | pGM-CSF, 6.4 μg |

TABLE 16

| DNA | Plasmid size | DNA copy number/ | Factor to |
|---|---|---|---|
| plasmid | [bp] | μg DNA plasmid | VB4202 |
| pGM-CSF | 4194 | 2.32 × 10¹¹ | 0.64 |
| VB4194 | 6126 | 1.59 × 10¹¹ | 0.93 |
| VB4202 | 6561 | 1.49 × 10¹¹ | 1 |

Tumor size was measured using a caliper. The tumors were measured in two dimensions, length and width, and the height was set equal to the width. The tumor volume was calculated by the formula: Tumor vol.=Length (mm)×width (mm)×height (mm)/2000. The treatment was concluded on day 32.

The tumors in the groups that had been treated with VB4194 (group C), VB4202 (group D) and with a co-injection of VB4194 and pGM-CSF (groups E and F) grew slower compared to the VB1026 and VB4208 negative control groups (groups A and B, respectively).

Administration of VB4202, co-expressing the same first polypeptide as VB4194 and GM-CSF (group D), resulted in a reduced tumor growth rate compared to VB4194 alone (group C). Moreover, administration of VB4202 resulted in reduced tumor growth rate compared to the two co-injection groups were the VB4194 and pGM-CSF was administered at a total of 10 μg (E) or adjusted to comparable copy numbers (F). Such co-injections resulted in a similar tumor growth rate than the administration of VB4194 alone.

These results (shown in FIG. 35) show that the tumor growth inhibition efficacy provided by VB4194 was further increased by the co-expression of GM-CSF from VB4202, which encodes for the same first polypeptide as VB4194. The tumor growth inhibition was accompanied by increased survival rate in the VB4202 treated animals compared to the other groups as shown in FIG. 36. The antitumor efficacy was driven by antigen specific immune responses, as shown by comparing VB4202 with the negative controls VB1026 and VB4208. Moreover, VB4202 provided a stronger antitumor efficacy than that observed when co-injecting VB4194 with pGM-CSF as two separate plasmids.

### Example 8

DNA plasmids TECH001-CV021, TECH001-CV022 and TECH001-CV023 were designed and produced, comprising nucleic acid sequences encoding the elements/units listed in Table 4 and further comprising nucleic acid sequences encoding the elements/units listed in Table 17 below:

TABLE 17

| | | TECH001-CV021 | TECH001-CV022 | TECH001-CV023 |
|---|---|---|---|---|
| Elements/Units | VB2060 | SEQ ID NO: 31 | SEQ ID NO: 32 | SEQ ID NO: 33 |
| Antigenic unit | SARS-COV-2 receptor-binding domain (RBD) | | | |
| | SEQ ID NO: 30 | | | |
| Co-expression | NA | T2A | T2A | T2A |
| element | | SEQ ID NO: 9 | SEQ ID NO: 9 | SEQ ID NO: 9 |
| Signal peptide | NA | Natural leader | Natural leader | Natural leader |
| | | sequence for mouse | sequence for mouse | sequence for mouse |
| | | GM-CSF | IL-12a | IL-21 |
| | | SEQ ID NO: 12 | SEQ ID NO: 34 | SEQ ID NO: 38 |
| Immunostimulatory | NA | Mouse GM-CSF | Mouse IL-12A | Mouse IL-21 |
| compound 1A | | SEQ ID NO: 13 | SEQ ID NO: 35 | SEQ ID NO: 39 |
| Co-expression | NA | NA | T2A | NA |
| element | | | SEQ ID NO: 9 | |
| Signal peptide | NA | NA | Natural leader | NA |
| | | | sequence for mouse | |
| | | | IL-12b | |
| | | | SEQ ID NO: 36 | |
| Immunostimulatory | NA | NA | Mouse IL-12B | NA |
| compound 1B | | | SEQ ID NO: 37 | |

DNA plasmids TECH001-CV021, TECH001-CV022 and TECH001-CV023 comprise nucleic acid sequences encoding for a first polypeptide comprising an antigenic unit comprising the SARS-CoV-2 receptor-binding domain (RBD) antigen.

Each of these DNA plasmids is a model of a DNA plasmid according to the invention encoding for a first polypeptide for use in the treatment of infectious diseases, i.e. one that comprises an antigenic unit comprising antigens derived from a pathogen (here: antigens derived from SARS-CoV-2).

The DNA plasmids TECH001-CV021, TECH001-CV022 and TECH001-CV023 allow the co-expression of a first polypeptide as described above and the following immunostimulatory compound(s), as separate molecules:
VB2060: encodes only a first polypeptide, no immunostimulatory compound and serves as a comparison
TECH001-CV021: mGM-CSF
TECH001-CV022: mIL-12
TECH001-CV023: mIL-21

IL 12 is a heterodimeric cytokine encoded by two separate genes, IL-12A (p35) and IL-12B (p40). The active heterodimer (referred to as p70), and a homodimer of p40 are formed following protein synthesis.

### Assessment of Expression and Secretion of Proteins Encoded by DNA Plasmids

Briefly, Expi293F cells (2×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). The plates were incubated for 72 h before the supernatant was harvested.

The secreted first polypeptides/dimeric proteins in the supernatants (diluted 1:1500) were characterized in a sandwich ELISA using mouse anti-human IgG CH3 domain antibody (capture antibody, 100 μl/well, 1 μg/ml, MCA878G, Bio-Rad) and goat anti-human MIP-1α antibody (biotinylated detection antibody, 100 μl/well, 0.2 μg/ml, BAF270, R&D systems) (FIG. 37).

The protein expression and secretion of immunostimulatory compounds GM-CSF, IL-12, and IL-21 in the supernatants was measured by a sandwich ELISA (FIGS. 38a, 38b, and 38c, respectively) using anti-mouse GM-CSF Abs (rat anti-mouse GM-CSF capture antibody, 100 μl/well, 1.0 μg/ml mouse GM-CSF antibody, MAB415, R&D Systems; goat anti-mouse GM-CSF biotinylated detection antibody, 100 μl/well, 0.2 μg/ml, BAM215, R&D Systems) (FIG. 38a), anti-mouse IL-12 Abs (rat anti-mouse IL-12 capture antibody, 100 μl/well, 1.0 μg/ml, MAB419 R&D systems; goat anti-mouse IL-12 biotinylated detection antibody, 100 μl/well, 0.4 μg/ml, BAF419, R&D systems) (FIG. 38b), and anti-mouse IL-21 Abs (goat anti-mouse IL-21 capture antibody, 100 μl/well, 0.1 μg/ml, AF594, R&D systems; goat anti-mouse IL-21 biotinylated detection antibody, 100 μl/well, 0.4 μg/ml, BAF594, R&D systems) (FIG. 38c), respectively.

The results presented in FIG. 37 demonstrate that first polypeptide/dimeric protein, comprising a targeting unit, a dimerization unit, and an antigenic unit, encoded in TECH001-CV021, TECH001-CV022, and TECH001-CV023 was expressed and secreted from transfected Expi293F cells. GM-CSF, IL-12 and IL-21 encoded as the second protein in TECH001-CV021, TECH001-CV022 and TECH001-CV023, respectively, was also expressed and secreted at high levels as shown in FIGS. 38a-c.

Characterization of the Intact Proteins Expressed from TECH001-CV021, TECH001-CV022 and TECH001-CV023

Western blot (WB) analysis was performed on supernatant samples from transfected Expi293F cells to further characterize the proteins encoded by TECH001-CV021, TECH001-CV022 and TECH001-CV023. VB2060, encoding an identical first polypeptide as the aforementioned DNA plasmids, was included as a comparison.

The samples were prepared by mixing 70 μl supernatant from transfected Expi293F cells with 25 μl 4× Laemmli sample buffer (Bio-Rad) with 5 μl DTT (Thermo Fisher Sci.) or 5 μl ultrapure water for reducing and non-reducing conditions, respectively. The samples (reduced or non-reduced) were heated at 70° C. for 10 minutes before adding 20 μL per lane to 4%-20% Criterion TGX Stain-Free precast gels (Bio-Rad). SDS-PAGE was performed in 1× Tris/Glycine/SDS running buffer (Bio-Rad) with Precision Plus Protein All Blue Prestained and Unstained protein standards (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with goat anti-human MIP-1α (AF270, R&D Systems), goat anti-murine GM-CSF (BAF415, R&D Systems), goat anti-mouse IL-12 (BAF419, R&D Systems), or goat anti-mouse IL-21 (BAF594, R&D Systems) to detect the first polypeptide, GM-CSF, IL-12, and IL-21, respectively. The membranes were washed, incubated with fluorochrome-conjugated anti-goat secondary antibodies for 1 h at RT, and then washed and dried (rinsed in ethanol). Images were acquired by using a ChemiDoc™ MP Imaging System (setting Dylight 650 and 800, Auto Optimal). Expifectamine treated cells (transfection control) was included as a negative control on each gel.

The WB analysis confirmed the ELISA results, demonstrating that an intact first polypeptide was expressed from all four DNA plasmids, TECH001-CV021, TECH001-CV022, TECH001-CV023 and VB2060 (FIG. 39). TECH001-CV021 further expressed (heterogeneously glycosylated) GM-CSF (FIG. 40). FIG. 41 shows the WB analysis of TECH001-CV022 probed with goat anti-mouse IL-12 under reducing (left panel) and non-reducing (right panel) conditions. In addition to expressing the first polypeptide, TECH001-CV022 expressed glycosylated IL-12B (p40) and IL-12A (p35) (FIG. 41, left panel). Previous studies reported that cells that secrete bioactive IL-12 (p70 heterodimer) also secrete p40 (monomer) in free form (Jalah et al., J Biol Chem Vol 288, No. 9, 6763-6776, 2013). Indeed, bands for both IL-12 p70 heterodimer and p40 monomer were detected under non-reducing conditions (FIG. 41, right panel). In addition to the first polypeptide, TECH001-CV023 expressed IL-21 (FIG. 42). Importantly, no additional bands were observed for the anti-GM-CSF, anti-IL-12 and anti-IL-21 probed membranes, demonstrating successful ribosome skipping at the T2A sequences, resulting in expression of multiple, separate proteins from a single DNA plasmid.

Taken together, the ELISA and western blot data demonstrate that intact first polypeptides, comprising a targeting unit, dimerization unit and antigenic unit, can be co-expressed from a DNA plasmid together with one or more immunostimulatory compounds by using as co-expression elements identical 2A peptides.

### Assessment of Immunogenicity of TECH001-CV021, TECH001-CV022 and TECH001-CV023

Immunogenicity of TECH001-CV021, TECH001-CV022 and TECH001-CV023 was determined and compared to immunogenicity of VB2060 and VB1026 (negative control).

Female, 6-week-old BALB/c mice were obtained from Janvier Labs (France). All animals were housed in the animal facility at the University of Oslo (Oslo, Norway). All animal protocols were approved by the Norwegian Food Safety Authority (Oslo, Norway). 5 mice/group were used for the testing of TECH001-CV021, TECH001-CV022, TECH001-CV023 and VB2060, whereas 3 mice/group were used for the negative control.

A final dose of 1 μg DNA plasmid was administered by intramuscular needle injection to each tibialis anterior (2×25 μl, 20 μg/ml), followed by electroporation with AgilePulse in vivo electroporation system (BTX, USA).

### Assessment of Humoral Immune Response Induced in Mice Against SARS-CoV-2 RBD.

Sera from the mice administered with the DNA plasmids were collected 13 days after their administration and tested for anti-RBD IgG antibodies binding the RBD protein (Wuhan variant).

Briefly, blood was collected from the saphenous vein of the vaccinated mice. Coagulated blood was centrifuged twice (1000 g, 15 min) and the serum was collected and transferred to a clean tube. The humoral immune response was evaluated in an ELISA assay detecting total IgG in the sera binding to RBD (aa319-542) from SARS-CoV2 (Wuhan variant). ELISA plates (MaxiSorp Nunc-Immuno plates) were coated with 1 μg/ml recombinant RBD-His protein antigen in PBS overnight at 4° C. Plates were blocked with 4% BSA in PBS for 1 h at RT. Plates were then incubated with serial dilutions of mouse sera (diluted in 0.1% BSA in PBS) and incubated for 2 h at 37° C. Plates were washed 3× and incubated with 1:50 000 dilution of anti-mouse total IgG-HRP antibody (Southern Biotech) in 0.1% BSA in PBS and incubated for 1 h at 37° C. After final washing, plates were developed using TMB substrate (Merck, cat. CL07-1000). Plates were read at 450 nm wavelength within 30 min using a SPARK® Multimode Microplate Reader (Tecan). Binding antibody endpoint titers were calculated as the reciprocal of the highest dilution resulting in a signal above the cutoff. Binding antigens tested included SARS-CoV-2 antigens: RBD (Sino Biological 40592-V08H; (SEQ ID NO: 30)).

The results shown in FIG. 43 demonstrate that TECH001-CV021 and TECH001-CV023, encoding a first polypeptide comprising RBD (aa 319-542) from SARS-CoV-2 (Wuhan variant) in the antigenic unit and GM-CSF and IL-21, respectively, as a second protein, induced stronger total IgG responses against RBD than the comparison VB2060, encoding only the aforementioned first polypeptide (Mann-Whitney test, TECH001-CV021: P=0.008, TECH001-CV023: P=0.047). Moreover, TECH001-CV022, encoding the aforementioned first polypeptide and two sub-domains of IL-12 as a second and third protein also induced stronger IgG responses against RBD than the comparison VB2060 (Mann-Whitney test, P=0.047).

### Assessment of T Cell Responses Induced Against SARS-CoV-2 RBD

The spleens of mice administered with the DNA plasmids were collected 14 days after administration and mashed in a cell strainer to obtain a single cell suspension. The red blood cells were lysed using ammonium-chloride-potassium (ACK) lysing buffer. The splenocytes were counted using the NucleoCounter NC-202 (ChemoMetec, Denmark) and re-suspended to a final concentration of 6×10⁶ cells/ml. For each plasmid tested, a portion of the single cell suspension was used to deplete CD4+ T cells using Dynabeads™ anti-CD4 beads. Total splenocytes and CD4+ T cell depleted splenocytes were then tested for production of INF-y in a FluoroSpot assay by seeding 6×10⁵ cells/well and re-stimulating with 2 μg/ml RBD peptide pools (Table 18) for 22.5 hours. The RBD peptide pools comprised 15-mer peptides overlapping by 12 amino acids spanning regions of the RBD.

TABLE 18

| Pool ID | Composition |
|---|---|
| Pool 1 | RBD-1, 2, 3, 4, 5, 6, 7, 8, 9,10 |
| Pool 2 | RBD-11, 12, 13, 14, 15, 16, 17, 18, 19, 24 |
| Pool 3 | RBD-20, 21, 22, 23, 25, 26, 27, 28, 29, 30 |
| Pool 4 | RBD-31, 32, 33, 34, 36, 37, 38, 39, 40 |
| Pool 5 | RBD-41, 42, 43, 44, 45, 46, 47, 48, 49, 50, 51 |
| Pool 6 | RBD-52, 53, 54, 55, 56, 57, 58, 59, 60, 61 |

The results shown in FIG. 44 demonstrate that TECH001-CV021 and TECH001-CV023, encoding a first polypeptide comprising RBD (aa 319-542) from SARS-CoV-2 (Wuhan variant) in the antigenic unit and GM-CSF and IL-21, respectively, as a second protein, induced much stronger total T cell responses (FIG. 44A) against RBD than the comparison VB2060, encoding only the aforementioned first polypeptide. Moreover, TECH001-CV021 and TECH001-CV023 induced stronger CD8⁺ T cell responses (CD4+depleted splenocyte fraction) compared to VB2060 (FIG. 44B). TECH001-CV022 encoding the aforementioned first polypeptide and the two sub-domains of IL-12 as a second and third protein also induced much stronger total T cell responses (FIG. 44A) against RBD than VB2060. The increased T cell response induced by addition of the IL-12 cytokine appeared to be mainly due to an increase in IFN-γ secretion from CD4+ T cells as a greater reduction in response in the TECH001-CV022 CD4⁺ T cell depleted samples was observed compared to the TECH001-CV021 and TECH001-CV023 treated groups (FIG. 44B).

Taken together, the results presented demonstrate that the humoral and cellular immune responses elicited in mice against the SARS-CoV-2 RBD was enhanced by co-expressing a first polypeptide/dimeric protein comprising a targeting unit, a dimerization unit, and antigenic unit comprising an infectious antigen (RBD derived from the pathogen SARS-CoV-2) and an immunostimulatory compound (GM-CSF, IL-12, or IL-21) compared to expression of the aforementioned first polypeptide/dimeric protein alone.

### Sequence Overview

SEQ ID NO: 1
M¹QVSTAALAVLLCTMALCNQVLS²³A²⁴PLAADTPTACCFSYTSRQIPQNFIADYFETSS
QCSKPSVIFLTKRGRQVCADPSEEWVQKYVSDLELSA⁹³E⁹⁴LKTPLGDTTHT¹⁰⁵E¹⁰⁶PK
SCDTPPPCPRCP¹²⁰G¹²¹GGSSGGGSG¹³⁰G¹³¹QPREPQVYTLPPSREEMTKNQVSLTC
LVKGFYPSDIAVEWESSGQPENNYNTTPPMLDSDGSFFLYSKLTVDKSRWQQGNIFS
CSVMHEALHNRFTQKSLSLSPGK²³⁷
SEQ ID NO: 2
Signal peptide
MNFGLRLIFLVLTLKGVQC
SEQ ID NO: 3
Signal peptide
MDAMKRGLCCVLLLCGAVFVSP
SEQ ID NO: 4
Signal peptide of human FLT3L
MTVLAPAWSPTTYLLLLLLLSSGLSG
SEQ ID NO: 5
VB4194
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKSWIHCWKYLSVQSQLFRGSSLLFRRVGGGGSGGGGSNNLQK
YIEIYVQKINPSRLPVVIGGLLGGGGSGGGGSEVIQTSKYYMRDVIAIESAWLLELAPH
GGGGSGGGGSVILPQAPSGPSYATYLQPAQAQMLTPPGGGGSGGGGSFVSPMAHY
VPGIMAIESVVARFQFIVPGGGGSGGGGSGDVKIHAHKVVLANISPYFKAMFTGNLGG
GGSGGGGSTPLRKHTVHAIRKFYLEFKGSSPPPRLGGGGSGGGGSKIYEFDYHLYG
QNITMIMTSVSGHLLA
SEQ ID NO: 6
VB4168
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKSWIHCWKYLSVQSQLFRGSSLLFRRVGGGGSGGGGSNNLQK
YIEIYVQKINPSRLPVVIGGLLGGGGSGGGGSEVIQTSKYYMRDVIAIESAWLLELAPH
GGGGSGGGGSVILPQAPSGPSYATYLQPAQAQMLTPPGGGGSGGGGSFVSPMAHY
VPGIMAIESVVARFQFIVPGGGGSGGGGSGDVKIHAHKVVLANISPYFKAMFTGNLGG
GGSGGGGSTPLRKHTVHAIRKFYLEFKGSSPPPRLGGGGSGGGGSKIYEFDYHLYG
QNITMIMTSVSGHLLAGSGEGRGSLLTCGDVEENPGPMTVLAPAWSPTTYLLLLLLLS
SGLSGTQDCSFQHSPISSDFAVKIRELSDYLLQDYPVTVASNLQDEELCGGLWRLVLA
QRWMERLKTVAGSKMQGLLERVNTEIHFVTKCAFQPPPSCLRFVQTNISRLLQETSE
QLVALKPWITRQNFSRCLELQCQPDSSTLPPPWSPRPLEATAPTAPQP
SEQ ID NO: 7
VB4169
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKSWIHCWKYLSVQSQLFRGSSLLFRRVGGGGSGGGGSNNLQK
YIEIYVQKINPSRLPVVIGGLLGGGGSGGGGSEVIQTSKYYMRDVIAIESAWLLELAPH
GGGGSGGGGSVILPQAPSGPSYATYLQPAQAQMLTPPGGGGSGGGGSFVSPMAHY
VPGIMAIESVVARFQFIVPGGGGSGGGGSGDVKIHAHKVVLANISPYFKAMFTGNLGG
GGSGGGGSTPLRKHTVHAIRKFYLEFKGSSPPPRLGGGGSGGGGSKIYEFDYHLYG
QNITMIMTSVSGHLLAGSGEGRGSLLTCGDVEENPGPMTVLAPAWSPTTYLLLLLLLS
SGLSGTQDCSFQHSPISSDFAVKIRELSDYLLQDYPVTVASNLQDEELCGGLWRLVLA
QRWMERLKTVAGSKMQGLLERVNTEIHFVTKCAFQPPPSCLRFVQTNISRLLQETSE
QLVALKPWITRQNFSRCLELQCQPDSSTLPPPWSPRPLEATAPTAPQPGSGATNFSL
LKQAGDVEENPGPMWLQNLLFLGIVVYSLSAPTRSPITVTRPWKHVEAIKEALNLLDD
MPVTLNEEVEVVSNEFSFKKLTCVQTRLKIFEQGLRGNFTKLKGALNMTASYYQTYCP
PTPETDCETQVTTYADFIDSLKTFLTDIPFECKKPVQK
SEQ ID NO: 8
VB4170
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKSWIHCWKYLSVQSQLFRGSSLLFRRVGGGGSGGGGSNNLQK
YIEIYVQKINPSRLPVVIGGLLGGGGSGGGGSEVIQTSKYYMRDVIAIESAWLLELAPH
GGGGSGGGGSVILPQAPSGPSYATYLQPAQAQMLTPPGGGGSGGGGSFVSPMAHY
VPGIMAIESVVARFQFIVPGGGGSGGGGSGDVKIHAHKVVLANISPYFKAMFTGNLGG
GGSGGGGSTPLRKHTVHAIRKFYLEFKGSSPPPRLGGGGSGGGGSKIYEFDYHLYG
QNITMIMTSVSGHLLAGSGEGRGSLLTCGDVEENPGPMTVLAPAWSPTTYLLLLLLLS
SGLSGTQDCSFQHSPISSDFAVKIRELSDYLLQDYPVTVASNLQDEELCGGLWRLVLA
QRWMERLKTVAGSKMQGLLERVNTEIHFVTKCAFQPPPSCLRFVQTNISRLLQETSE
QLVALKPWITRQNFSRCLELQCQPDSSTLPPPWSPRPLEATAPTAPQPGSGATNFSL
LKQAGDVEENPGPMWLQNLLFLGIVVYSLSAPTRSPITVTRPWKHVEAIKEALNLLDD
MPVTLNEEVEVVSNEFSFKKLTCVQTRLKIFEQGLRGNFTKLKGALNMTASYYQTYCP
PTPETDCETQVTTYADFIDSLKTFLTDIPFECKKPVQKGSGQCTNYALLKLAGDVESNP
GPMKISAAALTIILTAAALCTPAPASPYGSDTTPCCFAYLSLALPRAHVKEYFYTSSKCS
NLAVVFVTRRNRQVCANPEKKWVQEYINYLEMS
SEQ ID NO: 9
T2A
EGRGSLLTCGDVEENPGP
SEQ ID NO: 10
Human FLT3L
TQDCSFQHSPISSDFAVKIRELSDYLLQDYPVTVASNLQDEELCGGLWRLVLAQRWM
ERLKTVAGSKMQGLLERVNTEIHFVTKCAFQPPPSCLRFVQTNISRLLQETSEQLVAL
KPWITRQNFSRCLELQCQPDSSTLPPPWSPRPLEATAPTAPQP
SEQ ID NO: 11
P2A
ATNFSLLKQAGDVEENPGP
SEQ ID NO: 12
Signal peptide mouse GM-CSF
MWLQNLLFLGIVVYSLS
SEQ ID NO: 13
Mouse GM-CSF
APTRSPITVTRPWKHVEAIKEALNLLDDMPVTLNEEVEVVSNEFSFKKLTCVQTRLKIF
EQGLRGNFTKLKGALNMTASYYQTYCPPTPETDCETQVTTYADFIDSLKTFLTDIPFEC
KKPVQK
SEQ ID NO: 14
E2A
QCTNYALLKLAGDVESNPGP
SEQ ID NO: 15
Signal peptide mouse CCL5
MKISAAALTIILTAAALCTPAPA
SEQ ID NO: 16
Mouse CCL5
SPYGSDTTPCCFAYLSLALPRAHVKEYFYTSSKCSNLAVVFVTRRNRQVCANPEKKW
VQEYINYLEMS
SEQ ID NO: 17
Linker
GGGGSGGGGS
SEQ ID NO: 18
VB4202
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKSWIHCWKYLSVQSQLFRGSSLLFRRVGGGGSGGGGSNNLQK
YIEIYVQKINPSRLPVVIGGLLGGGGSGGGGSEVIQTSKYYMRDVIAIESAWLLELAPH
GGGGSGGGGSVILPQAPSGPSYATYLQPAQAQMLTPPGGGGSGGGGSFVSPMAHY
VPGIMAIESVVARFQFIVPGGGGSGGGGSGDVKIHAHKVVLANISPYFKAMFTGNLGG
GGSGGGGSTPLRKHTVHAIRKFYLEFKGSSPPPRLGGGGSGGGGSKIYEFDYHLYG
QNITMIMTSVSGHLLAGSGEGRGSLLTCGDVEENPGPMWLQNLLFLGIVVYSLSAPTR
SPITVTRPWKHVEAIKEALNLLDDMPVTLNEEVEVVSNEFSFKKLTCVQTRLKIFEQGL
RGNFTKLKGALNMTASYYQTYCPPTPETDCETQVTTYADFIDSLKTFLTDIPFECKKPV
QK
SEQ ID NO: 19
VB1020
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQL
SEQ ID NO: 20
VB4195
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQLGSGEGRGSLLTCGD
VEENPGPMTVLAPAWSPTTYLLLLLLLSSGLSGTQDCSFQHSPISSDFAVKIRELSDYL
LQDYPVTVASNLQDEELCGGLWRLVLAQRWMERLKTVAGSKMQGLLERVNTEIHFV
TKCAFQPPPSCLRFVQTNISRLLQETSEQLVALKPWITRQNFSRCLELQCQPDSSTLP
PPWSPRPLEATAPTAPQP
SEQ ID NO: 21
VB4196
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQLGSGEGRGSLLTCGD
VEENPGPMTVLAPAWSPTTYLLLLLLLSSGLSGTQDCSFQHSPISSDFAVKIRELSDYL
LQDYPVTVASNLQDEELCGGLWRLVLAQRWMERLKTVAGSKMQGLLERVNTEIHFV
TKCAFQPPPSCLRFVQTNISRLLQETSEQLVALKPWITRQNFSRCLELQCQPDSSTLP
PPWSPRPLEATAPTAPQPGSGATNFSLLKQAGDVEENPGPMWLQNLLFLGIVVYSLS
APTRSPITVTRPWKHVEAIKEALNLLDDMPVTLNEEVEVVSNEFSFKKLTCVQTRLKIF
EQGLRGNFTKLKGALNMTASYYQTYCPPTPETDCETQVTTYADFIDSLKTFLTDIPFEC
KKPVQK
SEQ ID NO: 22
VB4204
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQLGSGEGRGSLLTCGD
VEENPGPMWLQNLLFLGIVVYSLSAPTRSPITVTRPWKHVEAIKEALNLLDDMPVTLNE
EVEVVSNEFSFKKLTCVQTRLKIFEQGLRGNFTKLKGALNMTASYYQTYCPPTPETDC
ETQVTTYADFIDSLKTFLTDIPFECKKPVQK
SEQ ID NO: 23
VB4205
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQLGSGEGRGSLLTCGD
VEENPGPMKISAAALTIILTAAALCTPAPASPYGSDTTPCCFAYLSLALPRAHVKEYFYT
SSKCSNLAVVFVTRRNRQVCANPEKKWVQEYINYLEMS
SEQ ID NO: 24
VB4208
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLGSGEGRGSLLTCGDVEENPGPMWLQNLLFLGIVVYSLSAPTRS
PITVTRPWKHVEAIKEALNLLDDMPVTLNEEVEVVSNEFSFKKLTCVQTRLKIFEQGLR
GNFTKLKGALNMTASYYQTYCPPTPETDCETQVTTYADFIDSLKTFLTDIPFECKKPVQ
K
SEQ ID NO 25:
Nucleotide sequence encoding amino acids 24-93 of SEQ ID NO: 1
GCACCACTTGCTGCTGACACGCCGACCGCCTGCTGCTTCAGCTACACCTCCCGA
CAGATTCCACAGAATTTCATAGCTGACTACTTTGAGACGAGCAGCCAGTGCTCCA
AGCCCAGTGTCATCTTCCTAACCAAGAGAGGCCGGCAGGTCTGTGCTGACCCCA
GTGAGGAGTGGGTCCAGAAATACGTCAGTGACCTGGAGCTGAGTGCC
SEQ ID NO: 26
Nucleotide sequence encoding amino acids 94-120 of SEQ ID NO: 1
GAGCTCAAAACCCCACTTGGTGACACAACTCACACAGAGCCCAAATCTTGTGACA
CACCTCCCCCGTGCCCAAGGTGCCCA
SEQ ID NO: 27:
Nucleotide sequence encoding amino acids 131-237 of SEQ ID NO: 1
GGACAGCCCCGAGAACCACAGGTGTACACCCTGCCCCCATCCCGGGAGGAGATG
ACCAAGAACCAGGTCAGCCTGACCTGCCTGGTCAAAGGCTTCTACCCCAGCGAC
ATCGCCGTGGAGTGGGAGAGCAGCGGGCAGCCGGAGAACAACTACAACACCAC
GCCTCCCATGCTGGACTCCGACGGCTCCTTCTTCCTCTACAGCAAGCTCACCGTG
GACAAGAGCAGGTGGCAGCAGGGGAACATCTTCTCATGCTCCGTGATGCATGAG
GCTCTGCACAACCGCTTCACGCAGAAGAGCCTCTCCCTGTCTCCGGGTAAA
SEQ ID NO: 28:
Nucleotide sequence encoding amino acids 94-237 of SEQ ID NO: 1
GAGCTCAAAACCCCACTTGGTGACACAACTCACACAGAGCCCAAATCTTGTGACA
CACCTCCCCCGTGCCCAAGGTGCCCAGGCGGTGGAAGCAGCGGAGGTGGAAGT
GGAGGACAGCCCCGAGAACCACAGGTGTACACCCTGCCCCCATCCCGGGAGGA
GATGACCAAGAACCAGGTCAGCCTGACCTGCCTGGTCAAAGGCTTCTACCCCAG
CGACATCGCCGTGGAGTGGGAGAGCAGCGGGCAGCCGGAGAACAACTACAACA
CCACGCCTCCCATGCTGGACTCCGACGGCTCCTTCTTCCTCTACAGCAAGCTCAC
CGTGGACAAGAGCAGGTGGCAGCAGGGGAACATCTTCTCATGCTCCGTGATGCA
TGAGGCTCTGCACAACCGCTTCACGCAGAAGAGCCTCTCCCTGTCTCCGGGTAAA
SEQ ID NO: 29
Nucleotide sequence encoding amino acids 1-23 of SEQ ID NO: 1
ATGCAGGTCTCCACTGCTGCCCTTGCCGTCCTCCTCTGCACCATGGCTCTCTGCA
ACCAGGTCCTCTCT
SEQ ID NO: 30
SARS-CoV-2 RBD (amino acids 319-542)
RVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNF
SEQ ID NO: 31
TECH001-CV021
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNFGSGEGRGSLLTCGDVEENPGPMWLQNLLFLGIVVYSLSAPTRSPITVTRPW
KHVEAIKEALNLLDDMPVTLNEEVEVVSNEFSFKKLTCVQTRLKIFEQGLRGNFTKLKG
ALNMTASYYQTYCPPTPETDCETQVTTYADFIDSLKTFLTDIPFECKKPVQK
SEQ ID NO: 32
TECH001-CV022
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNFGSGEGRGSLLTCGDVEENPGPMCQSRYLLFLATLALLNHLSLARVIPVSGP
ARCLSQSRNLLKTTDDMVKTAREKLKHYSCTAEDIDHEDITRDQTSTLKTCLPLELHKN
ESCLATRETSSTTRGSCLPPQKTSLMMTLCLGSIYEDLKMYQTEFQAINAALQNHNHQ
QIILDKGMLVAIDELMQSLNHNGETLRQKPPVGEADPYRVKMKLCILLHAFSTRVVTIN
RVMGYLSSAEGRGSLLTCGDVEENPGPMCPQKLTISWFAIVLLVSPLMAMWELEKDV
YVVEVDWTPDAPGETVNLTCDTPEEDDITWTSDQRHGVIGSGKTLTITVKEFLDAGQY
TCHKGGETLSHSHLLLHKKENGIWSTEILKNFKNKTFLKCEAPNYSGRFTCSWLVQRN
MDLKFNIKSSSSSPDSRAVTCGMASLSAEKVTLDQRDYEKYSVSCQEDVTCPTAEET
LPIELALEARQQNKYENYSTSFFIRDIIKPDPPKNLQMKPLKNSQVEVSWEYPDSWST
PHSYFSLKFFVRIQRKKEKMKETEEGCNQKGAFLVEKTSTEVQCKGGNVCVQAQDR
YYNSSCSKWACVPCRVRS
SEQ ID NO: 33
TECH001-CV023
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNFGSGEGRGSLLTCGDVEENPGPMERTLVCLVVIFLGTVAHKSSPQGPDRLLI
RLRHLIDIVEQLKIYENDLDPELLSAPQDVKGHCEHAAFACFQKAKLKPSNPGNNKTFII
DLVAQLRRRLPARRGGKKQKHIAKCPSCDSYEKRTPKEFLERLKWLLQKMIHQHLS
SEQ ID NO: 34
Mouse IL-12 A signal peptide
MCQSRYLLFLATLALLNHLSLA
SEQ ID NO: 35
Mouse IL-12 A
RVIPVSGPARCLSQSRNLLKTTDDMVKTAREKLKHYSCTAEDIDHEDITRDQTSTLKTC
LPLELHKNESCLATRETSSTTRGSCLPPQKTSLMMTLCLGSIYEDLKMYQTEFQAINA
ALQNHNHQQIILDKGMLVAIDELMQSLNHNGETLRQKPPVGEADPYRVKMKLCILLHA
FSTRVVTINRVMGYLSSA
SEQ ID NO: 36
Mouse IL-12 B signal peptide
MCPQKLTISWFAIVLLVSPLMA
SEQ ID NO: 37
Mouse IL-12 B
MWELEKDVYVVEVDWTPDAPGETVNLTCDTPEEDDITWTSDQRHGVIGSGKTLTITV
KEFLDAGQYTCHKGGETLSHSHLLLHKKENGIWSTEILKNFKNKTFLKCEAPNYSGRF
TCSWLVQRNMDLKFNIKSSSSSPDSRAVTCGMASLSAEKVTLDQRDYEKYSVSCQE
DVTCPTAEETLPIELALEARQQNKYENYSTSFFIRDIIKPDPPKNLQMKPLKNSQVEVS
WEYPDSWSTPHSYFSLKFFVRIQRKKEKMKETEEGCNQKGAFLVEKTSTEVQCKGG
NVCVQAQDRYYNSSCSKWACVPCRVRS
SEQ ID NO: 38
Mouse IL-21 signal peptide
MERTLVCLVVIFLGTVA
SEQ ID NO: 39
Mouse IL-21
HKSSPQGPDRLLIRLRHLIDIVEQLKIYENDLDPELLSAPQDVKGHCEHAAFACFQKAK
LKPSNPGNNKTFIIDLVAQLRRRLPARRGGKKQKHIAKCPSCDSYEKRTPKEFLERLK
WLLQKMIHQHLS
SEQ ID NO: 40
Human GM-CSF signal peptide
MWLQSLLLLGTVACSIS
SEQ ID NO: 41
Human GM-CSF
APARSPSPSTQPWEHVNAIQEARRLLNLSRDTAAEMNETVEVISEMFDLQEPTCLQT
RLELYKQGLRGSLTKLKGPLTMMASHYKQHCPPTPETSCATQIITFESFKENLKDFLLV
IPFDCWEPVQE
SEQ ID NO: 42
Human CCL5 signal peptide
MKVSAAALAVILIATALCAPASA
SEQ ID NO: 43
Human CCL5
SPYSSDTTPCCFAYIARPLPRAHIKEYFYTSGKCSNPAVVFVTRKNRQVCANPEKKWV
REYINSLEMS
SEQ ID NO: 44
Human IL-12A signal peptide
MCPARSLLLVATLVLLDHLSLA
SEQ ID NO: 45
Human IL-12A
RNLPVATPDPGMFPCLHHSQNLLRAVSNMLQKARQTLEFYPCTSEEIDHEDITKDKTS
TVEACLPLELTKNESCLNSRETSFITNGSCLASRKTSFMMALCLSSIYEDLKMYQVEFK
TMNAKLLMDPKRQIFLDQNMLAVIDELMQALNFNSETVPQKSSLEEPDFYKTKIKLCIL
LHAFRIRAVTIDRVMSYLNAS
SEQ ID NO: 46
Human IL-12B signal peptide
MCHQQLVISWFSLVFLASPLVA
SEQ ID NO: 47
Human IL-12B
IWELKKDVYVVELDWYPDAPGEMVVLTCDTPEEDGITWTLDQSSEVLGSGKTLTIQVK
EFGDAGQYTCHKGGEVLSHSLLLLHKKEDGIWSTDILKDQKEPKNKTFLRCEAKNYS
GRFTCWWLTTISTDLTFSVKSSRGSSDPQGVTCGAATLSAERVRGDNKEYEYSVEC
QEDSACPAAEESLPIEVMVDAVHKLKYENYTSSFFIRDIIKPDPPKNLQLKPLKNSRQV
EVSWEYPDTWSTPHSYFSLTFCVQVQGKSKREKKDRVFTDKTSATVICRKNASISVR
AQDRYYSSSWSEWASVPCS
SEQ ID NO: 48
Human IL-21 signal peptide
MRSSPGNMERIVICLMVIFLGTLV
SEQ ID NO: 49
Human IL-21
HKSSSQGQDRHMIRMRQLIDIVDQLKNYVNDLVPEFLPAPEDVETNCEWSAFSCFQK
AQLKSANTGNNERIINVSIKKLKRKPPSTNAGRRQKHRLTCPSCDSYEKKPPKEFLER
FKSLLQKMIHQHLSSRTHGSEDS

### Embodiments

1. A vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, such as one or more disease-relevant antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

2. The vector according to embodiment 1, wherein the one or more immunostimulatory compounds are compounds that affect antigen-presenting cells, including dendritic cells, macrophages, Langerhans cells, B cells and neutrophils, such as compounds that stimulate antigen-presenting cells, preferably wherein the one or more immunostimulatory compounds are compounds that affect human antigen-presenting cells, including human dendritic cells, human macrophages, human Langerhans cells, human B cells and human neutrophils

3. The vector according to embodiment 1 or 2, wherein the one or more immunostimulatory compounds promote attraction and/or activation and/or maturation and/or proliferation, such as growth and/or expansion, of antigen-presenting cells.

4. The vector according to any of embodiments 1 to 3, wherein the one or more immunostimulatory compounds promote attraction of antigen-presenting cells.

5. The vector according to embodiment 4, wherein the one or more immunostimulatory compounds are chemokines, preferably human chemokines.

6. The vector according to embodiment 5, wherein the one or more immunostimulatory compounds can interact with a surface molecule on an antigen-presenting cell selected from the group consisting of CCR1, CCR3, CCR4, CCR5, CCR6, CCR7, CCR8 and XCR1, preferably wherein the one or more immunostimulatory compounds can interact with a surface molecule on a human antigen-presenting cell selected from the group consisting of hCCR1, hCCR3, hCCR4, hCCR5, hCCR6, hCCR7, hCCR8 and hXCR1.

7. The vector according to any of embodiments 5 to 6, wherein the one or more immunostimulatory compounds are selected from the list consisting of macrophage inflammatory protein alpha, including its isoforms, such as mouse CCL3, human CCL3, human CCL3L1, human CCL3L2 and human CCL3L3, CCL4, preferably hCCL4, CCL5, preferably hCCL5, CCL19, preferably hCCL19, CCL20, preferably hCCL20, CCL21, preferably hCCL21, XCL1, preferably hXCL1 and XCL2, preferably hXCL2.

8. The vector according to any of embodiments 3 to 7, wherein the one or more immunostimulatory compounds promote activation and/or maturation of antigen-presenting cells.

9. The vector according to any of embodiments 3 to 8, wherein the one or more immunostimulatory compounds can interact with a surface molecule on an antigen-presenting cell which is selected from the group consisting of a receptor of the TNF receptor superfamily, including CD40 (cluster of differentiation 40), CD137 (4-1BB), CD27, RANK, and ICOS (CD278), preferably wherein the one or more immunostimulatory compounds can interact with a surface molecule on a human antigen-presenting cell which is selected from the group consisting of a receptor of the human TNF receptor superfamily, including human CD40, hCD137, hCD27, hRANK, and hICOS.

10. The vector according to embodiment 9, wherein the one or more immunostimulatory compounds are selected from the list consisting of CD40L, CD137L, CD70, RANKL and ICOSL, preferably wherein the one or more immunostimulatory compounds are selected from the list consisting of hCD40L, hCD137L, hCD70, hRANKL and hICOSL.

11. The vector according to any of embodiments 3 to 8, wherein the one or more immunostimulatory compounds are cytokines selected from the group consisting of IL-2, IL-10, IL-12, IL-21, TNFα, IFNγ and IL-1β, preferably wherein the one or more immunostimulatory compounds are human cytokines selected from the group consisting of hIL-2, hIL-10, hIL-12, hIL-21, hTNFα, hIFNγ and hIL-1B.

12. The vector according to any of embodiments 3 to 8, wherein the one or more immunostimulatory compounds are viral infection sensors, such as MyD88 or TRIF, preferably human viral infection sensors, such as human MyD88 or human TRIF.

13. The vector according to any of embodiments 3 to 8, wherein the one or more immunostimulatory compounds can interact with a pattern-recognition receptor on an antigen-presenting cell, such as a Toll-like receptor, including TLR2, TLR4, TLR5 and TLR9 and/or with a receptor on an antigen-presenting cell selected from the group consisting of RAGE, TIM-3, FPR, SREC1, LOX1 and CD91, preferably wherein the one or more immunostimulatory compounds can interact with a pattern-recognition receptor on a human antigen-presenting cell, such as a human Toll-like receptor, including hTLR2, hTLR4, hTLR5 and hTLR9 and/or with a receptor on a human antigen-presenting cell selected from the group consisting of hRAGE, hTIM-3, hFPR, hSREC1, hLOX1 and hCD91.

14. The vector according to embodiment 13, wherein the one or more immunostimulatory compounds are selected from the group consisting of pathogen-associated molecular patterns (PAMPs), such as flagellin, protein damage-associated molecular patterns (DAMPs), such as HMGB1, heat-shock proteins (HSPs), Calrecticulin and Annexin A1, preferably wherein the one or more immunostimulatory compounds are selected from the group consisting of human pathogen-associated molecular patterns (PAMPs), human protein damage-associated molecular patterns (DAMPs), such as HMGB1, human heat-shock proteins (HSPs), human Calrecticulin and human Annexin A1.

15. The vector according to any of embodiments 3 to 14, wherein the one or more immunostimulatory compounds promote growth and/or expansion of antigen-presenting cells.

16. The vector according to any of embodiments 3 to 15 wherein the one or more immunostimulatory compounds are growth factors, preferably human growth factors.

17. The vector according to any of embodiments 3 to 15, wherein the one or more immunostimulatory compounds can interact with a surface molecule on an antigen-presenting cell which is selected from the group consisting of GM-CSF-receptor, FLT-3R, IL-15R and IL-4R, preferably wherein the one or more immunostimulatory compounds can interact with a surface molecule on a human antigen-presenting cell which is selected from the group consisting of hGM-CSF-receptor, hFLT-3R, hIL-15R and hIL-4R.

18. The vector according to any of embodiments 16 to 17, wherein the one or more immunostimulatory compounds are selected from the group consisting of GM-CSF, FLT-3L, IL-15 and IL-4, preferably wherein the one or more immunostimulatory compounds are selected from the group consisting of hGM-CSF, hFLT-3L, hIL-15 and hIL-4.

19. The vector according to any of embodiments 1 to 17, wherein the one or more immunostimulatory compounds are selected from the list consisting of IL-4, IL-1β, IFNγ, IFNα, IL-15, TNFα, IL-10, IL-12, IL-21, IL-2, MyD88, TRIF, RIG-I, MDA-5, P28 region of C3d, IL-13, IFNε, IFNκ, IFNω, IFNβ and IL-6, preferably wherein the one or more immunostimulatory compounds are selected from the list consisting of hIL-4, hIL-1ß, hIFNγ, hIFNα,h IL-15, hTNFα, hIL-10, hIL-12, hIL-21, hIL-2, hMyD88, hTRIF, hRIG-I, hMDA-5, P28 region of hC3d, hIL-13, hIFNε, hIFNκ, hIFNω, hIFNβ and hIL-6.

20. The vector according to any of embodiments 1 to 19 comprising multiple further nucleic acid sequences encoding multiple immunostimulatory compounds, such as 2, 3, 4, 5, 6, 7 or 8 immunostimulatory compounds, such as 2, 3, 4, 5, 6, 7 or 8 different immunostimulatory compounds.

21. The vector according to embodiment 20, wherein said multiple immunostimulatory compounds are different immunostimulatory compounds which affect, such as stimulate, antigen-presenting cells differently.

22. The vector according to any of the previous embodiments, wherein said vector comprises one or more co-expression elements.

23. The vector according to embodiment 22, wherein said one or more co-expression elements cause the transcription of the first polypeptide and the one or more immunostimulatory compounds on a single transcript and the independent translation into a separate first polypeptide and separate one or more immunostimulatory compounds.

24. The vector according to any of embodiments 22 to 23, wherein the one or more co-expression elements are IRES elements or nucleic acid sequences encoding 2A self-cleaving peptides.

25. The vector according to any of embodiments 22 to 23, wherein said vector comprises more than one co-expression element which are IRES elements or nucleic acid sequences encoding 2A self-cleaving peptides or IRES elements and nucleic acid sequences encoding 2A self-cleaving peptides.

26. The vector according to any of embodiments 22 to 25, wherein the 2A self-cleaving peptide is selected from the group consisting of T2A peptide, P2A peptide, E2A peptide and F2A peptide.

27. The vector according to any of embodiments 22 to 26, wherein the 2A self-cleaving peptide is selected from the group consisting of T2A peptide having an amino acid sequence which has 80% to 100% sequence identity to the amino acid sequence with SEQ ID NO: 9, P2A peptide having an amino acid sequence which has 80% to 100% sequence identity to the amino acid sequence with SEQ ID NO: 11, E2A peptide having an amino acid sequence which has 80% to 100% sequence identity to the amino acid sequence with SEQ ID NO: 14 and F2A peptide having an amino acid sequence which has 80% to 100% sequence identity to the amino acid sequence with SEQ ID NO: 51.

28. The vector according to any of embodiments 22 to 27, wherein the 2A self-cleaving peptide is selected from the group consisting of T2A peptide having an amino acid sequence with SEQ ID NO: 9, P2A peptide having an amino acid sequence with SEQ ID NO: 11, E2A peptide having an amino acid sequence with SEQ ID NO: 14 and F2A peptide having an amino acid sequence with SEQ ID NO: 51.

29. The vector according to embodiment 23, wherein said one or more co-expression elements cause the transcription of the first polypeptide and the one or more immunostimulatory compounds as separate transcripts.

30. The vector according to embodiment 29, wherein said one or more co-expression elements are bidirectional promoters.

31. The vector according to embodiment 29, wherein said one or more co-expression elements are promoters and wherein the vector comprises a separate promoter for each of the nucleic acid sequences encoding the first polypeptide and the one or more immunostimulatory compounds.

32. The vector according to embodiment 29, wherein said one or more co-expression elements are bidirectional promoters and promoters.

33. The vector according to any of embodiments 23 to 33, wherein said vector comprises one or more co-expression elements selected from the group consisting of IRES elements, nucleic acid sequences encoding 2A self-cleaving peptides, bidirectional promoters and promoters.

34. The vector according to any of embodiments 1 to 33, wherein the antigenic unit comprises one or more neoantigens or parts thereof.

35. The vector according to embodiment 34, wherein the antigenic unit comprises one or more parts of one or more neoantigens.

36. The vector according to embodiment 35, wherein said parts are neoepitopes.

37. The vector according to embodiment 36, wherein the antigenic unit comprises several neoepitopes, such as several neoepitopes which are separated from each other by linkers.

38. The vector according to any of embodiments 36 to 37, wherein the antigenic unit comprises n−1 antigenic subunits, each subunit comprising a neoepitope and a subunit linker, and a terminal neoepitope, and wherein n is the number of neoepitopes in said antigenic unit and n is an integer of from 1 to 50.

39. The vector according to any of embodiments 36 to 38, wherein the neoepitopes have a length of from 7 to 30 amino acids such as from 7 to 10 amino acids (such as 7, 8, 9 or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

40. The vector according to any of embodiments 34 to 35, wherein the antigenic unit further comprises one or more patient-present shared cancer antigens or parts thereof.

41. The vector according to embodiment 40, wherein the antigenic unit further comprises one or more parts of one or more patient-present shared cancer antigens.

42. The vector according to embodiment 41, wherein said parts are epitopes.

43. The vector according to any of embodiments 40 to 41, wherein the antigenic unit further comprises several patient-present shared cancer epitopes.

44. The vector according to any of embodiments 40 to 43, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

45. The vector according to any of embodiments 1 to 33, wherein the antigenic unit comprises one or more patient-present shared cancer antigens or parts thereof.

46. The vector according to embodiment 45, wherein the antigenic unit comprises one or more parts of one or more patient-present shared cancer antigens.

47. The vector according to embodiment 46, wherein said parts are epitopes.

48. The vector according to embodiment 47, wherein the antigenic unit comprises several epitopes.

49. The vector according to any of embodiments 45 to 46, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

50. The vector according to any of embodiments 1 to 33, wherein the antigenic unit comprises one or more shared cancer antigens or parts thereof.

51. The vector according to embodiment 50, wherein the antigenic unit comprises one or more parts of one or more shared cancer antigens.

52. The vector according to embodiment 51, wherein said parts are epitopes.

53. The vector according to embodiment 52, wherein the antigenic unit comprises several epitopes.

54. The vector according to any of embodiments 50 to 53, wherein the shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations, scFvs derived from a monoclonal Ig produced by myeloma or lymphoma, telomerase, HIV antigens, tyrosinase, tyrosinase related protein (TRP)-1, TRP-2, melanoma antigen, prostate specific antigen and HPV antigens.

55. The vector according to any of embodiments 1 to 33, wherein the antigenic unit comprises one or more infectious antigens or parts thereof.

56. The vector according to embodiment 55, wherein the antigenic unit comprises one or more full-length infectious antigens or one or more parts of such full-lengths infectious antigens or one or more full-length infectious antigens and one or more parts of such full-lengths infectious antigens.

57. The vector according to any of embodiments 55 to 56, wherein the antigenic unit comprises one or more parts of one or more infectious antigens.

58. The vector according to embodiment 57, wherein such parts are B cell epitopes, such that the antigenic unit comprises one or more B cell epitopes from one or more infectious antigens.

59. The vector according to embodiment 57, wherein such parts are T cell epitopes, such that the antigenic unit comprises one or more T cell epitopes from one or more infectious antigens.

60. The vector according to any of embodiment 55 to 56, wherein the antigenic unit comprises (i) one or more full-length infectious antigens or one or more parts of such antigens and (ii) one or more T cell epitopes from one or more infectious antigens.

61. The vector according to embodiment 60, wherein the antigenic unit comprises a subunit comprising the one or more T cell epitopes which are separated from each other by subunit linkers, if more than one T cell epitope is comprised in the subunit; and wherein the subunit is connected to the multimerization unit by a first linker, such as a unit linker and separated from the one or more full-length infectious antigens or parts of such antigens by a second linker.

62. The vector according to any of embodiments 1 to 33, wherein the antigenic unit comprises one or more antigens derived from one or more pathogens or parts of such antigens.

63. The vector according to embodiment 62, wherein the antigenic unit comprises one or more full-length antigens derived from one or more pathogens or one or more parts of such full-lengths antigens or one or more full-length antigens derived from one or more pathogens and one or more parts of such full-lengths antigens.

64. The vector according to any of embodiments 62 to 63, wherein the antigenic unit comprises one or more parts of one or more antigens derived from one or more pathogens.

65. The vector according to embodiment 64, wherein such parts are B cell epitopes, such that the antigenic unit comprises one or more B cell epitopes derived from one or more pathogens.

66. The vector according to embodiment 64, wherein such parts are T cell epitopes, such that the antigenic unit comprises one or more T cell epitopes derived from one or more pathogens.

67. The vector according to any of embodiment 62 to 63, wherein the antigenic unit comprises (i) one or more full-length antigens derived from one or more pathogens or one or more parts of such antigens and (ii) one or more T cell epitopes derived from one or more pathogens.

68. The vector according to embodiment 67, wherein the antigenic unit comprises a subunit comprising the one or more T cell epitopes which are separated from each other by subunit linkers, if more than one T cell epitope is comprised in the subunit; and wherein the subunit is connected to the multimerization unit by a first linker, such as a unit linker and separated from the one or more full-length infectious antigens or parts of such antigens by a second linker.

69. The vector according to any of embodiments 62 to 68, wherein the one or more pathogens are selected from the group consisting of viruses, bacteria, fungi and parasites.

70. The vector according to any of embodiments 55 to 68, wherein the one or more antigens are selected from the group consisting of tuberculosis antigens, brucellosis antigens such as OMP31, HIV antigens such as gp120 derived sequences, HSV-2 antigens such as glycoprotein D, influenza virus antigens such as hemagglutinin, nucleoprotein and M2, HPV antigens such as E1, E2, E6, E7, L1 or, L2, e.g. E6 and E7 of HPV16 or HPV18, CMV antigens, HBV antigens, betacoronavirus antigens, such as SARS-CoV antigens, MERS-CoV antigens and SARS-CoV-2 antigens.

71. The vector according to any of the previous embodiments, wherein the antigenic unit comprises up to 3500 amino acids, such as from about 21 to about 2000 amino acids or from about 60 to 3500 amino acids, such as from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

72. The vector according to any of the previous embodiments, wherein the targeting unit is or comprises a moiety that interacts with surface molecules on the antigen-presenting cells, preferably wherein the targeting unit is or comprises a moiety that interacts with surface molecules on human antigen-presenting cells.

73. The vector according to embodiment 72, wherein the surface molecule is selected from the group consisting of MHC, CD14, CD40, CLEC9A, chemokine receptors, such as CCR1, CCR3, CCR4, CCR5, CCR6, CCR7, CCR8 and XCR1 and Toll-like receptors such as TLR-2, TLR-4 or TLR-5, preferably wherein the surface molecule is selected from the group consisting of HLA; hCD14, hCD40, hCLEC9A, human chemokine receptors, such as hCCR1, hCCR3, hCCR4, hCCR5, hCCR6, hCCR7, hCCR8 and hXCR1 and Toll-like receptors such as hTLR-2, hTLR-4 or hTLR-5.

74. The vector according to any of embodiments 72 and 73, wherein the targeting unit comprises or consists of soluble CD40 ligand, preferably human soluble CD40 ligand, CCL4 and its isoforms, preferably human CCL4 and its isoforms, CCL5, preferably human CCL5, CCL19, preferably human CCL19, CCL20, preferably human CCL20, CCL21, preferably human CCL21, macrophage inflammatory protein alpha including its isoforms, such as mouse CCL3, human CCL3, human CCL3L1, human CCL3L2 and human CCL3L3, XCL1, preferably human XCL1, XCL2, preferably human XCL2, flagellin, anti-HLA-DP, anti-HLA-DR, anti-pan HLA class II, anti-CD40, preferably anti-human CD40, anti-TLR-2, preferably anti-human TLR-2, anti-TLR-4, preferably anti-human TLR-4, anti-TLR-5, preferably anti-human TLR-5 or anti-CLEC9A, preferably anti-human CLEC9A.

75. The vector according to embodiment 74, wherein the targeting unit comprises or consists of human MIP-1α (LD78B, CCL3L1).

76. The vector according to embodiment 75, wherein the targeting unit comprises an amino acid sequence having at least 80% sequence identity to the amino acid sequence 24-93 of SEQ ID NO: 1, such as comprising the amino acid sequence 26-93 of SEQ ID NO: 1 or comprising the amino acid sequence 28-93 of SEQ ID NO: 1.

77. The vector according to embodiment 76, wherein the targeting unit consists of an amino acid sequence having at least 80% sequence identity to the amino acid sequence 24-93 of SEQ ID NO: 1, such as consisting of the amino acid sequence 26-93 of SEQ ID NO: 1 or consisting of the amino acid sequence 28-93 of SEQ ID NO: 1.

78. The vector according to embodiment 77, wherein the targeting unit consists of the amino acid sequence 24-93 of SEQ ID NO: 1.

79. The vector according to any of the previous embodiments, wherein the multimerization unit is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4.

80. The vector according to embodiment 79, wherein the vector comprises a hinge region which has the ability to form one or more covalent bonds.

81. The vector according to any of embodiments 79 to 80, wherein the hinge region is Ig derived.

82. The vector according to any of embodiments 79 to 81, wherein the multimerization unit is a dimerization unit and said dimerization unit further comprises another domain that facilitates dimerization.

83. The vector according to embodiment 82, wherein the other domain is an immunoglobulin domain, preferably an immunoglobulin constant domain.

84. The vector according to any of embodiments 82 to 83, wherein the other domain is a carboxyterminal C domain derived from IgG, preferably from IgG3.

85. The vector according to any of embodiments 82 to 84, wherein the dimerization unit further comprises a dimerization unit linker, such as glycine-serine rich linker, such as GGGSSGGGSG (SEQ ID NO: 134).

86. The vector according to embodiment 85, wherein the dimerization unit linker connects the hinge region and the other domain that facilitates dimerization.

87. The vector according to any of embodiments 82 to 86, wherein the dimerization unit comprises hinge exon h1 and hinge exon h4, a dimerization unit linker and a CH3 domain of human IgG3.

88. The vector according to embodiment 87, wherein the dimerization unit comprises an amino acid sequence having at least 80% sequence identity to the amino acid sequence 94-237 of SEQ ID NO: 1.

89. The vector according to embodiment 88, wherein the dimerization unit consists of an amino acid sequence having at least 80% sequence identity to the amino acid sequence 94-237 of SEQ ID NO: 1.

90. The vector according to embodiment 89, wherein the dimerization unit consists of the amino acid sequence 94-237 of SEQ ID NO: 1.

91. The vector according to any of the previous embodiments, wherein the first nucleic acid sequence encodes a first polypeptide which further comprises a unit liker that connects the antigenic unit to the multimerization unit, and wherein the unit linker is a non-immunogenic linker and/or flexible or rigid linker. 92. The vector according to any of the previous embodiments, wherein the first nucleic acid sequence encodes a first polypeptide which further comprises a signal peptide, preferably wherein the signal peptide is the natural leader sequence of the protein which is the targeting unit.

93. The vector according to embodiment 92, wherein the signal peptide is selected from the group consisting of Ig VH signal peptide, human TPA signal peptide and human MIP-1α signal peptide.

94. The vector according to any of embodiments 92 to 93, wherein the targeting unit is human MIP-1α and the signal peptide comprises an amino acid sequence having at least 85% sequence identity to the amino acid sequence 1-23 of SEQ ID NO: 1.

95. The vector according to embodiment 94, wherein the signal peptide consists of an amino acid sequence having at least 85% sequence identity to the amino acid sequence 1-23 of SEQ ID NO: 1.

96. The vector according to embodiment 95, wherein the signal peptide consists of the amino acid sequence 1-23 of SEQ ID NO: 1.

97. The vector according to any of the previous embodiments, wherein the one or more further nucleic acid sequences further encode a signal peptide.

98. The vector according to embodiment 97, wherein the signal peptide is the natural leader sequence of the immunostimulatory compound(s).

99. The vector according to any of the previous embodiments, wherein the vector is a viral vector, such as an RNA viral vector or DNA viral vector or a plasmid, such as an RNA plasmid or DNA plasmid.

100. A method of producing a vector as defined in any of embodiments 1 to 99, the method comprising:
a) transfecting cells in vitro with the vector;
b) culturing said cells;
c) optionally, lysing the cells to release the vector from the cells; and
d) collecting and optionally purifying the vector.

101. A host cell comprising a vector as defined in any of embodiments 1 to 99, such as a host cell selected from the group consisting of prokaryote cells, yeast cells, insect cells, higher eukaryotic cells such as cells from animals or humans.

102. A vector as defined in any of embodiments 1 to 99 for use as a medicament 103. A pharmaceutical composition comprising the vector as defined in any of embodiments 1 to 99 and a pharmaceutically acceptable carrier or diluent.

104. The pharmaceutical composition according to embodiment 103, wherein the pharmaceutically acceptable carrier or diluent is selected from the group consisting of saline, buffered saline, such as PBS, dextrose, water, glycerol, ethanol, isotonic aqueous buffers and Tyrode's buffer and combinations thereof.

105. The pharmaceutical composition according to any of embodiments 103 to 104, wherein the composition further comprises a transfection agent.

106. The composition according to any of embodiments 103 to 105 wherein the composition further comprises a pharmaceutically acceptable amphiphilic block co-polymer comprising blocks of poly(ethylene oxide) and polypropylene oxide), such as further comprises a pharmaceutically acceptable amphiphilic block co-polymer comprising blocks of poly(ethylene oxide) and polypropylene oxide) in an amount of from 0.2% w/v to 20% w/v.

107. The pharmaceutical composition according to any of embodiments 103 to 106, wherein the composition comprises said vector, e.g. said DNA plasmid, in a range of from 0.1 to 10 mg.

108. A method of treating a subject having a disease or being in need of prevention of said disease, the method comprising administering to the subject a vector as defined in any of embodiments 1 to 99 or a pharmaceutical composition as defined in any of embodiments 103 to 108.

109. The method according to embodiment 108, wherein the vector or the pharmaceutical composition is administered in a therapeutically or prophylactically effective amount.

110. The method according to any of embodiments 108 to 109, wherein the vector or the pharmaceutical composition is administered by intradermal, intramuscular, or subcutaneous injection, or by mucosal or epithelial application, such as intranasal or oral.

111. A method of treating a subject having cancer, the method comprising administering to the subject a vector as defined in any of embodiments 1 to 54 and 71 to 99 or a pharmaceutical composition as defined in any of embodiments 103 to 107 comprising such vector.

112. The method according to embodiment 111, wherein the vector or the pharmaceutical composition is administered in a therapeutically effective amount.

113. The method according to any of embodiments 111 to 112, wherein the vector or the pharmaceutical composition is administered by intradermal, intramuscular, or subcutaneous injection, or by mucosal or epithelial application, such as intranasal or oral.

114. The method according to any of embodiments 111 to 113, wherein the cancer is a liquid or solid cancer, such as a cancer selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

115. A method for treating a subject having an infectious disease or being in need of prevention of an infectious disease, the method comprising administering to the subject a vector as defined in any of embodiments 1 to 33, 55 to 70 and 71 to 99 or a pharmaceutical composition as defined in any of embodiments 103 to 107, comprising such vector.

116. The method according to embodiment 115, wherein the vector or the pharmaceutical composition is administered in a therapeutically or prophylactically effective amount.

117. The method according to any of embodiments 116 to 116, wherein the vector or the pharmaceutical composition is administered by intradermal, intramuscular, or subcutaneous injection, or by mucosal or epithelial application, such as intranasal or oral.

1. A vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more antigens or parts thereof, such as one or more disease-relevant antigens or parts thereof; and
(b) one or more further nucleic acid sequences encoding one or more immunostimulatory compounds,
wherein the vector allows for the co-expression of the first polypeptide and the one or more immunostimulatory compounds as separate molecules.

2. The vector according to claim 1, wherein the one or more immunostimulatory compounds promote attraction and/or activation and/or maturation and/or proliferation, such as growth and/or expansion, of antigen-presenting cells, preferably of human antigen-presenting cells.

3. The vector according to any of claims 1 to 2, wherein the one or more immunostimulatory compounds promote attraction of antigen-presenting cells.

4. The vector according to claim 3, wherein the one or more immunostimulatory compounds are chemokines, preferably human chemokines.

5. The vector according to claim 4, wherein the one or more immunostimulatory compounds can interact with a surface molecule on an antigen-presenting cell selected from the group consisting of CCR1, CCR3, CCR4, CCR5, CCR6, CCR7, CCR8 and XCR1, preferably wherein the one or more immunostimulatory compounds can interact with a surface molecule on a human antigen-presenting cell selected from the group consisting of hCCR1, hCCR3, hCCR4, hCCR5, hCCR6, hCCR7, hCCR8 and hXCR1.

6. The vector according to any of claims 4 to 5, wherein the one or more immunostimulatory compounds are selected from the list consisting of macrophage inflammatory protein alpha, including its isoforms, such as mouse CCL3, human CCL3, human CCL3L1, human CCL3L2 and human CCL3L3, CCL4, preferably human CCL4, CCL5, preferably human CCL5, CCL19, preferably human CCL19, CCL20, preferably human CCL20, CCL21, preferably human CCL21, XCL1, preferably human XCL1 and XCL2, preferably humanXCL2.

7. The vector according to any of claims 2 to 6, wherein the one or more immunostimulatory compounds promote activation and/or maturation of antigen-presenting cells.

8. The vector according to any of claims 2 to 7, wherein the one or more immunostimulatory compounds can interact with a surface molecule on an antigen-presenting cell which is selected from the group consisting of a receptor of the TNF receptor superfamily, including CD40 (cluster of differentiation 40), CD137 (4-1BB), CD27, RANK, and ICOS (CD278), preferably wherein the one or more immunostimulatory compounds can interact with a surface molecule on a human antigen-presenting cell which is selected from the group consisting of a receptor of the human TNF receptor superfamily, including hCD40, hCD137, hCD27, hRANK and hICOS.

9. The vector according to claim 8, wherein the one or more immunostimulatory compounds are selected from the list consisting of CD40L, CD137L, CD70, RANKL and ICOSL, preferably wherein the one or more immunostimulatory compounds are selected from the list consisting of hCD40L, hCD137L, hCD70, hRANKL and hICOSL.

10. The vector according to any of claims 2 to 7, wherein the one or more immunostimulatory compounds are cytokines selected from the group consisting of IL-2, IL-10, IL-12, IL-21, TNFα, IFNγ and IL-1β, preferably wherein the one or more immunostimulatory compounds are human cytokines selected from the group consisting of hIL-2, IhL-10, hIL-12, hIL-21, hTNFα, hIFNγ and hIL-1β.

11. The vector according to any of claims 2 to 7, wherein the one or more immunostimulatory compounds are viral infection sensors, such as MyD88 or TRIF, preferably human viral infection sensors, such as human MyD88 or human TRIF.

12. The vector according to any of claims 2 to 7, wherein the one or more immunostimulatory compounds can interact with a pattern-recognition receptor on an antigen-presenting cell, such as a Toll-like receptor, including TLR2, TLR4, TLR5 and TLR9 and/or with a receptor on an antigen-presenting cell selected from the group consisting of RAGE, TIM-3, FPR, SREC1, LOX1 and CD91, preferably wherein the one or more immunostimulatory compounds can interact with a pattern-recognition receptor on a human antigen-presenting cell, such as a human Toll-like receptor, including hTLR2, hTLR4, hTLR5 and hTLR9 and/or with a receptor on a human antigen-presenting cell selected from the group consisting of hRAGE, hTIM-3, hFPR, hSREC1, hLOX1 and hCD91.

13. The vector according to claim 12, wherein the one or more immunostimulatory compounds are selected from the group consisting of pathogen-associated molecular patterns (PAMPs), such as flagellin, protein damage-associated molecular patterns (DAMPs), such as HMGB1, heat-shock proteins (HSPs), Calrecticulin and Annexin A1, preferably wherein the one or more immunostimulatory compounds are selected from the group consisting of human pathogen-associated molecular patterns (PAMPs), human protein damage-associated molecular patterns (DAMPs), such as hHMGB1, human heat-shock proteins (HSPs), human Calrecticulin and human Annexin A1.

14. The vector according to any of claims 2 to 13, wherein the one or more immunostimulatory compounds promote growth and/or expansion of antigen-presenting cells.

15. The vector according to any of claims 2 to 14 wherein the one or more immunostimulatory compounds are growth factors, preferably human growth factors.

16. The vector according to any of claims 2 to 14, wherein the one or more immunostimulatory compounds can interact with a surface molecule on an antigen-presenting cell which is selected from the group consisting of GM-CSF-receptor, FLT-3R, IL-15R and IL-4R, preferably wherein the one or more immunostimulatory compounds can interact with a surface molecule on a human antigen-presenting cell which is selected from the group consisting of hGM-CSF-receptor, hFLT-3R, hIL-15R and hIL-4R.

17. The vector according to any of claims 15 to 16, wherein the one or more immunostimulatory compounds are selected from the group consisting of GM-CSF, FLT-3L, IL-15 and IL-4, preferably wherein the one or more immunostimulatory compounds are selected from the group consisting of hGM-CSF, hFLT-3L, hIL-15 and hIL-4.

18. The vector according to any of the previous claims, wherein the one or more immunostimulatory compounds are selected from the list consisting of IL-4, IL-1β, IFNγ, IFNα, IL-15, TNFα, IL-10, IL-12, IL-2, IL-21, MyD88, TRIF, RIG-I, MDA-5, P28 region of C3d, IL-13, IFNε, IFNκ, IFNω, IFNβ and IL-6, preferably wherein the one or more immunostimulatory compounds are selected from the list consisting of hIL-4, hIL-1β, hIFNγ, hIFNα, hIL-15, hTNFα, hIL-10, hIL-12, hIL-2, hIL-21, hMyD88, hTRIF, hRIG-I, hMDA-5, P28 region of hC3d, hIL-13, hIFNε, hIFNκ, hIFNω, hIFNβ and hIL-6.

19. The vector according to any of the previous claims, wherein the vector comprises multiple further nucleic acid sequences encoding more than one immunostimulatory compound, such as 2, 3, 4, 5, 6, 7 or 8 immunostimulatory compounds, such as 2, 3, 4, 5, 6, 7 or 8 different immunostimulatory compounds.

20. The vector according to claim 19, wherein said multiple immunostimulatory compounds are different immunostimulatory compounds which affect, such as stimulate, antigen-presenting cells differently.

21. The vector according to any of the previous claims, wherein said vector comprises one or more co-expression elements.

22. The vector according to claim 21, wherein said one or more co-expression elements cause the transcription of the first polypeptide and the one or more immunostimulatory compounds on a single transcript and the independent translation into a separate first polypeptide and separate one or more immunostimulatory compounds.

23. The vector according to any of claims 21 to 22, wherein the one or more co-expression elements are IRES elements or nucleic acid sequences encoding 2A self-cleaving peptides.

24. The vector according to claim 21, wherein said one or more co-expression elements cause the transcription of the first polypeptide and the one or more immunostimulatory compounds as separate transcripts.

25. The vector according to claim 24, wherein said one or more co-expression elements are a) bidirectional promoters or are b) promoters, wherein the vector comprises a separate promoter for each of the nucleic acid sequences encoding the first polypeptide and the one or more immunostimulatory compounds.

26. The vector according to any of the previous claims, wherein the antigenic unit comprises one or more neoantigens or parts thereof, such as neoepitopes.

27. The vector according to claim 26, wherein the antigenic unit comprises several neoepitopes, such as several neoepitopes which are separated from each other by linkers.

28. The vector according to any of claims 26 to 27, wherein the antigenic unit further comprises one or more patient-present shared cancer antigens or parts thereof, such as patient-present shared cancer epitopes.

29. The vector according to any of claims 1 to 25, wherein the antigenic unit comprises one or more patient-present shared cancer antigens or parts thereof, such as patient-present shared cancer epitopes.

30. The vector according to any of claims 1 to 25, wherein the antigenic unit comprises one or more shared cancer antigens or parts thereof, such as shared cancer epitopes.

31. The vector according to any of claims 1 to 25, wherein the antigenic unit comprises one or more antigens derived from one or more pathogens or parts of such antigens.

32. The vector according to claim 31, wherein the antigenic unit comprises one or more full-length antigens derived from one or more pathogens or one or more parts of such full-lengths antigens or one or more full-lengths antigens derived from one or more pathogens and one or more parts of such full-lengths antigens.

33. The vector according to any of claims 31 to 32, wherein the antigenic unit comprises one or more parts of one or more antigens derived from one or more pathogens.

34. The vector according to claim 33, wherein such parts are B cell epitopes, such that the antigenic unit comprises one or more B cell epitopes derived from one or more pathogens.

35. The vector according to claim 33, wherein such parts are T cell epitopes, such that the antigenic unit comprises one or more T cell epitopes derived from one or more pathogens.

36. The vector according to any of claims 31 to 32, wherein the antigenic unit comprises (i) one or more full-length antigens derived from one or more pathogens or one or more parts of such antigens and (ii) one or more T cell epitopes derived from one or more pathogens.

37. The vector according to any of claims 31 to 36, wherein the one or more pathogens are selected from the group consisting of viruses, bacteria, fungi and parasites.

38. The vector according to any of the previous claims, wherein the targeting unit is or comprises a moiety that interacts with surface molecules on the antigen-presenting cells.

39. The vector according to claim 38, wherein the surface molecule is selected from the group consisting of MHC, HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5, preferably wherein the surface molecule is selected from the group consisting of HLA, hCD14, hCD40, hCLEC9A, human chemokine receptors, such as hCCR1, hCCR3, hCCR4, hCCR5, hCCR6, hCCR7, hCCR8 or hXCR1 and human Toll-like receptors such as hTLR-2, hTLR-4 or hTLR-5.

40. The vector according to any of claims 38 and 39, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II, anti-CD40, anti-TLR-2, anti-TLR-4, anti-TLR-5 or anti-CLEC9A, preferably wherein the targeting unit comprises or consists of soluble hCD40 ligand, hCCL4 and its isoforms, hCCL5, hCCL19, hCCL20, hCCL21, human macrophage inflammatory protein alpha including its isoforms, such human CCL3, human CCL3L1, human CCL3L2 and human CCL3L3, hXCL1, hXCL2, anti-HLA-DP, anti-HLA-DR, anti-pan HLA class II, anti-hCD40, anti-hTLR-2, anti-hTLR-4, anti-hTLR-5 or anti-hCLEC9.

41. The vector according to claim 40, wherein the targeting unit comprises or consists of human MIP-1α (LD78B, CCL3L1).

42. The vector according to any of the previous claims, wherein the multimerization unit is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4.

43. The vector according to claim 42, wherein the vector comprises a hinge region which has the ability to form one or more covalent bonds and is preferably Ig derived.

44. The vector according to any of claims 42 to 43, wherein the multimerization unit is a dimerization unit and said dimerization unit further comprises another domain that facilitates dimerization, preferably wherein the other domain is an immunoglobulin domain, more preferably an immunoglobulin constant domain.

45. The vector according to claim 44, wherein the other domain is a carboxyterminal C domain derived from IgG, preferably from IgG3.

46. The vector according to any of claims 44 to 45, wherein the dimerization unit further comprises a dimerization unit linker, such as glycine-serine rich linker, such as GGGSSGGGSG (SEQ ID NO: 134) and preferably wherein the dimerization unit linker connects the hinge region and the other domain that facilitates dimerization.

47. The vector according to any of claims 44 to 46, wherein the dimerization unit comprises hinge exon h1 and hinge exon h4, a dimerization unit linker and a CH3 domain of human IgG3.

48. The vector according to any of the previous claims, wherein the first nucleic acid sequence encodes a first polypeptide which further comprises a unit liker that connects the antigenic unit to the multimerization unit, and wherein the unit linker is a non-immunogenic linker and/or flexible or rigid linker.

49. The vector according to any of the previous claims, wherein the first nucleic acid sequence encodes a first polypeptide which further comprises a signal peptide and preferably wherein also the one or more further nucleic acid sequences further encode a signal peptide.

50. The vector according to any of the previous claims, wherein the vector is a viral vector, such as an RNA viral vector or DNA viral vector or a plasmid, such as an RNA plasmid or DNA plasmid.

51. A method of producing a vector as defined in any of the previous claims, the method comprising:
a) transfecting cells in vitro with the vector;
b) culturing said cells;
c) optionally, lysing the cells to release the vector from the cells; and
d) collecting and optionally purifying the vector.

52. A host cell comprising a vector as defined in any of claims 1 to 50, such as a host cell selected from the group consisting of prokaryote cells, yeast cells, insect cells, higher eukaryotic cells such as cells from animals or humans.

53. A vector as defined in any of claims 1 to 50 for use as a medicament

54. A pharmaceutical composition comprising the vector as defined in any of claims 1 to 50 and a pharmaceutically acceptable carrier or diluent.

55. The pharmaceutical composition according to claim 54, wherein the composition further comprises a transfection agent.

56. The pharmaceutical composition according to any of claims 54 to 55, wherein the composition comprises said vector, e.g. said DNA plasmid, in a range of from 0.1 to 10 mg.

57. A method of treating a subject having a disease or being in need of prevention of said disease, the method comprising administering to the subject a vector as defined in any of claims 1 to 50 or a pharmaceutical composition as defined in any of claims 54 to 56.

58. The method according to claim 57, wherein the vector or the pharmaceutical composition is administered in a therapeutically or prophylactically effective amount, such as administered by intradermal, intramuscular, or subcutaneous injection, or by mucosal or epithelial application, such as intranasal or oral.

59. A method of treating a subject having cancer, the method comprising administering to the subject a vector as defined in any of claims 1 to 30 and 38 to 50 or a pharmaceutical composition as defined in any of claims 54 to 56 comprising such vector.

60. The method according to claim 59, wherein the vector or the pharmaceutical composition is administered in a therapeutically effective amount, such as administered by intradermal, intramuscular, or subcutaneous injection, or by mucosal or epithelial application, such as intranasal or oral.

61. A method for treating a subject having an infectious disease or being in need of prevention of an infectious disease, the method comprising administering to the subject a vector as defined in any of claims 1 to 25 and 31 to 50 or a pharmaceutical composition as defined in any of claims 54 to 56, comprising such vector.

62. The method according to claim 61, wherein the vector or the pharmaceutical composition is administered in a therapeutically or prophylactically effective amount, such as administered by intradermal, intramuscular, or subcutaneous injection, or by mucosal or epithelial application, such as intranasal or oral.
